(19) United States
(12) Patent Application Publication
(10) Pub. No.: US 2025/0250232 A1
(43) Pub. Date: Aug. 7, 2025

(54) AZETIDINE DERIVATIVES AND USE THEREOF AS DIPEPTIDYL PEPTIDASE 1 INHIBITORS

(71) Applicant: CHIESI FARMACEUTICI S.P.A., Parma (IT)

(72) Inventors: Elisabetta ARMANI, Parma (IT); Sara GUARIENTO, Parma (IT); Andrea RIZZI, Parma (IT); Huw David VATER, Parma (IT); Hervè Pierre Clotaire VAN DE POËL, Parma (IT)

(73) Assignee: CHIESI FARMACEUTICI S.P.A., Parma (IT)

(21) Appl. No.: 18/880,877

(22) PCT Filed: Jul. 4, 2023
(86) PCT No.: PCT/EP2023/068311

§ 371 Date: Jan. 3, 2025

(30) Foreign Application Priority Data
Jul. 6, 2022 (EP) 22183409.6
Nov. 10, 2022 (EP) 22206777.9

(51) Int. Cl. C07D 205/04; A61K 31/397; A61K 31/403; A61K 31/422; A61K 31/423; A61K 31/438; A61K 31/4427; C07D 401/12; C07D 403/12; C07D 405/04; C07D 405/06; C07D 413/12; C07D 491/107

(52) U.S. Cl. CPC C07D 205/04; A61K 31/397; A61K 31/403; A61K 31/422; A61K 31/423; A61K 31/438; A61K 31/4427; C07D 401/12; C07D 403/12; C07D 405/04; C07D 405/06; C07D 413/12; C07D 491/107

(57) ABSTRACT

The present invention generally relates to compounds inhibiting dipeptidyl peptidase 1 activity; particularly the invention relates to compounds that are azetidine derivatives, including pharmaceutically acceptable salts thereof, methods of preparing such compounds, and therapeutic use thereof. The compounds of the invention may be useful for instance in the treatment of many disorders associated with DPP1 receptors mechanisms, in particular respiratory diseases.

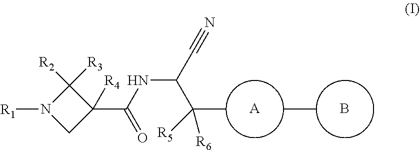

### FIELD OF THE INVENTION

The present invention generally relates to compounds inhibiting dipeptidyl peptidase 1 activity (hereinafter DPP1 inhibitors); the invention relates to compounds that are azetidine derivatives, including pharmaceutically acceptable salts thereof, methods of preparing such compounds, and therapeutic use thereof.

The compounds of the invention may be useful for instance in the treatment of many disorders associated with DPP1 receptors mechanisms.

### BACKGROUND OF THE INVENTION

Cathepsin C (Cat C) or dipeptidyl peptidase 1 (DPP1) is an amino dipeptidyl peptidase that is unique amongst the 11 human lysosomal cysteine cathepsins because of its oligomeric structure and its requirement for a halide ion (see e.g. Turk, D et al, EMBO J., 2001, 20, 6570-6582).

DPP1 plays a key role in the activation of the proinflammatory neutrophil serine proteases (NSPs), neutrophil elastase (NE), Proteinase 3 (Pr3), and Cathepsin G (CatG). Inhibition of DPP1 has therefore been implicated as a therapeutic treatment of diseases that carry a high neutrophilic burden: DPP1 activates NSPs by cleaving the N-terminal dipeptide during neutrophil maturation in the bone marrow. Inhibition of DPP1 in the bone marrow would therefore lead to neutrophils without stored active NE, Pr3, or CatG and has the potential to reduce the high local release of active NSPs that cause inflammation and neutrophil-driven lung damage (see e.g. Daniel Guay et al, Current Topics in Medicinal Chemistry, 2010, 10, 708-716; Korkmaz, B et al, Pharmacol. Rev. 2010, 62, 726-759).

The fact that Neutrophil elastase, Cathepsin G and Proteinase 3 seem to play significant roles in immunological and inflammatory diseases points to DPPI being a valid therapeutic target due to its central role in activating these proteases (see e.g. Adkison et al. 2002, J Clin Jnvest, 109, 363-271; Pham et al. 2004, J Immunol, 173, 7277-7281).

Bronchiectasis, along with chronic obstructive pulmonary disease (COPD), acute lung injury, acute respiratory distress syndrome, and cystic fibrosis (CF), are all conditions of severe pulmonary dysfunction resulting from a massive inflammatory response. The histological characteristic of these inflammatory lung diseases is the accumulation of neutrophils in the interstitium and alveoli of the lung. Neutrophil activation leads to the release of multiple cytotoxic products including reactive oxygen species and proteases (serine, cysteine, and metalloproteases). (see e.g. International Patent Application WO2018/022978, Astrazeneca.)

Inappropriate NE activity is implicated for example in the development of chronic obstructive pulmonary disease (COPD), and Cat C knockout mice are resistant to lung airspace enlargement and inflammatory cell infiltration in both cigarette smoke and ozone exposure models of COPD (see e.g. Turk, D et al, EMBO J., 2001, 20, 6570-6582).

A first generation of highly potent, selective and stable DPP1 covalent inhibitors led to the first oral clinical candidates. It is known from the International Patent Application WO2004/110988 (Combio) that certain nitrile derivatives are inhibitors of DPP1.

WO2010/128324 (Astrazeneca) relates to α-amino amide nitriles and their use as DPP1 inhibitors.

WO2015/110826 (Astrazeneca) relates to certain (2)-N-1(1)-1-cyano-2-phenylethyl]-1,4-oxazepane-2-carboxamide compounds that inhibit DPP1 activity. It was found that amide nitrile compound which bears β-amino acid possess potent DPP1 activity and/or have desirable pharmacological activity profiles (for example a decreased risk of binding to elastin rich tissue, such as the aorta).

Many DPP1 inhibitors in the state of art contain seven-membered cyclic β-amino acids: it was noted that as the ring size increased there was an improvement in enzyme and cellular potencies (see e.g. K Doyle, J. Med. Chem. 2016, 59, 9457-9472).

In this respect, the state of the art does not describe or suggest fourth-membered cyclic 3-amino acids derivatives, as azetidine of general formula (I), having a good inhibitor activity on receptors DPP1.

To date, there are still no DPP1 inhibitors on the market and therefore DPP1 inhibitors with high inhibitory activity, good PK properties and low toxicity remain an unmet clinical need.

### SUMMARY OF THE INVENTION

In a first aspect the invention refers to a compound of formula (I)

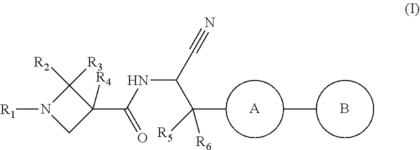

wherein
R₁ is H or selected from the group consisting of —(C₁-C₆)alkyl, —(C₁-C₆)haloalkyl, —(C₁-C₆)hydroxyalkyl, —(C₁-C₆)alkoxy, —(C₃-C₆)cycloalkyl, aryl, heterocycloalkyl or heteroaryl, wherein said —(C₁-C₆)alkyl is optionally substituted by one or more groups selected from —NR₉SO₂R₁₀, —SO₂NR₉R₁₀, —NR₉—C(O)R₁₀, —C(O)NR₉R₁₀, —NR₉R₁₀, aryl, —(C₃-C₆)cycloalkyl, heterocycloalkyl and heteroaryl;
R₂ and R₃ are independently H or selected from the group consisting of —(C₁-C₆)alkyl, halogen and —(C₁-C₆)haloalkyl;
R₄ is H or selected from the group consisting of halogen, —OR₇, —SR₈, —SO₂R₈, —(C₁-C₆)alkyl, —(C₁-C₆)haloalkyl, —(C₁-C₆)aminoalkyl, —(C₁-C₆)hydroxyalkyl, —(C₁-C₆)alkoxy, —(C₁-C₆)alkyl-S—(C₁-C₄)alkyl, heterocycloalkyl, aryl, heteroaryl, —(C₃-C₆)cycloalkyl, wherein any of such —(C₁-C₆)alkyl, —(C₁-C₆)haloalkyl, —(C₁-C₆)alkoxy, —(C₁-C₆)alkyl-S—(C₁-C₄)alkyl are optionally substituted by one or more groups selected from halogen, —NR₉SO₂R₁₀, —SO₂NR₉R₁₀, —C(O)NR₉R₁₀, —NR₉R₁₀, —NR₉—C(O)R₁₀, aryl, —(C₃-C₆)cycloalkyl, heterocycloalkyl and heteroaryl;
R₅ and R₆ are independently H or selected from the group consisting of —(C₁-C₆)alkyl, —(C₁-C₆)haloalkyl or fused together in a —(C₃-C₆)cycloalkyl, wherein said —(C₃-C₆)cycloalkyl is optionally substituted by one or more groups selected from halogen and —(C₁-C₆)alkyl;
R₇ and R₈ are independently H or selected from the group consisting of —(C₁-C₆)alkyl, —(C₁-C₆)haloalkyl, heterocycloalkyl, —(C₃-C₆)cycloalkyl, aryl, heteroaryl, wherein said —(C₁-C₆)alkyl is optionally substituted by one or more groups selected from aryl, —(C₃-C₆)cycloalkyl, heterocycloalkyl, —NR₉SO₂R₁₀, —SO₂NR₉R₁₀, —C(O)NR₉R₁₀, —NR₉—C(O)R₁₀, —NR₉R₁₀, and wherein any of such aryl, heteroaryl, —(C₃-C₆)cycloalkyl, heterocycloalkyl are optionally substituted by one or more halogen, —(C₁-C₆)alkyl, —(C₁-C₆)haloalkyl and —(C₁-C₆)hydroxyalkyl;
R₉ or R₁₀ are independently H or selected from the group consisting of —(C₁-C₆)haloalkyl, aryl, —(C₁-C₆)alkyl or fused together in an heterocycloalkyl or in —(C₃-C₆)cycloalkyl, wherein any of such heterocycloalkyl, —(C₃-C₆)cycloalkyl are optionally substituted by one or more —(C₁-C₆)alkyl;
A is monocyclic ring selected from aryl, heterocycloalkyl, —(C₃-C₆)cycloalkyl, wherein any of such aryl, heterocycloalkyl, —(C₃-C₆)cycloalkyl are optionally substituted by one or more halogen, oxo, —OR₇, —NR₉SO₂R₁₀, —SO₂NR₉R₁₀, —C(O)NR₉R₁₀, —NR₉—C(O)R₁₀, —NR₉R₁₀, —SR₈, —(C₁-C₆)alkyl, —(C₁-C₆)haloalkyl, —(C₁-C₆)hydroxyalkyl and —(C₁-C₆)amminoalkyl;
B is a ring selected from aryl, heteroaryl, each of said aryl or heteroaryl may be fused to a second saturated or unsaturated ring optionally containing one or more heteroatoms selected from N, S and O to form a bicyclic, tricyclic or a spiro tricyclic ring system, said B being optionally substituted with one or more substituent selected from halogen, —OR₇, —SR₈, oxo, cyano, —(C₁-C₆)haloalkyl, —(C₁-C₆)alkyl, —(C₁-C₆)amminoalkyl, aryl, heteroaryl, heterocycloalkyl, —(C₃-C₆)cycloalkyl, —NR₉R₁₀, —C(O)NR₉R₁₀, —NR₉—C(O)R₁₀, —SO₂NR₉R₁₀, —NR₉SO₂R₁₀, —(C₁-C₆)alkyl-C(O)NR₉R₁₀, —(C₁-C₆)alkyl-NR₉—C(O)R₁₀, —(C₁-C₆)alkyl-SO₂NR₉R₁₀, —(C₁-C₆)alkyl-NR₉SO₂R₁₀, —NH—C(O)—OR₇ and —O—C(O)—NR₉R₁₀;
and pharmaceutically acceptable salts or deuterated thereof.

In a second aspect, the invention refers to pharmaceutical composition comprising a compound of formula (I) in a mixture with one or more pharmaceutically acceptable carrier or excipient.

In a third aspect, the invention refers to a compound of formula (I) or a pharmaceutical composition for the use as a medicament.

In a further aspect, the invention refers to a compound of formula (I) or a pharmaceutical composition for use in treating disease, disorder, or condition associated with dysregulation of DDP1.

In a further aspect, the invention refers to a compound of formula (I) or a pharmaceutical composition for use in the prevention and/or treatment of an inflammatory or obstructive respiratory disease.

### DETAILED DESCRIPTION OF THE INVENTION

Unless otherwise provided, the term compound of formula (I) comprises in its meaning deuterated form, stereoisomer, tautomer or pharmaceutically acceptable salt or solvate.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ³C- or ¹⁴C-enriched carbon are within the scope of this invention.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable. Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium, or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or non-stoichiometric amount of the solvent molecules.

The term “heteroatom” refers to each atom different from C and H.

The term “stereoisomer” refers to isomers of identical constitution that differ in the arrangement of their atoms in space. Enantiomers and diastereomers are examples of stereoisomers.

The term “enantiomer” refers to one of a pair of molecular species that are mirror images of each other and are not superimposable.

The term “diastereomer” refers to stereoisomers that are not mirror images.

The term “racemate” or “racemic mixture” refers to a composition composed of equimolar quantities of two enantiomeric species, wherein the composition is devoid of optical activity.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996)).

The term “tautomer” refers to each of two or more isomers of a compound that exist together in equilibrium and are readily interchanged by migration of an atom or group within the molecule.

The term “IC₅₀” refers to the half maximal inhibitory concentration as a measure of the potency of a substance in inhibiting a specific biological or biochemical function.

The term “pIC₅₀” refers to the negative logarithm of the IC₅₀ value expressed as molar concentration.

The term “halogen” or “halogen atoms” or “halo” as used herein includes fluorine, chlorine, bromine, and iodine atom.

The term “cyano” refers to CN group.

The term “(Cₓ-C_{y})alkyl” wherein x and y are integers, refers to a straight or branched chain alkyl group having from x to y carbon atoms. The same term also includes the case in which one or more hydrogen atoms linked to the straight or branched chain alkyl group is replaced by one or more deuterium atoms. Thus, when x is 1 and y is 6, for example, the term includes methyl, ethyl, n-propyl, isopropyl, n-butyl, isobutyl, sec-butyl, t-butyl, n-pentyl and n-hexyl.

The term “(Cₓ-C_{y})haloalkyl” wherein x and y are integers, refers to the above defined “(Cₓ-C_{y})alkyl” groups wherein one or more hydrogen atoms are replaced by one or more halogen atoms, which can be the same or different. Examples of said “(Cₓ-C_{y}) haloalkyl” groups may thus include halogenated, poly-halogenated and fully halogenated alkyl groups wherein all hydrogen atoms are replaced by halogen atoms, e.g. trifluoromethyl or difluoromethyl.

The term “(Cₓ-C_{y})aminoalkyl” wherein x and y are integers, refer to the above defined “(Cₓ-C_{y})alkyl” groups wherein one or more hydrogen atoms are replaced by one or more amino group respectively. Examples of suitable “(Cₓ-C_{y})aminoalkyl” systems include, for instance, amminomethyl.

The term “(Cₓ-C_{y})cycloalkyl” wherein x and y are integers, refers to saturated cyclic hydrocarbon groups containing the indicated number of ring carbon atoms. Examples include cyclopropyl, cyclobutyl, cyclopentyl, cyclohexyl, cycloheptyl.

The term “(Cₓ-C_{y})hydroxyalkyl” wherein x and y are integers, refers to the above defined “(Cₓ-C_{y})alkyl” groups wherein one or more hydrogen atoms are replaced by one or more hydroxy (OH) respectively. Examples of suitable “(Cₓ-C_{y})hydroxyalkyl” systems include, for instance, hydroxymethyl.

The term “(Cₓ-C_{y})alkoxy” refers to a straight or branched hydrocarbon of the indicated number of carbons, attached through an oxygen bridge. Examples of suitable “(Cₓ-C_{y})alkoxy” systems include, for instance, methoxymethyl, ethoxymethyl, methoxyethyl.

The term “deuterium” refers to the isotopic deuterium of hydrogen (H).

The term “deuterated” refers to the case where the hydrogen atoms on an alkyl, cycloalkyl, aryl, heteroaryl group are substituted by at least one isotopic deuterium, with the upper limit of the number of deuterium substituents being equal to the sum of the number of hydrogen atoms that can be substituted. Unless otherwise indicated, the number of deuterium substituents is any integer between 1 and said upper limit, preferably substitution by 1 to 20 deuterium atoms, more preferably 1 to 10 deuterium atoms, more preferably 1 to 6 deuterium atoms, and further preferably 1 to 3 deuterium atoms.

The term “aryl” refers to mono cyclic carbon ring systems which have 6 ring atoms wherein the ring is aromatic. Examples of suitable aryl system include, for instance, phenyl.

The term “heteroaryl” refers to a mono- or bi-cyclic aromatic group containing 1 to 3 heteroatoms selected from S, N, and O, and includes groups having two rings, or one such monocyclic ring and one monocyclic aryl ring, which are fused through a common bond. Examples of suitable heteroaryl system include, for instance, isobenzofurane, dihydro-benzoxazole or indoline group.

The term “heterocycloalkyl” refers to a monocyclic satured or unsatured ring containing 1 to 3 heteroatoms selected from N, S and O. Examples of suitable “heterocycloalkyl” systems include, for instance, pyridine, pyrimidine, pyrazine, pyridazine, thiophene, pyrazole, imidazole, 4-morpholine, 1-piperazine, triazole, tetrazole, oxetane or oxazole group.

A bond pointing to a wavy or squiggly line, such as

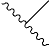

as used in structural formulas herein, depicts the bond that is the point of attachment of the moiety or substituent to the core or backbone structure.

A dash (“-”) that is not between two letters or symbols is meant to represent the point of attachment for a substituent.

The carbonyl group is herein preferably represented as —C(O)— as an alternative to the other common representations such as —CO—, —(CO)— or —C(═O)—.

Whenever basic amino or quaternary ammonium groups are present in the compounds of formula (I), physiologically acceptable anions may be present, selected among chloride, bromide, iodide, trifluoroacetate, formate, sulfate, phosphate, methanesulfonate, nitrate, maleate, acetate, citrate, fumarate, tartrate, oxalate, succinate, benzoate, p-toluenesulfonate, pamoate and naphthalene disulfonate. Likewise, in the presence of acidic groups such as COOH groups, corresponding physiological cation salts may be present as well, for instance including alkaline or alkaline earth metal ions.

As above indicated, the present invention refers to a series of compounds represented by the general formula (I) as herein below described in details, which are endowed with an inhibitor property versus receptor DPP1.

Differently from similar compounds of the prior art, the compounds of formula (I) are characterized by a fourth-membered cyclic β-amino acid moiety, as azetidine, surprisingly showing a good inhibitor activity on receptors DPP1.

In this respect, the state of the art does not describe or suggest fourth-membered cyclic 3-amino acids derivatives, as azetidine of general formula (I), having a good inhibitor activity on receptors DPP1.

In the state of the art a series of alternative 3-amino acids where the amino functionality is incorporated into the ring system, and in particular a series of six- and seven-membered ring analogues, were prepared and tested in human recombinant DPP1 isolated enzyme assay and DPP1 cell assay using U937 cell line. It was noted that as the ring size increased, from 5 to either 6 or 7, there was an improvement in their enzyme and cellular potencies, probably in part due to their increased lipophilicity (see e.g. K Doyle, J. Med. Chem. 2016, 59, 9457-9472).

In compounds of general formula (I) of the present invention, the presence of a 4-member ring maintains and improves a good in vitro profile in DPP1 enzyme assay, in human and rat. The compounds of formula (I) of the present invention are able to act as inhibitors of DPP1 in a substantive and effective way, particularly appreciated by the skilled person when looking at a suitable and efficacious compounds useful for treatment of inflammatory or obstructive respiratory disease, such as bronchiectasis.

As indicated in the experimental part, the compounds of formula (I) of the invention have an in vitro activity as shown in Table 15 wherein for each compound is reported the potency expressed as negative logarithmic value of half maximal inhibitory concentration in molar concentration (pIC50) on receptors DPP1.

As it can be appreciated, all the compounds of the present invention according to Table 15 show a potency with respect to their inhibitory activity on receptor DPP1, expressed as pIC50 values, higher than the value 5, preferably between the value 5 and 6 (+), preferably higher than the value 6, preferably between the value 6 and 7 (++), preferably between the value 7 and 8 (+++), more preferably higher than the value 8 (++++).

Thus, in one aspect the present invention relates to a compound of general formula (I) as DPP1 inhibitors.

In a first aspect the invention refers to a compound of formula (I)

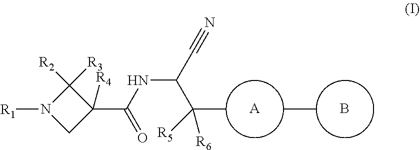

wherein
R₁ is H or selected from the group consisting of —(C₁-C₆)alkyl, —(C₁-C₆)haloalkyl, —(C₁-C₆)hydroxyalkyl, —(C₁-C₆)alkoxy, —(C₃-C₆)cycloalkyl, aryl, heterocycloalkyl or heteroaryl, wherein said —(C₁-C₆)alkyl is optionally substituted by one or more groups selected from —NR₉SO₂R₁₀, —SO₂NR₉R₁₀, —NR₉—C(O)R₁₀, —C(O)NR₉R₁₀, —NR₉R₁₀, aryl, —(C₃-C₆)cycloalkyl, heterocycloalkyl and heteroaryl;
R₂ and R₃ are independently H or selected from the group consisting of —(C₁-C₆)alkyl, halogen and —(C₁-C₆)haloalkyl;
R₄ is H or selected from the group consisting of halogen, —OR₇, —SR₈, —SO₂R₈, —(C₁-C₆)alkyl, —(C₁-C₆)haloalkyl, —(C₁-C₆)aminoalkyl, —(C₁-C₆)hydroxyalkyl, —(C₁-C₆)alkoxy, —(C₁-C₆)alkyl-S—(C₁-C₄)alkyl, heterocycloalkyl, aryl, heteroaryl, —(C₃-C₆)cycloalkyl, wherein any of such —(C₁-C₆)alkyl, —(C₁-C₆)haloalkyl, —(C₁-C₆)alkoxy, —(C₁-C₆)alkyl-S—(C₁-C₄)alkyl may be optionally substituted by one or more groups selected from halogen, —NR₉SO₂R₁₀, —SO₂NR₉R₁₀, —C(O)NR₉R₁₀, —NR₉R₁₀, —NR₉—C(O)R₁₀, aryl, —(C₃-C₆)cycloalkyl, heterocycloalkyl and heteroaryl;
R₅ and R₆ are independently H or selected from the group consisting of —(C₁-C₆)alkyl, —(C₁-C₆)haloalkyl or fused together in a —(C₃-C₆)cycloalkyl, wherein said —(C₃-C₆)cycloalkyl may be optionally substituted by one or more groups selected from halogen and —(C₁-C₆)alkyl;
R₇ and R₈ are independently H or selected from the group consisting of —(C₁-C₆)alkyl, —(C₁-C₆)haloalkyl, heterocycloalkyl, —(C₃-C₆)cycloalkyl, aryl, heteroaryl, wherein said —(C₁-C₆)alkyl is optionally substituted by one or more groups selected from aryl, —(C₃-C₆)cycloalkyl, heterocycloalkyl, —NR₉SO₂R₁₀, —SO₂NR₉R₁₀, —C(O)NR₉R₁₀, —NR₉—C(O)R₁₀, —NR₉R₁₀, and wherein any of such aryl, heteroaryl, —(C₃-C₆)cycloalkyl, heterocycloalkyl are optionally substituted by one or more halogen, —(C₁-C₆)alkyl, —(C₁-C₆)haloalkyl and —(C₁-C₆)hydroxyalkyl;
R₉ or R₁₀ are independently H or selected from the group consisting of —(C₁-C₆)haloalkyl, aryl, —(C₁-C₆)alkyl or fused together in an heterocycloalkyl or in —(C₃-C₆)cycloalkyl, wherein any of such heterocycloalkyl, —(C₃-C₆)cycloalkyl are optionally substituted by one or more —(C₁-C₆)alkyl;
A is monocyclic ring selected from aryl, heterocycloalkyl, —(C₃-C₆)cycloalkyl, wherein any of such aryl, heterocycloalkyl, —(C₃-C₆)cycloalkyl are optionally substituted by one or more halogen, oxo, —OR₇, —NR₉SO₂R₁₀, —SO₂NR₉R₁₀, —C(O)NR₉R₁₀, —NR₉—C(O)R₁₀, —NR₉R₁₀, —SR₈, —(C₁-C₆)alkyl, —(C₁-C₆)haloalkyl, —(C₁-C₆)hydroxyalkyl and —(C₁-C₆)amminoalkyl;
B is a ring selected from aryl, heteroaryl, each of said aryl or heteroaryl may be fused to a second saturated or unsaturated ring optionally containing one or more heteroatoms selected from N, S and O to form a bicyclic, tricyclic or a spiro tricyclic ring system, said B being optionally substituted with one or more substituent selected from halogen, —OR₇, —SR₈, oxo, cyano, —(C₁-C₆)haloalkyl, —(C₁-C₆)alkyl, —(C₁-C₆)amminoalkyl, aryl, heteroaryl, heterocycloalkyl, —(C₃-C₆)cycloalkyl, —NR₉R₁₀, —C(O)NR₉R₁₀, —NR₉—C(O)R₁₀, —SO₂NR₉R₁₀, —NR₉SO₂R₁₀, —(C₁-C₆)alkyl-C(O)NR₉R₁₀, —(C₁-C₆)alkyl-NR₉—C(O)R₁₀, —(C₁-C₆)alkyl-SO₂NR₉R₁₀, —(C₁-C₆)alkyl-NR₉SO₂R₁₀, —NH—C(O)—OR₇ and —O—C(O)—NR₉R₁₀;
and pharmaceutically acceptable salts or deuterated thereof.

All the listed groups for each of the variable moieties R₁, R₂, R₃, R₄, R₅, R₆, R₇, R₈, R₉, R₁₀, A and B of the compounds of the invention have to be intended as alternatives and may be combined with each other in embodiments which are included in the scope of the invention.

In one embodiment R₁ is H or selected from the group consisting of —(C₁-C₆)alkyl, —(C₁-C₆)haloalkyl, —(C₁-C₆)hydroxyalkyl, —(C₁-C₆)alkoxy, —(C₃-C₆)cycloalkyl, aryl, heterocycloalkyl or heteroaryl, wherein said —(C₁-C₆)alkyl is optionally substituted by one or more groups selected from —NR₉SO₂R₁₀, —SO₂NR₉R₁₀, —NR₉—C(O)R₁₀, —C(O)NR₉R₁₀, —NR₉R₁₀, aryl, —(C₃-C₆)cycloalkyl, heterocycloalkyl and heteroaryl.

In another embodiment R₁ is H or selected from the group consisting of —(C₁-C₆)alkyl or heterocycloalkyl, wherein said —(C₁-C₆)alkyl may be optionally substituted by one or more groups selected from —NR₉SO₂R₁₀, —SO₂NR₉R₁₀, —NR₉—C(O)R₁₀, —C(O)NR₉R₁₀, —NR₉R₁₀, aryl, —(C₃-C₆)cycloalkyl, heterocycloalkyl and heteroaryl. In a preferred embodiment R₁ is H or selected from the group consisting of —(C₁-C₄)alkyl or heterocycloalkyl, wherein said —(C₁-C₄)alkyl may be optionally substituted by one or more groups selected from —NR₉SO₂R₁₀, —SO₂NR₉R₁₀, —NR₉—C(O)R₁₀, —C(O)NR₉R₁₀, —NR₉R₁₀, aryl, —(C₃-C₆)cycloalkyl, heterocycloalkyl and heteroaryl. In a more preferred embodiment R₁ is H or a group selected from methyl, ethyl or isopropyl. In another preferred embodiment R₁ is heterocycloalkyl.

In one embodiment R₂ and R₃ are independently H or selected from the group consisting of —(C₁-C₆)alkyl, halogen and —(C₁-C₆)haloalkyl. In a preferred embodiment R₂ and R₃ are independently H or —(C₁-C₆)alkyl. In a preferred embodiment R₂ and R₃ are independently H or —(C₁-C₄)alkyl. In a more preferred embodiment R₂ and R₃ are independently H or Me or Et. In a more preferred embodiment R₂ and R₃ are independently Me. In another preferred embodiment R₂ and R₃ are H.

In one embodiment R₄ is H or selected from the group consisting of halogen, —OR₇, —SR₈, —SO₂R₈, —(C₁-C₆)alkyl, —(C₁-C₆)haloalkyl, —(C₁-C₆)aminoalkyl, —(C₁-C₆)hydroxyalkyl, —(C₁-C₆)alkoxy, —(C₁-C₆)alkyl-S—(C₁-C₄)alkyl, heterocycloalkyl, aryl, heteroaryl, —(C₃-C₆)cycloalkyl, wherein any of such —(C₁-C₆)alkyl, —(C₁-C₆)haloalkyl, —(C₁-C₆)alkoxy, —(C₁-C₆)alkyl-S—(C₁-C₄)alkyl may be optionally substituted by one or more groups selected from halogen, —NR₉SO₂R₁₀, —SO₂NR₉R₁₀, —C(O)NR₉R₁₀, —NR₉R₁₀, —NR₉—C(O)R₁₀, aryl, —(C₃-C₆)cycloalkyl, heterocycloalkyl and heteroaryl.

In a preferred embodiment R₄ is H or selected from the group consisting of halogen, —OR₇, —SR₈, —SO₂R₈, —(C₁-C₆)alkyl, —(C₁-C₆)haloalkyl, —(C₁-C₆)alkoxy, heterocycloalkyl, aryl, heteroaryl, —(C₃-C₆)cycloalkyl, wherein any of such —(C₁-C₆)alkyl, —(C₁-C₆)haloalkyl, —(C₁-C₆)alkoxy may be optionally substituted by one or more groups selected from halogen, —NR₉SO₂R₁₀, —SO₂NR₉R₁₀, —C(O)NR₉R₁₀, —NR₉R₁₀, —NR₉—C(O)R₁₀, aryl, —(C₃-C₆)cycloalkyl, heterocycloalkyl and heteroaryl.

In a more preferred embodiment R₄ is selected from the group consisting of halogen, —OR₇, —SR₈, —SO₂R₈, —(C₁-C₆)alkyl, —(C₁-C₆)haloalkyl, —(C₁-C₆)aminoalkyl, —(C₁-C₆)hydroxyalkyl, —(C₁-C₆)alkoxy, —(C₁-C₆)alkyl-S—(C₁-C₄)alkyl, heterocycloalkyl, aryl, heteroaryl, —(C₃-C₆)cycloalkyl, wherein any of such —(C₁-C₆)alkyl, —(C₁-C₆)haloalkyl, —(C₁-C₆)alkoxy, —(C₁-C₆)alkyl-S—(C₁-C₄)alkyl may be optionally substituted by one or more groups selected from halogen, —NR₉SO₂R₁₀, —SO₂NR₉R₁₀, —C(O)NR₉R₁₀, —NR₉R₁₀, —NR₉—C(O)R₁₀, aryl, —(C₃-C₆)cycloalkyl, heterocycloalkyl and heteroaryl.

In one embodiment R₄ is H. In another embodiment R₄ is halogen, selected from fluorine, chlorine, bromine, and iodine atom. In preferred embodiment R₄ is fluorine.

In another embodiment R₄ is —OR₇, wherein R₇ is H or selected from the group consisting of —(C₁-C₆)alkyl, —(C₁-C₆)haloalkyl, aryl and —(C₁-C₆)heterocycloalkyl. In a preferred embodiment, R₄ is —OR₇, wherein R₇ is H. In another preferred embodiment, R₄ is —OR₇, wherein R₇ is Me, Et, ⁱPr. In another preferred embodiment, R₄ is —OR₇, wherein R₇ is aryl.

In another embodiment R₄ is —(C₁-C₆)alkyl. In a preferred embodiment R₄ is —(C₁-C₄)alkyl. In a more preferred embodiment, R₄ is selected from Me or Et.

In another embodiment R₄ is —(C₁-C₆)haloalkyl, wherein halogen can be selected from fluorine, chlorine, bromine, and iodine atom. In a preferred embodiment, R₄ is —(C₁-C₄)haloalkyl, wherein halogen is fluorine. In a preferred embodiment, R₄ is fluoromethyl, trifluoromethyl or difluoromethyl. In a more preferred embodiment, R₄ is difluoromethyl. In another preferred embodiment, R₄ is trifluoromethyl.

In another embodiment, R₄ is selected from the group consisting of —SR₈ and —SO₂R₈.

In another embodiment, R₄ is —(C₁-C₆)alkoxy. In a preferred embodiment, R₄ is —(C₁-C₄)alkoxy. In a more preferred embodiment, R₄ is methoxymethyl. In another preferred embodiment, R₄ is ethoxymethyl. In another preferred embodiment, R₄ is methoxyethyl.

In one embodiment, R₅ and R₆ are independently H or selected from the group consisting of —(C₁-C₆)alkyl, —(C₁-C₆)haloalkyl or fused together in a —(C₃-C₆)cycloalkyl, wherein said —(C₃-C₆)cycloalkyl may be optionally substituted by one or more groups selected from halogen and —(C₁-C₆)alkyl. In a preferred embodiment, R₅ and R₆ are independently H or —(C₁-C₆)alkyl. In a preferred embodiment, R₅ and Rare H or —(C₁-C₄)alkyl. In another preferred embodiment, R₅ and R₆ are fused together in a —(C₃-C₆)cycloalkyl. In a more preferred embodiment, R₅ and R₆ are fused together in a cyclopropyl.

In one embodiment R₇ and R₈ are independently H or selected from the group consisting of —(C₁-C₆)alkyl, —(C₁-C₆)haloalkyl, heterocycloalkyl, —(C₃-C₆)cycloalkyl, aryl, heteroaryl, wherein said —(C₁-C₆)alkyl may be optionally substituted by one or more groups selected from aryl, —(C₃-C₆)cycloalkyl, heterocycloalkyl, —NR₉SO₂R₁₀, —SO₂NR₉R₁₀, —C(O)NR₉R₁₀, —NR₉—C(O)R₁₀, —NR₉R₁₀, and wherein any of such aryl, heteroaryl, —(C₃-C₆)cycloalkyl, heterocycloalkyl may be optionally substituted by one or more halogen, —(C₁-C₆)alkyl, —(C₁-C₆)haloalkyl and —(C₁-C₆)hydroxyalkyl.

In another embodiment R₇ and R₈ are independently —(C₁-C₆)alkyl, wherein said —(C₁-C₆)alkyl may be optionally substituted by one or more groups selected from aryl, —(C₃-C₆)cycloalkyl, heterocycloalkyl, —NR₉SO₂R₁₀, —SO₂NR₉R₁₀, —C(O)NR₉R₁₀, —NR₉—C(O)R₁₀, —NR₉R₁₀. In a preferred embodiment R₇ and R₈ are independently H or optionally substituted —(C₁-C₄)alkyl; in another preferred embodiment R₇ and R₈ are H or Me.

In another embodiment R₇ and R₈ are independently H or heterocycloalkyl, wherein said heterocycloalkyl may be optionally substituted by one or more halogen, —(C₁-C₆)alkyl, —(C₁-C₆)haloalkyl, —(C₁-C₆)hydroxyalkyl.

In another embodiment R₇ and R₈ are independently H or aryl, wherein said aryl may be optionally substituted by one or more halogen, —(C₁-C₆)alkyl, —(C₁-C₆)haloalkyl, —(C₁-C₆)hydroxyalkyl.

In another embodiment R₇ and R₈ are independently H or heteroaryl, wherein said heteroaryl may be optionally substituted by one or more halogen, —(C₁-C₆)alkyl, —(C₁-C₆)haloalkyl, —(C₁-C₆)hydroxyalkyl.

In another embodiment R₇ and R₈ are independently H or —(C₃-C₆)cycloalkyl, wherein said —(C₃-C₆)cycloalkyl may be optionally substituted by one or more halogen, —(C₁-C₆)alkyl, —(C₁-C₆)haloalkyl, —(C₁-C₆)hydroxyalkyl.

In one embodiment R₉ or R₁₀ are independently H or selected from the group consisting of —(C₁-C₆)haloalkyl, aryl, —(C₁-C₆)alkyl or fused together in an heterocycloalkyl or in —(C₃-C₆)cycloalkyl, wherein any of such heterocycloalkyl, —(C₃-C₆)cycloalkyl may be optionally substituted by one or more —(C₁-C₆)alkyl.

In one embodiment R₉ or R₁₀ are independently H or —(C₁-C₆)alkyl. In a preferred embodiment R₉ or R₁₀ are independently H or —(C₁-C₄)alkyl. In a more preferred embodiment R₉ or R₁₀ are H.

In another embodiment R₉ or R₁₀ are fused together in —(C₃-C₆)cycloalkyl wherein —(C₃-C₆)cycloalkyl may be optionally substituted by one or more —(C₁-C₆)alkyl.

In another embodiment R₉ or R₁₀ are fused together in heterocycloalkyl, wherein heterocycloalkyl may be optionally substituted by one or more —(C₁-C₆)alkyl.

In one embodiment A is monocyclic ring selected from aryl, heterocycloalkyl, —(C₃-C₆)cycloalkyl, wherein any of such aryl, heterocycloalkyl, —(C₃-C₆)cycloalkyl may be optionally substituted by one or more halogen, oxo, —OR₇, —NR₉SO₂R₁₀, —SO₂NR₉R₁₀, —C(O)NR₉R₁₀, —NR₉—C(O)R₁₀, —NR₉R₁₀, —SR₈, —(C₁-C₆)alkyl, —(C₁-C₆)haloalkyl, —(C₁-C₆)hydroxyalkyl and —(C₁-C₆)amminoalkyl.

In another embodiment A is a monocyclic ring selected from aryl or heterocycloalkyl, wherein said aryl or heterocycloalkyl may be optionally substituted by one or more halogen, —(C₁-C₆)alkyl, —(C₁-C₆)haloalkyl, —(C₁-C₆)hydroxyalkyl and —(C₁-C₆)amminoalkyl.

In a preferred embodiment A is aryl, wherein said aryl may be optionally substituted by one or more halogen, —(C₁-C₆)alkyl, —(C₁-C₆)haloalkyl, —(C₁-C₆)hydroxyalkyl and —(C₁-C₆)amminoalkyl. In a more preferred embodiment A is aryl, wherein said aryl may be optionally substituted by one or more halogen, —(C₁-C₄)alkyl, —(C₁-C₄)haloalkyl, —(C₁-C₄)hydroxyalkyl and —(C₁—C₄)amminoalkyl. In another preferred embodiment, A is aryl optionally substituted by one or more halogen, selected from fluorine, chlorine, bromine, and iodine atom. In a more preferred embodiment A is aryl optionally substituted by one or more fluorine.

In another embodiment, A is a monocyclic heterocycloalkyl, wherein heterocycloalkyl may be optionally substituted by one or more halogen, —(C₁-C₆)alkyl, —(C₁-C₆)haloalkyl, —(C₁-C₆)hydroxyalkyl and —(C₁-C₆)amminoalkyl. In a preferred embodiment, A is heterocycloalkyl, wherein heterocycloalkyl may be optionally substituted by one or more halogen, —(C₁-C₄)alkyl, —(C₁-C₄)haloalkyl, —(C₁-C₄)hydroxyalkyl and —(C₁-C₄)amminoalkyl. In a preferred embodiment A is pyridine optionally substituted by one or more halogen.

In another embodiment, A is —(C₃-C₆)cycloalkyl, wherein —(C₃-C₆)cycloalkyl may be optionally substituted by one or more halogen, —(C₁-C₆)alkyl, —(C₁-C₆)haloalkyl, —(C₁-C₆)hydroxyalkyl and —(C₁-C₆)amminoalkyl. In a preferred embodiment A is —(C₃-C₆)cycloalkyl, wherein —(C₃-C₆)cycloalkyl may be optionally substituted by one or more halogen, —(C₁-C₄)alkyl, —(C₁-C₄)haloalkyl, —(C₁-C₄)hydroxyalkyl and —(C₁-C₄)amminoalkyl.

In one embodiment B is a ring selected from aryl, heteroaryl, each of said aryl or heteroaryl may be fused to a second saturated or unsaturated ring optionally containing one or more heteroatoms selected from N, S and O to form a bicyclic, tricyclic or a spiro tricyclic ring system, said B being optionally substituted with one or more substituent selected from halogen, —OR₇, —SR₈, oxo, cyano, —(C₁-C₆)haloalkyl, —(C₁-C₆)alkyl, —(C₁-C₆)amminoalkyl, aryl, heteroaryl, heterocycloalkyl, —(C₃-C₆)cycloalkyl, —NR₉R₁₀, —C(O)NR₉R₁₀, —NR₉—C(O)R₁₀, —SO₂NR₉R₁₀, —NR₉SO₂R₁₀, —(C₁-C₆)alkyl-C(O)NR₉R₁₀, —(C₁-C₆)alkyl-NR₉—C(O)R₁₀, —(C₁-C₆)alkyl-SO₂NR₉R₁₀, —(C₁-C₆)alkyl-NR₉SO₂R₁₀, —NH—C(O)—OR₇ and —O—C(O)—NR₉R₁₀.

In a preferred embodiment B is a ring selected from aryl, heteroaryl, said aryl, heteroaryl being optionally substituted with one or more substituent selected from halogen, —OR₇, —SR₈, oxo, cyano, —(C₁-C₆)haloalkyl, —(C₁-C₆)alkyl, —(C₁-C₆)amminoalkyl, aryl, heteroaryl, heterocycloalkyl, —(C₃-C₆)cycloalkyl, —NR₉R₁₀, —C(O)NR₉R₁₀, —NR₉—C(O)R₁₀, —SO₂NR₉R₁₀, —NR₉SO₂R₁₀, —(C₁-C₆)alkyl-C(O)NR₉R₁₀, —(C₁-C₆)alkyl-NR₉—C(O)R₁₀, —(C₁-C₆)alkyl-SO₂NR₉R₁₀, —(C₁-C₆)alkyl-NR₉SO₂R₁₀, —NH—C(O)—OR₇ and —O—C(O)—NR₉R₁₀. In a preferred embodiment B is a ring selected from aryl or heteroaryl, said aryl or heteroaryl being optionally substituted with one or more substituent selected from halogen, —OR₇, oxo, cyano, —(C₁-C₆)haloalkyl, —(C₁-C₆)alkyl, —(C₁-C₆)amminoalkyl, —NR₉R₁₀, —C(O)NR₉R₁₀, —NR₉—C(O)R₁₀, —SO₂NR₉R₁₀, —NR₉SO₂R₁₀. In a more preferred embodiment B is a ring selected from aryl or heteroaryl, said aryl or heteroaryl being optionally substituted with one or more substituent selected from halogen, oxo, cyano, —(C₁-C₆)haloalkyl, —(C₁-C₆)alkyl, —(C₁-C₆)amminoalkyl. In a more preferred embodiment B is a ring selected from aryl or heteroaryl, said aryl or heteroaryl being optionally substituted with one or more substituent selected from oxo, cyano, —(C₁-C₆)alkyl. In another preferred embodiment B is a ring selected from aryl or heteroaryl, said aryl or heteroaryl being optionally substituted with one or more substituent selected from oxo, cyano, —(C₁-C₄)alkyl.

In another preferred embodiment B is a ring selected from aryl, heteroaryl, said heteroaryl may be fused to a second saturated or unsaturated ring optionally containing one or more heteroatoms selected from N, S and O to form a tricyclic or a spiro tricyclic ring system, said B being optionally substituted with one or more substituent selected from halogen, —OR₇, —SR₈, oxo, cyano, —(C₁-C₆)haloalkyl, —(C₁-C₆)alkyl, —(C₁-C₆)amminoalkyl, aryl, heteroaryl, heterocycloalkyl, —(C₃-C₆)cycloalkyl, —NR₉R₁₀, —C(O)NR₉R₁₀, —NR₉—C(O)R₁₀, —SO₂NR₉R₁₀, —NR₉SO₂R₁₀, —(C₁-C₆)alkyl-C(O)NR₉R₁₀, —(C₁-C₆)alkyl-NR₉—C(O)R₁₀, —(C₁-C₆)alkyl-SO₂NR₉R₁₀, —(C₁-C₆)alkyl-NR₉SO₂R₁₀, —NH—C(O)—OR₇ and —O—C(O)—NR₉R₁₀.

In a preferred embodiment B is aryl, said aryl being optionally substituted with one or more substituent selected from halogen, —OR₇, —SR₈, oxo, cyano, —(C₁-C₆)haloalkyl, —(C₁-C₆)alkyl, —(C₁-C₆)amminoalkyl, aryl, heteroaryl, heterocycloalkyl, —(C₃-C₆)cycloalkyl, —NR₉R₁₀, —C(O)NR₉R₁₀, —NR₉—C(O)R₁₀, —SO₂NR₉R₁₀, —NR₉SO₂R₁₀, —(C₁-C₆)alkyl-C(O)NR₉R₁₀, —(C₁-C₆)alkyl-NR₉—C(O)R₁₀, —(C₁-C₆)alkyl-SO₂NR₉R₁₀, —(C₁-C₆)alkyl-NR₉SO₂R₁₀, —NH—C(O)—OR₇ and —O—C(O)—NR₉R₁₀. In a preferred embodiment, B is aryl, optionally substituted with one or more substituent selected from halogen, —OR₇, oxo, cyano, —(C₁-C₆)haloalkyl, —SO₂—R₉, wherein R₉ is H or heterocycloalkyl. In a more preferred embodiment, B is aryl, optionally substituted with one or more substituent selected from halogen, cyano, —OR₇, —SO₂—R₉, wherein R₉ is methyl-piperazine.

In a more preferred embodiment B is aryl, said aryl being optionally substituted with one or more substituent selected from halogen, selected from fluorine, chlorine, bromine, and iodine atom. In more preferred embodiment B is aryl, said aryl being optionally substituted with one or more fluorine. In another preferred embodiment B is aryl, said aryl being optionally substituted with one or more cyano. In another preferred embodiment B is aryl, said aryl being optionally substituted with one or more —OR₇. In another preferred embodiment B is aryl, said aryl being optionally substituted with one or more —(C₁-C₄)haloalkyl, —(C₁-C₄)alkyl, —(C₁-C₄)amminoalkyl.

In another preferred embodiment B is heteroaryl, said heteroaryl being optionally substituted with one or more substituent selected from halogen, —OR₇, —SR₈, oxo, cyano, —(C₁-C₆)haloalkyl, —(C₁-C₆)alkyl, —(C₁-C₆)amminoalkyl, aryl, heteroaryl, heterocycloalkyl, —(C₃-C₆)cycloalkyl, —NR₉R₁₀, —C(O)NR₉R₁₀, —NR₉—C(O)R₁₀, —SO₂NR₉R₁₀, —NR₉SO₂R₁₀, —(C₁-C₆)alkyl-C(O)NR₉R₁₀, —(C₁-C₆)alkyl-NR₉—C(O)R₁₀, —(C₁-C₆)alkyl-SO₂NR₉R₁₀, —(C₁-C₆)alkyl-NR₉SO₂R₁₀, —NH—C(O)—OR₇ and —O—C(O)—NR₉R₁₀.

In a more preferred embodiment B is heteroaryl selected from indoline, dihydro-benzoxazole, said heteroaryl being optionally substituted with one or more substituent selected from oxo and —(C₁-C₆)alkyl.

In another embodiment B is heteroaryl, said heteroaryl may be fused to second saturated or unsaturated ring optionally containing one or more heteroatoms selected from N and O to form a tricyclic or spiro tricyclic ring system, said heteroaryl being optionally substituted with one or more substituent selected from halogen, —OR₇, —SR₈, oxo, cyano, —(C₁-C₆)haloalkyl, —(C₁-C₆)alkyl, —(C₁-C₆)amminoalkyl, aryl, heteroaryl, heterocycloalkyl, —(C₃-C₆)cycloalkyl, —NR₉R₁₀, —C(O)NR₉R₁₀, —NR₉—C(O)R₁₀, —SO₂NR₉R₁₀, —NR₉SO₂R₁₀, —(C₁-C₆)alkyl-C(O)NR₉R₁₀, —(C₁-C₆)alkyl-NR₉—C(O)R₁₀, —(C₁-C₆)alkyl-SO₂NR₉R₁₀, —(C₁-C₆)alkyl-NR₉SO₂R₁₀, —NH—C(O)—OR₇ and —O—C(O)—NR₉R₁₀. In a preferred embodiment B is heteroaryl, said heteroaryl may be fused to second saturated or unsaturated ring optionally containing one or more heteroatoms selected from N and O to form a tricyclic or spiro tricyclic ring system, said heteroaryl being optionally substituted with one or more substituent selected from halogen, —OR₇, oxo, —(C₁-C₆)haloalkyl, —(C₁-C₆)alkyl, heterocycloalkyl and —SO₂—R₉. In a more preferred embodiment B is heteroaryl, said heteroaryl may be fused to second saturated or unsaturated ring optionally containing one or more heteroatoms selected from N and O to form a tricyclic or spiro tricyclic ring system, said heteroaryl being optionally substituted with one or more substituent selected from oxo, —(C₁-C₆)alkyl and heterocycloalkyl. In a more preferred embodiment B is heteroaryl, said heteroaryl may be fused to second saturated or unsaturated ring optionally containing one or more heteroatoms selected from N and O to form a tricyclic or spiro tricyclic ring system, said heteroaryl being optionally substituted with one or more substituent selected from —(C₁-C₆)alkyl and heterocycloalkyl.

In a more preferred embodiment B is a ring selected from optionally substituted aryl, heteroaryl, said heteroaryl is selected from the group consisting of indoline, dihydro-benzoxazole and 3H-isobenzofurane. In another preferred embodiment B is an heteroaryl selected from the group consisting of optionally substituted indoline, dihydro-benzoxazole and 3H-isobenzofurane.

In a preferred embodiment B is a ring selected from optionally substituted aryl, heteroaryl, said heteroaryl selected from the group consisting of (II), (III) and (IV). In a preferred embodiment B is heteroaryl, said heteroaryl selected from (II), (III) and (IV).

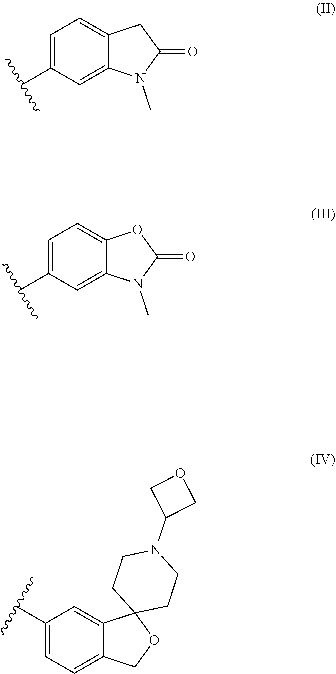 [SMILES: *c1ccc2c(c1)N(C)C(=O)C2 ; *c1ccc2oc(=O)n(C)c2c1 ; *c1ccc2c(c1)C1(CCN(C3COC3)CC1)OC2]

In a preferred embodiment, B is indoline, said indoline being optionally substituted with one or more substituent selected from oxo and —(C₁-C₆)alkyl. In a more preferred embodiment, B is indoline, said indoline being optionally substituted with one or more substituent selected from oxo and —(C₁-C₄)alkyl.

In another preferred embodiment B is dihydro-benzoxazole, said dihydro-benzooxazole being optionally substituted with one or more substituent selected from oxo and —(C₁-C₆)alkyl. In a more preferred embodiment B is dihydro-benzoxazole, said dihydro-benzoxazole being optionally substituted with one or more substituent selected from oxo and —(C₁-C₄)alkyl.

In another preferred embodiment B is 3H-isobenzofurane which may be fused to a second saturated or unsaturated ring optionally containing one or more heteroatoms selected from N, S and O to form a tricyclic or a spiro tricyclic ring system, wherein such ring may be optionally substituent with heterocycloalkyl.

In a more preferred embodiment B is 3H-spiro-isobenzofuran-1,4′-piperidine, optionally substituted with heterocycloalkyl. In more preferred embodiment B is 3H-spiro-isobenzofuran-1,4′-piperidine, optionally substituted with oxetane.

All the listed groups for each of the variable moieties R₁, R₂, R₃, R₄, R₅, R₆, R₇, R₈, R₉, R₁₀, A and B of the compounds of the invention have to be intended as alternatives and may be combined with each other in embodiments which are included in the scope of the invention.

In a preferred embodiment, the invention refers to a compound of formula (Ia)

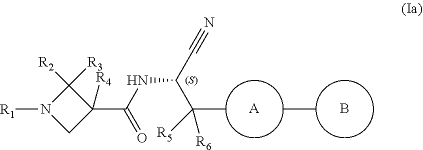

Wherein R₁, R₂, R₃, R₄, R₅, R₆, R₇, R₈, R₉, R₁₀, and cycle A and B are defined as above.

In a preferred embodiment, the invention refers to a compound of formula (I), wherein R₁ is H or selected from the group consisting of —(C₁-C₆)alkyl, heterocycloalkyl or heteroaryl, wherein said —(C₁-C₆)alkyl may be optionally substituted by one or more groups selected from aryl, —(C₃-C₆)cycloalkyl, —C(O)NR₉R₁₀, heterocycloalkyl and heteroaryl;
R₂ and R₃ are independently H or —(C₁-C₆)alkyl; and
R₄, R₅, R₆, R₇, R₈, R₉, R₁₀, and cycle A and B are defined as above.

In a preferred embodiment, the invention refers to a compound of formula (Ia), wherein R₁ is H, represented as formula (Ib)

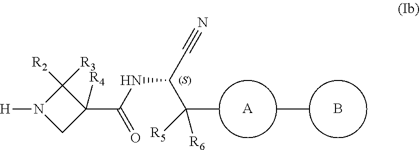

Wherein R₂, R₃, R₄, R₅, R₆, R₇, R₈, R₉, R₁₀, and cycle A and B are defined as above.

In another preferred embodiment, the invention refers to a compound of formula (Ia), wherein R₁ is methyl, represented as formula (Ic)

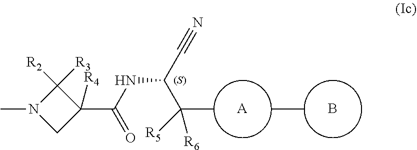

Wherein R₂, R₃, R₄, R₅, R₆, R₇, R₈, R₉, R₁₀, and cycle A and B are defined as above.

In another preferred embodiment, the invention refers to a compound of formula (I), wherein R₂ and R₃ are independently H or —(C₁-C₆)alkyl.

In a preferred embodiment, the invention refers to a compound of formula (I), wherein R₄ is H or selected from the group consisting of halogen, —OR₇, —SO₂R₈, —(C₁-C₆)alkyl, —(C₁-C₆)haloalkyl, —(C₁-C₆)aminoalkyl, —(C₁-C₆)hydroxyalkyl, —(C₁-C₆)alkoxy, and wherein any of such —(C₁-C₆)alkyl, —(C₁-C₆)haloalkyl, —(C₁-C₆)alkoxy may be optionally substituted by one or more groups selected from halogen, —C(O)NR₉R₁₀, —NR₉—C(O)R₁₀, aryl, —(C₁-C₆)cycloalkyl and heterocycloalkyl.

In another preferred embodiment, the invention refers to a compound of formula (I), wherein R₅ and R₆ are H.

In another preferred embodiment, the invention refers to a compound of formula (I), wherein B is a ring selected from aryl or heteroaryl, said aryl or heteroaryl being optionally substituted with one or more substituent selected from oxo, cyano, —(C₁-C₆)alkyl.

In another preferred embodiment, the invention refers to a compound of formula (I), wherein B is heteroaryl, said heteroaryl may be fused to second saturated or unsaturated ring optionally containing one or more heteroatoms selected from N and O to form a tricyclic or spiro tricyclic ring system, said heteroaryl being optionally substituted with one or more substituent selected from —(C₁-C₆)alkyl and heterocycloalkyl.

In a preferred embodiment, the invention refers to a compound of formula (I), wherein A is aryl, and such aryl may be optionally substituted by one or more halogen, —(C₁-C₆)alkyl, —(C₁-C₆)haloalkyl, —(C₁-C₆)hydroxyalkyl and —(C₁-C₆)amminoalkyl. In another preferred embodiment the invention refers to a compound of formula (I), wherein A is pyridine which may be optionally substituted by one or more halogen, —(C₁-C₆)alkyl, —(C₁-C₆)haloalkyl, —(C₁-C₆)hydroxyalkyl and —(C₁-C₆)amminoalkyl.

In more preferred embodiment, the invention refers to a compound of formula (I), wherein
R₁ is H or selected from the group consisting of —(C₁-C₆)alkyl, heterocycloalkyl or heteroaryl, wherein said —(C₁-C₆)alkyl may be optionally substituted by one or more groups selected from aryl, —(C₃-C₆)cycloalkyl, —C(O)NR₉R₁₀, heterocycloalkyl and heteroaryl;
R₂ and R₃ are independently H or —(C₁-C₆)alkyl;
R₄ is H or selected from the group consisting of halogen, —OR₇, —SR₈, —SO₂R₈, —(C₁-C₆)alkyl, —(C₁-C₆)haloalkyl, —(C₁-C₆)hydroxyalkyl, —(C₁-C₆)alkoxy, wherein any of such —(C₁-C₆)alkyl may be optionally substituted by one or more groups selected from aryl, —(C₁-C₆)cycloalkyl and heterocycloalkyl;
R₅ and R₆ are H;
A is aryl or pyridine, wherein such aryl or pyridine may be optionally substituted by one or more halogen, —(C₁-C₆)alkyl, —(C₁-C₆)haloalkyl, —(C₁-C₆)hydroxyalkyl and —(C₁-C₆)amminoalkyl;
B is a ring selected from optionally substituted aryl or heteroaryl, said heteroaryl selected from the group consisting of

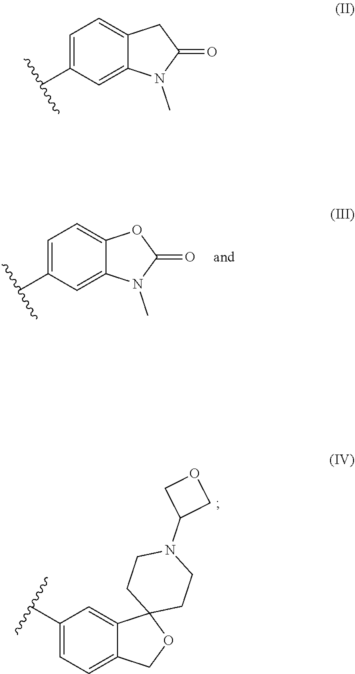 [SMILES: *c1ccc2c(c1)N(C)C(=O)C2 ; *c1ccc2oc(=O)n(C)c2c1 ; *c1ccc2c(c1)C1(CCN(C3COC3)CC1)OC2]

and
R₇, R₈, R₉, R₁₀, are defined as above.

In a preferred embodiment, the invention refers to a compound of formula (I), wherein
R₄ is selected from the group consisting of halogen, —OR₇, —SR₈, —SO₂R₈, —(C₁-C₆)alkyl, —(C₁-C₆)haloalkyl, —(C₁-C₆)aminoalkyl, —(C₁-C₆)hydroxyalkyl, —(C₁-C₆)alkoxy, —(C₁-C₆)alkyl-S—(C₁-C₄)alkyl, heterocycloalkyl, aryl, heteroaryl, —(C₃-C₆)cycloalkyl, wherein any of such —(C₁-C₆)alkyl, —(C₁-C₆)haloalkyl, —(C₁-C₆)alkoxy, —(C₁-C₆)alkyl-S—(C₁-C₄)alkyl may be optionally substituted by one or more groups selected from halogen, —NR₉SO₂R₁₀, —SO₂NR₉R₁₀, —C(O)NR₉R₁₀, —NR₉R₁₀, —NR₉—C(O)R₁₀, aryl, —(C₃-C₆)cycloalkyl, heterocycloalkyl and heteroaryl;
and
R₁, R₂, R₃, R₅, R₆, R₇, R₈, R₉, R₁₀, and cycle A and B are defined as above.

All the listed groups for each of the variable moieties R₁, R₂, R₃, R₄, R₅, R₆, R₇, R₈, R₉, R₁₀, A and B of the compounds of the invention have to be intended as alternatives and may be combined with each other in embodiments which are included in the scope of the invention.

In a more preferred embodiment, the invention refers to at least one of the compounds listed in the Table 1 below and pharmaceutical acceptable salts thereof.

TABLE 1
List of preferred compounds

| Example | | |
|---|---|---|
| N. | Structure | Iupac Name |
| 1 | 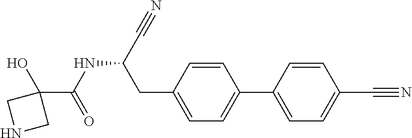 [SMILES: N#Cc1ccc(-c2ccc(C[C@@H](C#N)NC(=O)C3(O)CNC3)cc2)cc1] | (S)-N-(1-cyano-2-(4′-cyano- [1,1′-biphenyl]-4-yl)ethyl)-3- hydroxyazetidine-3-carboxamide |
| 2 | 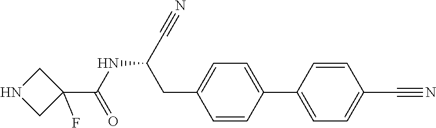 [SMILES: N#Cc1ccc(-c2ccc(C[C@@H](C#N)NC(=O)C3(F)CNC3)cc2)cc1] | (S)-N-(1-cyano-2-(4′-cyano- [1,1′-biphenyl]-4-yl)ethyl)-3- fluoroazetidine-3-carboxamide |
| 3 | 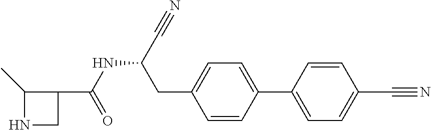 [SMILES: CC1NCC1C(=O)N[C@H](C#N)Cc1ccc(-c2ccc(C#N)cc2)cc1] | N-((S)-1-cyano-2-(4′-cyano- [1,1′-biphenyl]-4-yl)ethyl)-2- methylazetidine-3-carboxamide |
| 4 | 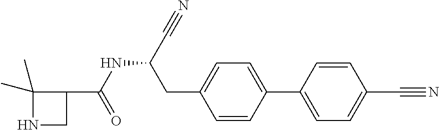 [SMILES: CC1(C)NCC1C(=O)N[C@H](C#N)Cc1ccc(-c2ccc(C#N)cc2)cc1] | N-((S)-1-cyano-2-(4′-cyano- [1,1′-biphenyl]-4-yl)ethyl)-2,2- dimethylazetidine-3- carboxamide |
| 5 | 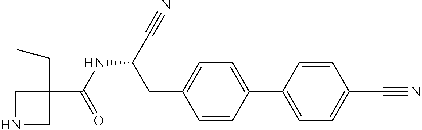 [SMILES: CCC1(C(=O)N[C@H](C#N)Cc2ccc(-c3ccc(C#N)cc3)cc2)CNC1] | (S)-N-(1-cyano-2-(4′-cyano- [1,1′-biphenyl]-4-yl)ethyl)-3- ethylazetidine-3-carboxamide |
| 6 | 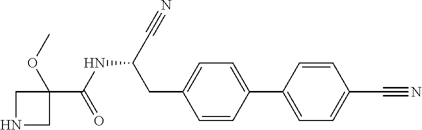 [SMILES: COC1(C(=O)N[C@H](C#N)Cc2ccc(-c3ccc(C#N)cc3)cc2)CNC1] | (S)-N-(1-cyano-2-(4′-cyano- [1,1′-biphenyl]-4-yl)ethyl)-3- methoxyazetidine-3- carboxamide |
| 7 | 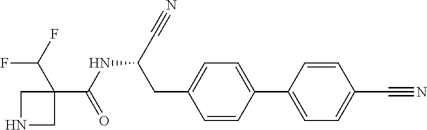 [SMILES: N#Cc1ccc(-c2ccc(C[C@@H](C#N)NC(=O)C3(C(F)F)CNC3)cc2)cc1] | (S)-N-(1-cyano-2-(4′-cyano- [1,1′-biphenyl]-4-yl)ethyl)-3- (difluoromethyl)azetidine-3- carboxamide |
| 8 | 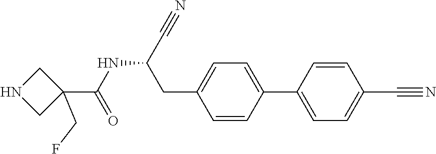 [SMILES: N#Cc1ccc(-c2ccc(C[C@@H](C#N)NC(=O)C3(CF)CNC3)cc2)cc1] | (S)-N-(1-cyano-2-(4′-cyano- [1,1′-biphenyl]-4-yl)ethyl)-3- (fluoromethyl)azetidine-3- carboxamide |
| 9 | 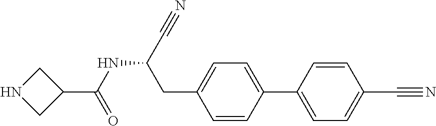 [SMILES: N#Cc1ccc(-c2ccc(C[C@@H](C#N)NC(=O)C3CNC3)cc2)cc1] | (S)-N-(1-cyano-2-(4′-cyano- [1,1′-biphenyl]-4- yl)ethyl)azetidine-3- carboxamide |
| 10 | 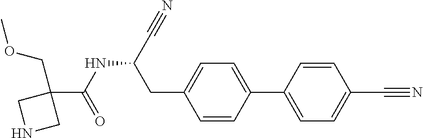 [SMILES: COCC1(C(=O)N[C@H](C#N)Cc2ccc(-c3ccc(C#N)cc3)cc2)CNC1] | (S)-N-(1-cyano-2-(4′-cyano- [1,1′-biphenyl]-4-yl)ethyl)-3- (methoxymethyl)azetidine-3- carboxamide |
| 11 | 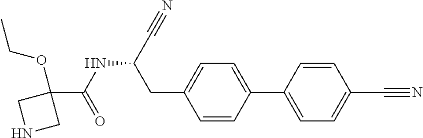 [SMILES: CCOC1(C(=O)N[C@H](C#N)Cc2ccc(-c3ccc(C#N)cc3)cc2)CNC1] | (S)-N-(1-cyano-2-(4′-cyano- [1,1′-biphenyl]-4-yl)ethyl)-3- ethoxyazetidine-3-carboxamide |
| 12 | 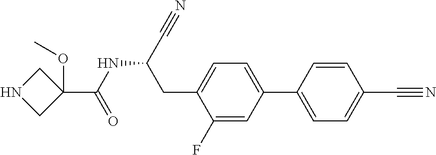 [SMILES: COC1(C(=O)N[C@H](C#N)Cc2ccc(-c3ccc(C#N)cc3)cc2F)CNC1] | (S)-N-(1-cyano-2-(4′-cyano-3- fluoro-[1,1′-biphenyl]-4- yl)ethyl)-3-methoxyazetidine-3- carboxamide |
| 13 | 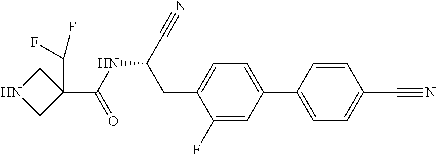 [SMILES: N#Cc1ccc(-c2ccc(C[C@@H](C#N)NC(=O)C3(C(F)F)CNC3)c(F)c2)cc1] | (S)-N-(1-cyano-2-(4′-cyano-3- fluoro-[1,1′-biphenyl]-4- yl)ethyl)-3- (difluoromethyl)azetidine-3- carboxamide |
| 14 | 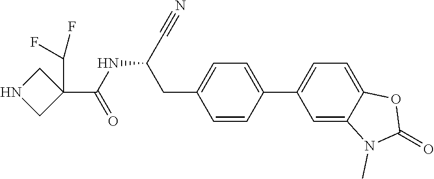 [SMILES: Cn1c(=O)oc2ccc(-c3ccc(C[C@@H](C#N)NC(=O)C4(C(F)F)CNC4)cc3)cc21] | (S)-N-(1-cyano-2-(4-(3-methyl- 2-oxo-2,3- dihydrobenzo[d]oxazol-5- yl)phenyl)ethyl)-3- (difluoromethyl)azetidine-3- carboxamide |
| 15 | 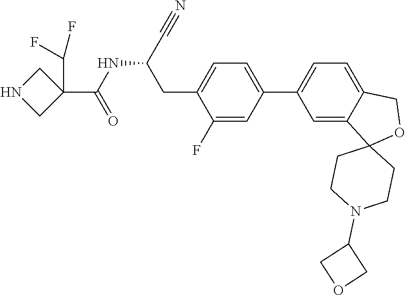 [SMILES: N#C[C@H](Cc1ccc(-c2ccc3c(c2)C2(CCN(C4COC4)CC2)OC3)cc1F)NC(=O)C1(C(F)F)CNC1] | (S)-N-(1-cyano-2-(2-fluoro-4- (1′-(oxetan-3-yl)-3H- spiro[isobenzofuran-1,4′- piperidin]-6-yl)phenyl)ethyl)-3- (difluoromethyl)azetidine-3- carboxamide |
| 16 | 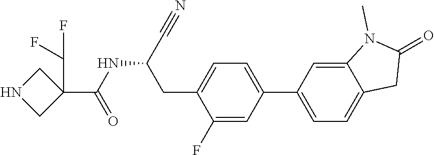 [SMILES: CN1C(=O)Cc2ccc(-c3ccc(C[C@@H](C#N)NC(=O)C4(C(F)F)CNC4)c(F)c3)cc21] | (S)-N-(1-cyano-2-(2-fluoro-4-(1- methyl-2-oxoindolin-6- yl)phenyl)ethyl)-3- (difluoromethyl)azetidine-3- carboxamide |
| 17 | 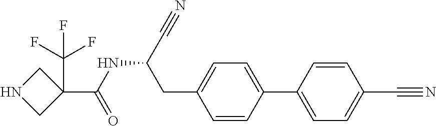 [SMILES: N#Cc1ccc(-c2ccc(C[C@@H](C#N)NC(=O)C3(C(F)(F)F)CNC3)cc2)cc1] | (S)-N-(1-cyano-2-(4′-cyano- [1,1′-biphenyl]-4-yl)ethyl)-3- (trifluoromethyl)azetidine-3- carboxamide |
| 18 | 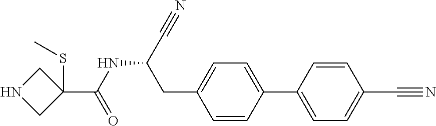 [SMILES: CSC1(C(=O)N[C@H](C#N)Cc2ccc(-c3ccc(C#N)cc3)cc2)CNC1] | (S)-N-(1-cyano-2-(4′-cyano- [1,1′-biphenyl]-4-yl)ethyl)-3- (methylthio)azetidine-3- carboxamide |
| 19 | 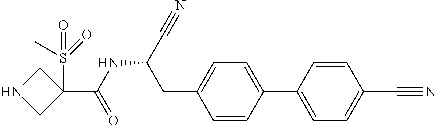 [SMILES: CS(=O)(=O)C1(C(=O)N[C@H](C#N)Cc2ccc(-c3ccc(C#N)cc3)cc2)CNC1] | (S)-N-(1-cyano-2-(4′-cyano- [1,1′-biphenyl]-4-yl)ethyl)-3- (methylsulfonyl)azetidine-3- carboxamide |
| 20 | 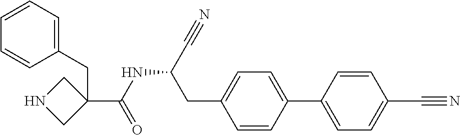 [SMILES: N#Cc1ccc(-c2ccc(C[C@@H](C#N)NC(=O)C3(Cc4ccccc4)CNC3)cc2)cc1] | (S)-3-benzyl-N-(1-cyano-2-(4′- cyano-[1,1′-biphenyl]-4- yl)ethyl)azetidine-3- carboxamide |
| 21 | 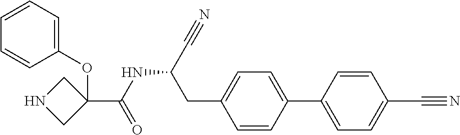 [SMILES: N#Cc1ccc(-c2ccc(C[C@@H](C#N)NC(=O)C3(Oc4ccccc4)CNC3)cc2)cc1] | (S)-N-(1-cyano-2-(4′-cyano- [1,1′-biphenyl]-4-yl)ethyl)-3- phenoxyazetidine-3- carboxamide |
| 22 | 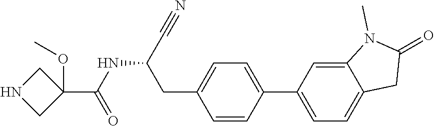 [SMILES: COC1(C(=O)N[C@H](C#N)Cc2ccc(-c3ccc4c(c3)N(C)C(=O)C4)cc2)CNC1] | (S)-N-(1-cyano-2-(4-(1-methyl- 2-oxoindolin-6-yl)phenyl)ethyl)- 3-methoxyazetidine-3- carboxamide |
| 23 | 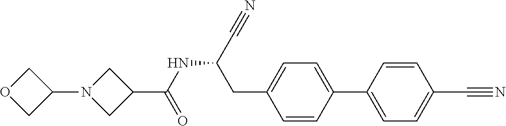 [SMILES: N#Cc1ccc(-c2ccc(C[C@@H](C#N)NC(=O)C3CN(C4COC4)C3)cc2)cc1] | (S)-N-(1-cyano-2-(4′-cyano- [1,1′-biphenyl]-4-yl)ethyl)-1- (oxetan-3-yl)azetidine-3- carboxamide |
| 24 | 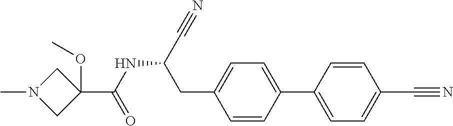 [SMILES: COC1(C(=O)N[C@H](C#N)Cc2ccc(-c3ccc(C#N)cc3)cc2)CN(C)C1] | (S)-N-(1-cyano-2-(4′-cyano- [1,1′-biphenyl]-4-yl)ethyl)-3- methoxy-1-methylazetidine-3- carboxamide |
| 25 | 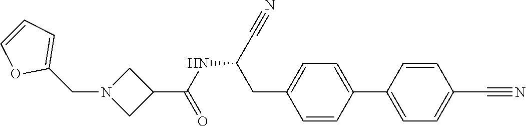 [SMILES: N#Cc1ccc(-c2ccc(C[C@@H](C#N)NC(=O)C3CN(Cc4ccco4)C3)cc2)cc1] | (S)-N-(1-cyano-2-(4′-cyano- [1,1′-biphenyl]-4-yl)ethyl)-1- (furan-2-ylmethyl)azetidine-3- carboxamide |
| 26 | 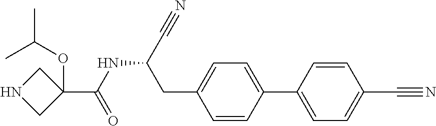 [SMILES: CC(C)OC1(C(=O)N[C@H](C#N)Cc2ccc(-c3ccc(C#N)cc3)cc2)CNC1] | (S)-N-(1-cyano-2-(4′-cyano- [1,1′-biphenyl]-4-yl)ethyl)-3- isopropoxyazetidine-3- carboxamide |
| 27 | 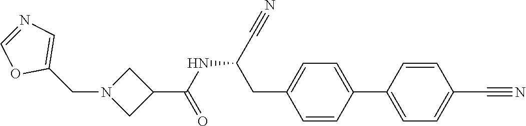 [SMILES: N#Cc1ccc(-c2ccc(C[C@@H](C#N)NC(=O)C3CN(Cc4cnco4)C3)cc2)cc1] | (S)-N-(1-cyano-2-(4′-cyano- [1,1′-biphenyl]-4-yl)ethyl)-1- (oxazol-5-ylmethyl)azetidine-3- carboxamide |
| 28 | 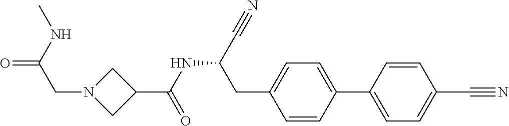 [SMILES: CNC(=O)CN1CC(C(=O)N[C@H](C#N)Cc2ccc(-c3ccc(C#N)cc3)cc2)C1] | (S)-N-(1-cyano-2-(4′-cyano- [1,1′-biphenyl]-4-yl)ethyl)-1-(2- (methylamino)-2- oxoethyl)azetidine-3- carboxamide |
| 29 | 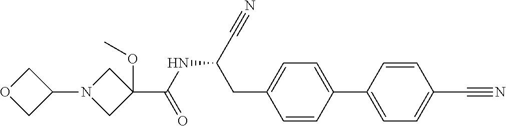 [SMILES: COC1(C(=O)N[C@H](C#N)Cc2ccc(-c3ccc(C#N)cc3)cc2)CN(C2COC2)C1] | (S)-N-(1-cyano-2-(4′-cyano- [1,1′-biphenyl]-4-yl)ethyl)-3- methoxy-1-(oxetan-3- yl)azetidine-3-carboxamide |
| 30 | 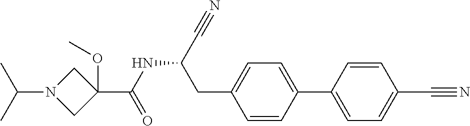 [SMILES: COC1(C(=O)N[C@H](C#N)Cc2ccc(-c3ccc(C#N)cc3)cc2)CN(C(C)C)C1] | (S)-N-(1-cyano-2-(4′-cyano- [1,1′-biphenyl]-4-yl)ethyl)-1- isopropyl-3-methoxyazetidine-3- carboxamide |
| 31 | 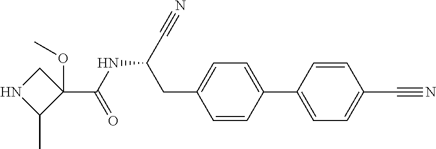 [SMILES: COC1(C(=O)N[C@H](C#N)Cc2ccc(-c3ccc(C#N)cc3)cc2)CNC1C] | N-((S)-1-cyano-2-(4′-cyano- [1,1′-biphenyl]-4-yl)ethyl)-3- methoxy-2-methylazetidine-3- carboxamide |
| 32 | 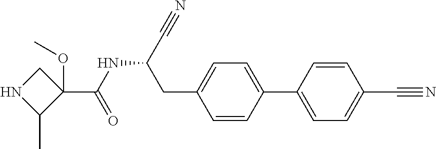 [SMILES: COC1(C(=O)N[C@H](C#N)Cc2ccc(-c3ccc(C#N)cc3)cc2)CNC1C] | diastereoisomer 1 of N-((S)-1- cyano-2-(4′-cyano-[1,1′- biphenyl]-4-yl)ethyl)-3- methoxy-2-methylazetidine-3- carboxamide |
| 33 | 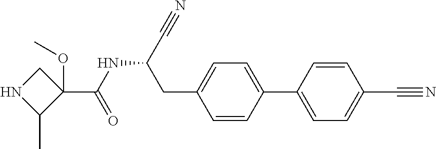 [SMILES: COC1(C(=O)N[C@H](C#N)Cc2ccc(-c3ccc(C#N)cc3)cc2)CNC1C] | mixture of diastereosomer 2 and 3 of N-((S)-1-cyano-2-(4′-cyano- [1,1′-biphenyl]-4-yl)ethyl)-3- methoxy-2-methylazetidine-3- carboxamide |
| 34 | 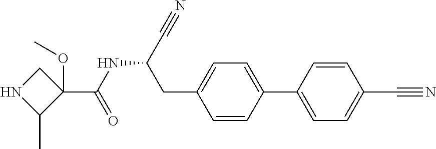 [SMILES: COC1(C(=O)N[C@H](C#N)Cc2ccc(-c3ccc(C#N)cc3)cc2)CNC1C] | diastereoisomer 4 of N-((S)-1- cyano-2-(4′-cyano-[1,1′- biphenyl]-4-yl)ethyl)-3- methoxy-2-methylazetidine-3- carboxamide |
| 35 | 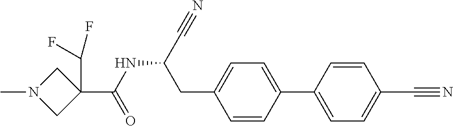 [SMILES: CN1CC(C(=O)N[C@H](C#N)Cc2ccc(-c3ccc(C#N)cc3)cc2)(C(F)F)C1] | (S)-N-(1-cyano-2-(4′-cyano- [1,1′-biphenyl]-4-yl)ethyl)-3- (difluoromethyl)-1- methylazetidine-3-carboxamide |
| 36 | 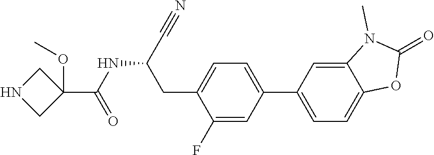 [SMILES: COC1(C(=O)N[C@H](C#N)Cc2ccc(-c3ccc4oc(=O)n(C)c4c3)cc2F)CNC1] | (S)-N-(1-cyano-2-(2-fluoro-4-(3- methyl-2-oxo-2,3- dihydrobenzo[d]oxazol-5- yl)phenyl)ethyl)-3- methoxyazetidine-3- carboxamide |
| 37 | 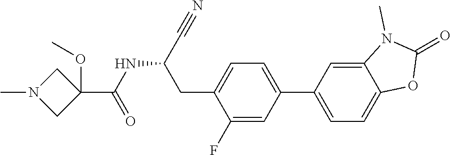 [SMILES: COC1(C(=O)N[C@H](C#N)Cc2ccc(-c3ccc4oc(=O)n(C)c4c3)cc2F)CN(C)C1] | (S)-N-(1-cyano-2-(2-fluoro-4-(3- methyl-2-oxo-2,3- dihydrobenzo[d]oxazol-5- yl)phenyl)ethyl)-3-methoxy-1- methylazetidine-3-carboxamide |
| 38 | 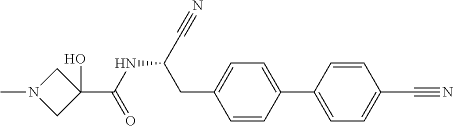 [SMILES: CN1CC(O)(C(=O)N[C@H](C#N)Cc2ccc(-c3ccc(C#N)cc3)cc2)C1] | (S)-N-(1-cyano-2-(4′-cyano- [1,1′-biphenyl]-4-yl)ethyl)-3- hydroxy-1-methylazetidine-3- carboxamide |
| 39 | 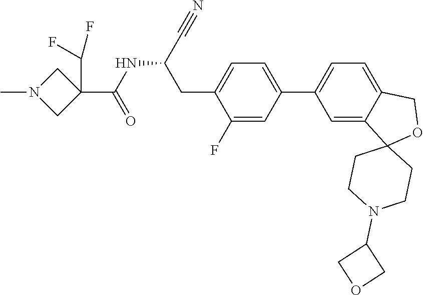 [SMILES: CN1CC(C(=O)N[C@H](C#N)Cc2ccc(-c3ccc4c(c3)C3(CCN(C5COC5)CC3)OC4)cc2F)(C(F)F)C1] | (S)-N-(1-cyano-2-(2-fluoro-4- (1′-(oxetan-3-yl)-3H- spiro[isobenzofuran-1,4′- piperidin]-6-yl)phenyl)ethyl)-3- (difluoromethyl)-1- methylazetidine-3-carboxamide |
| 40 | 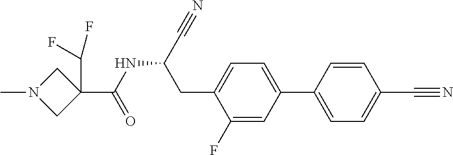 [SMILES: CN1CC(C(=O)N[C@H](C#N)Cc2ccc(-c3ccc(C#N)cc3)cc2F)(C(F)F)C1] | (S)-N-(1-cyano-2-(4′-cyano-3- fluoro-[1,1′-biphenyl]-4- yl)ethyl)-3-(difluoromethyl)-1- methylazetidine-3-carboxamide |
| 41 | 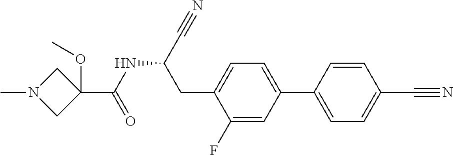 [SMILES: COC1(C(=O)N[C@H](C#N)Cc2ccc(-c3ccc(C#N)cc3)cc2F)CN(C)C1] | (S)-N-(1-cyano-2-(4′-cyano-3- fluoro-[1,1′-biphenyl]-4- yl)ethyl)-3-methoxy-1- methylazetidine-3-carboxamide |
| 42 | 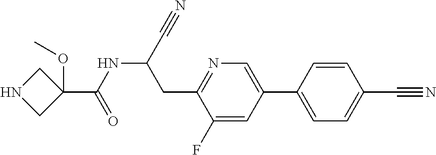 [SMILES: COC1(C(=O)NC(C#N)Cc2ncc(-c3ccc(C#N)cc3)cc2F)CNC1] | N-(1-cyano-2-(5-(4- cyanophenyl)-3-fluoropyridin-2- yl)ethyl)-3-methoxyazetidine-3- carboxamide |
| 43 | 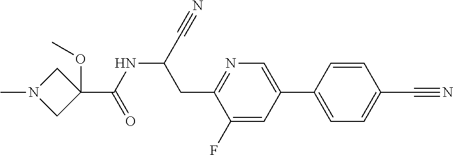 [SMILES: COC1(C(=O)NC(C#N)Cc2ncc(-c3ccc(C#N)cc3)cc2F)CN(C)C1] | N-(1-cyano-2-(5-(4- cyanophenyl)-3-fluoropyridin-2- yl)ethyl)-3-methoxy-1- methylazetidine-3-carboxamide |
| 44 | 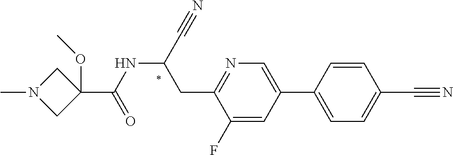 [SMILES: COC1(C(=O)NC(C#N)Cc2ncc(-c3ccc(C#N)cc3)cc2F)CN(C)C1] | enantiomer 1 of N-(1-cyano-2- (5-(4-cyanophenyl)-3- fluoropyridin-2-yl)ethyl)-3- methoxy-1-methylazetidine-3- carboxamide |
| 45 | 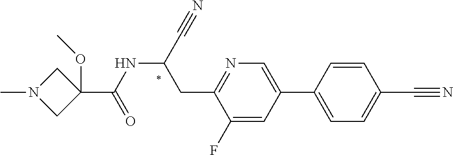 [SMILES: COC1(C(=O)NC(C#N)Cc2ncc(-c3ccc(C#N)cc3)cc2F)CN(C)C1] | Enantiomer 2 of N-(1-cyano-2- (5-(4-cyanophenyl)-3- fluoropyridin-2-yl)ethyl)-3- methoxy-1-methylazetidine-3- carboxamide |
| 46 | 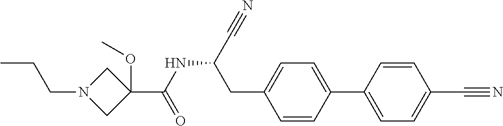 [SMILES: CCCN1CC(OC)(C(=O)N[C@H](C#N)Cc2ccc(-c3ccc(C#N)cc3)cc2)C1] | (S)-N-(1-cyano-2-(4′-cyano- [1,1′-biphenyl]-4-yl)ethyl)-3- methoxy-1-propylazetidine-3- carboxamide |
| 47 | 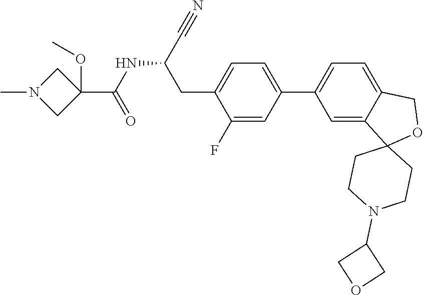 [SMILES: COC1(C(=O)N[C@H](C#N)Cc2ccc(-c3ccc4c(c3)C3(CCN(C5COC5)CC3)OC4)cc2F)CN(C)C1] | (S)-N-(1-cyano-2-(2-fluoro-4- (1′-(oxetan-3-yl)-3H- spiro[isobenzofuran-1,4′- piperidin]-6-yl)phenyl)ethyl)-3- methoxy-1-methylazetidine-3- carboxamide |
| 48 | 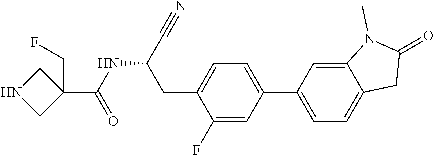 [SMILES: CN1C(=O)Cc2ccc(-c3ccc(C[C@@H](C#N)NC(=O)C4(CF)CNC4)c(F)c3)cc21] | (S)-N-(1-cyano-2-(2-fluoro-4-(1- methyl-2-oxoindolin-6- yl)phenyl)ethyl)-3- (fluoromethyl)azetidine-3- carboxamide |
| 49 | 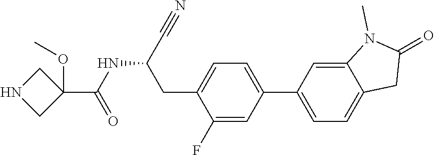 [SMILES: COC1(C(=O)N[C@H](C#N)Cc2ccc(-c3ccc4c(c3)N(C)C(=O)C4)cc2F)CNC1] | (S)-N-(1-cyano-2-(2-fluoro-4-(1- methyl-2-oxoindolin-6- yl)phenyl)ethyl)-3- methoxyazetidine-3- carboxamide |
| 50 | 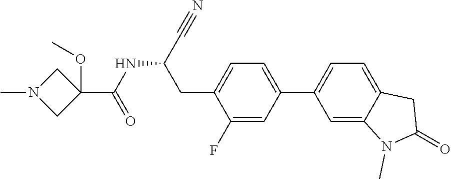 [SMILES: COC1(C(=O)N[C@H](C#N)Cc2ccc(-c3ccc4c(c3)N(C)C(=O)C4)cc2F)CN(C)C1] | (S)-N-(1-cyano-2-(2-fluoro-4-(1- methyl-2-oxoindolin-6- yl)phenyl)ethyl)-3-methoxy-1- methylazetidine-3-carboxamide |
| 51 | 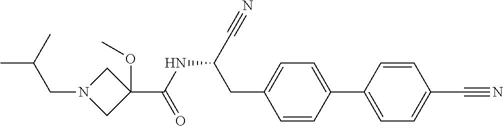 [SMILES: COC1(C(=O)N[C@H](C#N)Cc2ccc(-c3ccc(C#N)cc3)cc2)CN(CC(C)C)C1] | (S)-N-(1-cyano-2-(4′-cyano- [1,1′-biphenyl]-4-yl)ethyl)-1- isobutyl-3-methoxyazetidine-3- carboxamide |
| 52 | 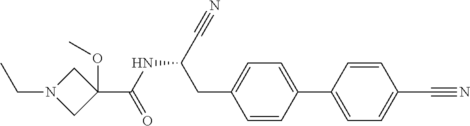 [SMILES: CCN1CC(OC)(C(=O)N[C@H](C#N)Cc2ccc(-c3ccc(C#N)cc3)cc2)C1] | (S)-N-(1-cyano-2-(4′-cyano- [1,1′-biphenyl]-4-yl)ethyl)-1- ethyl-3-methoxyazetidine-3- carboxamide |
| 53 | 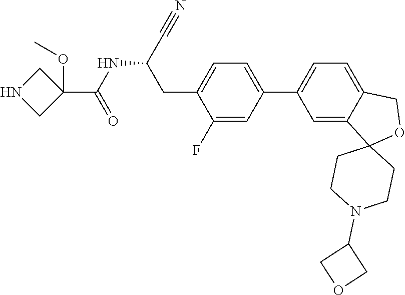 [SMILES: COC1(C(=O)N[C@H](C#N)Cc2ccc(-c3ccc4c(c3)C3(CCN(C5COC5)CC3)OC4)cc2F)CNC1] | (S)-N-(1-cyano-2-(2-fluoro-4- (1′-(oxetan-3-yl)-3H- spiro[isobenzofuran-1,4′- piperidin]-6-yl)phenyl)ethyl)-3- methoxyazetidine-3- carboxamide |

It is to be understood that all the single deuterated forms, enantiomers, diastereoisomers and mixtures thereof, in any proportion, of the compounds of formula (I) of the invention are encompassed within the scope of the present invention.

In a preferred embodiment, the invention refers to a compound of formula (I) as DPP1 antagonist.

In this respect, it has now been found that the compounds of formula (I) of the present invention have an inhibitor drug potency expressed as negative logarithmic value of half maximal inhibitory concentration in molar concentration (pIC50) on receptors DPP1 higher than the value 5. Preferably, the compounds of the present invention have an pIC₅₀ on DPP1 between the value 5 and 6 (+).

Preferably, the compounds of the present invention have an pIC₅₀ on DPP1 equal or higher than 6. Preferably, the compounds of the present invention have an pIC₅₀ on DPP1 between the value 6 and 7 (++).

More preferably, the compounds of the present invention have an pIC50 on DPP1 between the value 7 and 8 (+++).

Even more preferably, the compounds of the present invention have an pIC50 on DPP1 equal or higher than 8 (+++).

The present invention also refers to a pharmaceutical composition comprising a compound of formula (I) or a pharmaceutically acceptable salt thereof, in admixture with at least one or more pharmaceutically acceptable carrier and/or excipient.

As used herein, “safe and effective amount” in reference to a compound of formula (I) or a pharmaceutically acceptable salt thereof or other pharmaceutically active agent means an amount of the compound sufficient to treat the patient's condition but low enough to avoid serious side effects and it can nevertheless be routinely determined by the skilled artisan.

The compounds of formula (I) may be administered once or according to a dosing regimen wherein a number of doses are administered at varying intervals of time for a given period of time. Typical daily dosages may vary depending upon the route of administration chosen.

In one embodiment, the invention refers to a pharmaceutical composition of compounds of formula (I) in admixture with one or more pharmaceutically acceptable carrier or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation.

Preferably, the compounds of the present invention are administered orally or by inhalation.

In a more preferred embodiment, the compounds of the present invention or their pharmaceutical compositions are administered orally.

In one preferred embodiment, the pharmaceutical composition comprising the compounds of formula (I) is a solid oral dosage form such as tablets, gel caps, capsules, caplets, granules, lozenges and bulk powders.

The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In a further embodiment, the pharmaceutical composition comprising a compound of formula (I) is a liquid oral dosage forms such as aqueous and non-aqueous solutions, emulsions, suspensions, syrups. Such liquid dosage forms can also contain suitable known inert diluents such as water and suitable known excipients such as preservatives, wetting agents, sweeteners, flavorants, as well as agents for emulsifying and/or suspending the compounds of the invention

In a further embodiment, the pharmaceutical composition comprising the compounds of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges, or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers, and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention can be administered as the sole active agent or in combination with other pharmaceutical active ingredients.

The dosages of the compounds of the invention depend upon a variety of factors including among others the particular disease to be treated, the severity of the symptoms, the route of administration and the like.

In a further aspect, the invention refers to the use of the compounds of formula (I) for the preparation of a medicament. In another aspect, the present invention refers to a compound of formula (I) for use as a medicament.

Thus, the invention refers to a compound of formula (I) in the preparation of a medicament, preferably for use in the treatment of disorders associated with DPP1 receptors mechanism.

In a further embodiment, the present invention refers to a compound of formula (I) for use in the treatment of a disease, disorder or condition associated with dysregulation of dipeptidyl peptidase 1 (DPP1).

In one aspect, the invention also refers to a method for the prevention and/or treatment of disorders associated with DPP1 receptors mechanisms, said method comprises administering to a patient in need of such treatment a therapeutically effective amount of a compound of formula (I).

Preferably, the compounds of the present invention are useful for the treatment and/or prevention of inflammatory or obstructive respiratory disease.

More preferably, the compounds of formula (I) of the present invention are useful for the treatment and/or prevention of asthma, chronic obstructive pulmonary disease (COPD), Non-cystic fibrosis bronchiectasis (NCFBE), chronic bronchitis, pneumonia, acute respiratory distress syndrome (ARDS), Acute lung injury (ALI) lung fibrosis, idiopathic pulmonary fibrosis, pulmonary emphysema, smoking-induced emphysema and cystic fibrosis. More preferably, the compounds of formula (I) of the present invention are useful for the treatment and/or prevention of non-cystic fibrosis bronchiectasis (NCFBE).

In one embodiment, the present invention refers to a compound of formula (I) useful for the prevention and/or treatment of inflammatory or obstructive respiratory disease.

In another embodiment, the present invention is directed to the compounds of formula (I) for use for the prevention and/or treatment of an inflammatory or obstructive respiratory disease.

In a further preferred embodiment, the present invention provides a method for preventing and/or treating an inflammatory or obstructive respiratory disease, the method comprising administering a compound of formula (I).

In another aspect, the present invention is directed to a pharmaceutical composition comprising the compounds of formula (I) and one or more pharmaceutically acceptable carriers and/or excipients, for use for the prevention and/or treatment of an inflammatory or obstructive respiratory disease.

In a further aspect, the invention refers to the use of the compounds of formula (I) or its pharmaceutical composition for the preparation of a medicament for the treatment and/or prevention of asthma, chronic obstructive pulmonary disease (COPD), Non-cystic fibrosis bronchiectasis (NCFBE), chronic bronchitis, pneumonia, acute respiratory distress syndrome (ARDS), Acute lung injury (ALI) lung fibrosis, idiopathic pulmonary fibrosis, pulmonary emphysema, smoking-induced emphysema and cystic fibrosis.

In a further aspect, the invention refers to the use of the compounds of formula (I) or its pharmaceutical composition for the preparation of a medicament for the treatment and/or prevention of Non-cystic fibrosis bronchiectasis (NCFBE).

In one embodiment, the invention refers to a compound of formula (I) or a pharmaceutical composition for use in the prevention and/or treatment of an inflammatory or obstructive respiratory disease wherein the inflammatory or obstructive respiratory diseases are selected from: asthma, chronic obstructive pulmonary disease (COPD), Non-cystic fibrosis bronchiectasis (NCFBE), chronic bronchitis, pneumonia, acute respiratory distress syndrome (ARDS), Acute lung injury (ALI), lung fibrosis, idiopathic pulmonary fibrosis, pulmonary emphysema, smoking-induced emphysema and cystic fibrosis.

In preferred embodiment, the present invention provides a method for preventing and/or treating an inflammatory or obstructive respiratory disease, the method comprising administering an effective amount of pharmaceutical composition comprising a compound of formula (I) and one or more pharmaceutically acceptable carriers and/or excipients.

In a further preferred embodiment, the inflammatory or obstructive respiratory diseases mentioned above are selected from asthma, chronic obstructive pulmonary disease (COPD), Non-cystic fibrosis bronchiectasis (NCFBE), chronic bronchitis, pneumonia, acute respiratory distress syndrome (ARDS), Acute lung injury (ALI) lung fibrosis, idiopathic pulmonary fibrosis, pulmonary emphysema, smoking-induced emphysema and cystic fibrosis.

In a further aspect, the invention refers to a method for the treatment and/or prevention of inflammatory or obstructive respiratory diseases mentioned above are selected from asthma, chronic obstructive pulmonary disease (COPD), Non-cystic fibrosis bronchiectasis (NCFBE), chronic bronchitis, pneumonia, acute respiratory distress syndrome (ARDS), Acute lung injury (ALI), lung fibrosis, idiopathic pulmonary fibrosis, pulmonary emphysema, smoking-induced emphysema and cystic fibrosis, the method comprising administering a compound of formula (I).

In a preferred embodiment, the inflammatory or obstructive respiratory diseases mentioned above is Non-cystic fibrosis bronchiectasis (NCFBE).

In a preferred embodiment, the invention refers to a compound of formula (I) or a pharmaceutical composition for use in the prevention and/or treatment of Non-cystic fibrosis bronchiectasis (NCFBE).

In a preferred embodiment, the invention refers to a method for the treatment and/or prevention of Non-cystic fibrosis bronchiectasis (NCFBE), the method comprising administering a compound of formula (I).

In a further aspect, the invention refers to a method for the treatment and/or prevention of inflammatory or obstructive respiratory diseases mentioned above are selected from asthma, chronic obstructive pulmonary disease (COPD), Non-cystic fibrosis bronchiectasis (NCFBE), chronic bronchitis, pneumonia, acute respiratory distress syndrome (ARDS), Acute lung injury (ALI), lung fibrosis, idiopathic pulmonary fibrosis, pulmonary emphysema, smoking-induced emphysema and cystic fibrosis, the method comprising administering an effective amount of pharmaceutical composition comprising a compound of formula (I) and one or more pharmaceutically acceptable carriers and/or excipients.

Any suitable route of administration may be employed for providing a mammal, especially a human, with an effective dosage of the compound of formula (I).

The magnitude of prophylactic or therapeutic dose of the compound of formula (I) will, of course, vary with the nature of the severity of the condition to be treated and with its route of administration, and will generally be determined by clinical trial as required in the pharmaceutical art.

It will also vary according to the age, weight, and response of the individual patient.

In therapeutic use, the compound of formula (I) may be administered by any convenient, suitable or effective route.

All preferred groups or embodiments described above for compounds of formula I may be combined among each other and apply as well mutatis mutandis.

The compounds of the present invention can be prepared in a number of ways known to one skilled in the art of organic synthesis. It will be understood by those skilled in the art of organic synthesis that the functionality present on the molecule should be consistent with the transformation proposed. This will sometimes require a modification of the order of synthetic steps in order to obtain a desired compound of the invention.

While the optimal reaction conditions may vary depending on the particular reactants or solvent used, such conditions can be readily determined by those skilled in the art by routine optimization procedures. Thus, processes described below should not be viewed as limiting the scope of the synthetic methods available for the preparation of the compounds of the invention. In some cases, generally known protective groups (PG) may be employed when needed to mask or protect sensitive or reactive moieties, in accordance to general principles of chemistry (Protective group in organic syntheses, 3rd ed. T. W. Greene, P. G. M. Wuts).

The compounds of formulas (Ia), (Ib) (Ic), (Ie), (If), including all the compounds here above listed, can be generally prepared according to the general synthetic routes outlined in Schemes A-E shown below, using generally known methods or starting from slightly modified reagents, easily identifiable by the skilled person and/or following slightly modified synthetic routes that the skilled person can easily apply.

Each step of the general synthetic routes is performed at an appropriate temperature and for an appropriate time as better detailed in the Experimental Part or applying slightly modified conditions that the skilled person can easily apply.

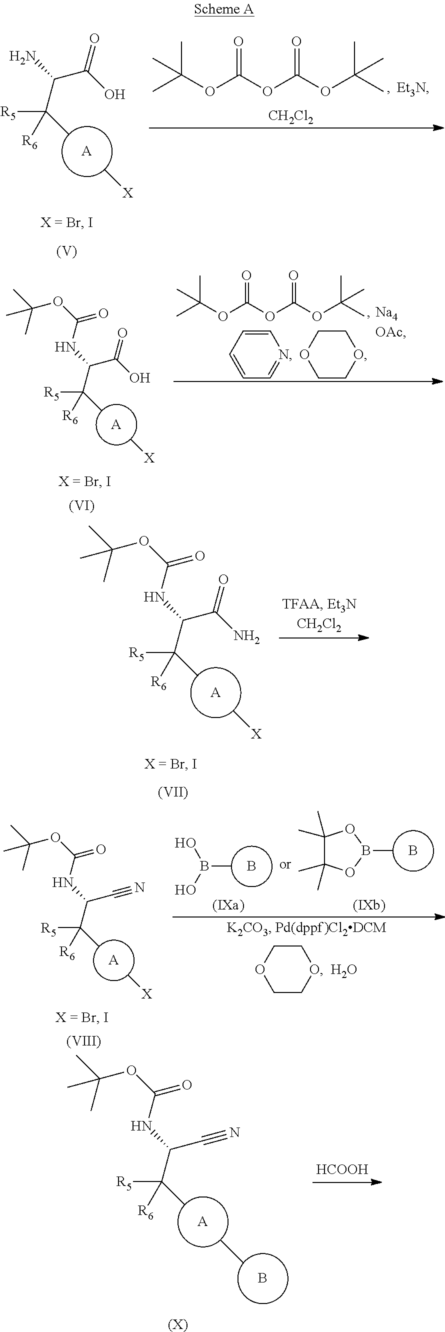

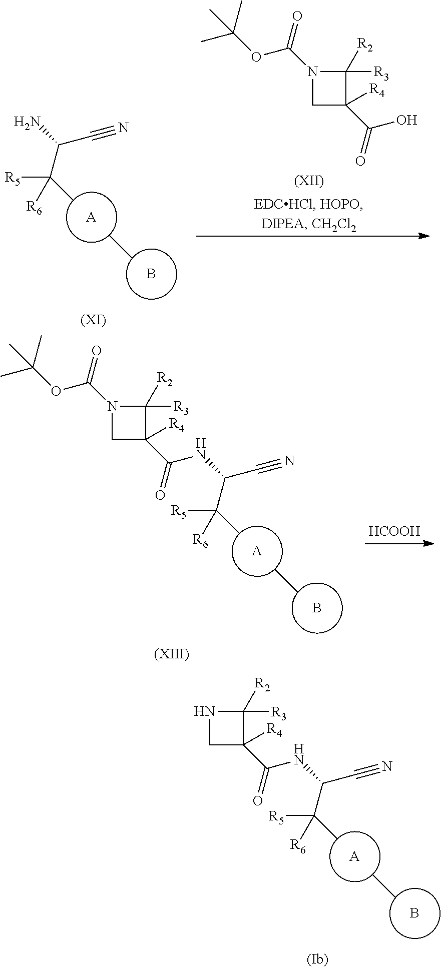

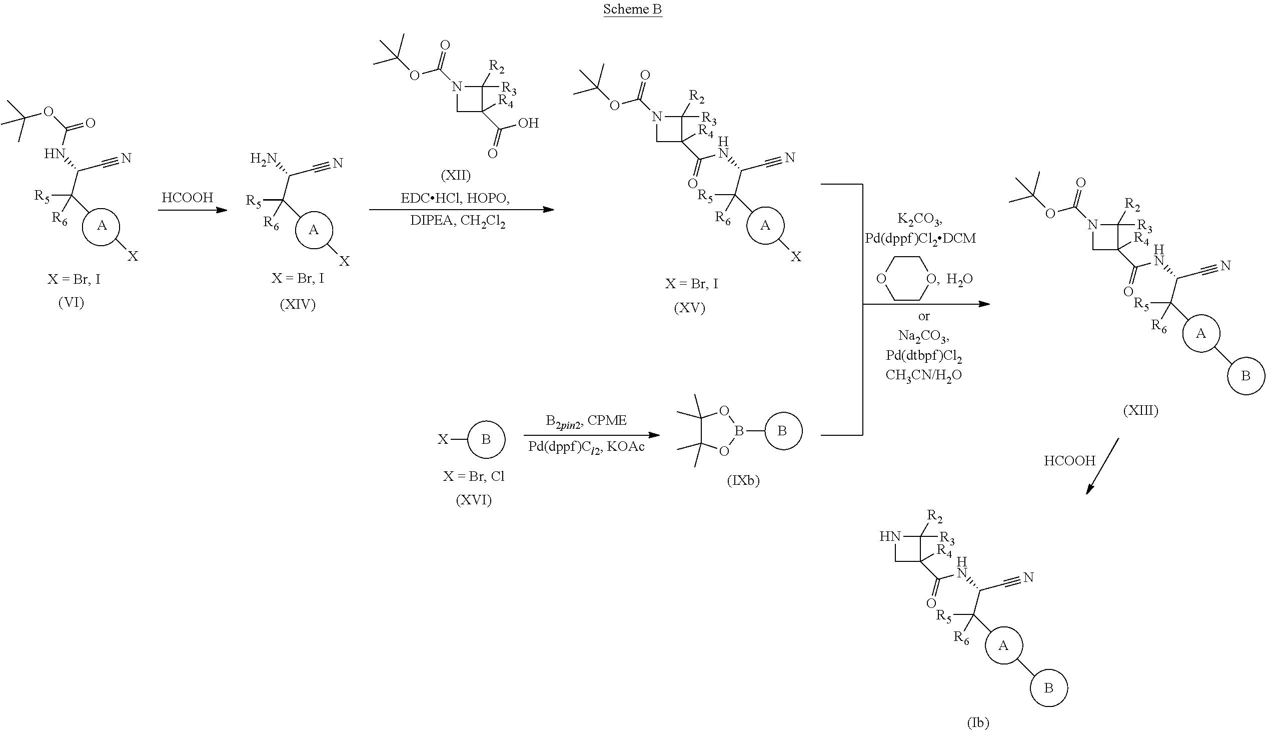

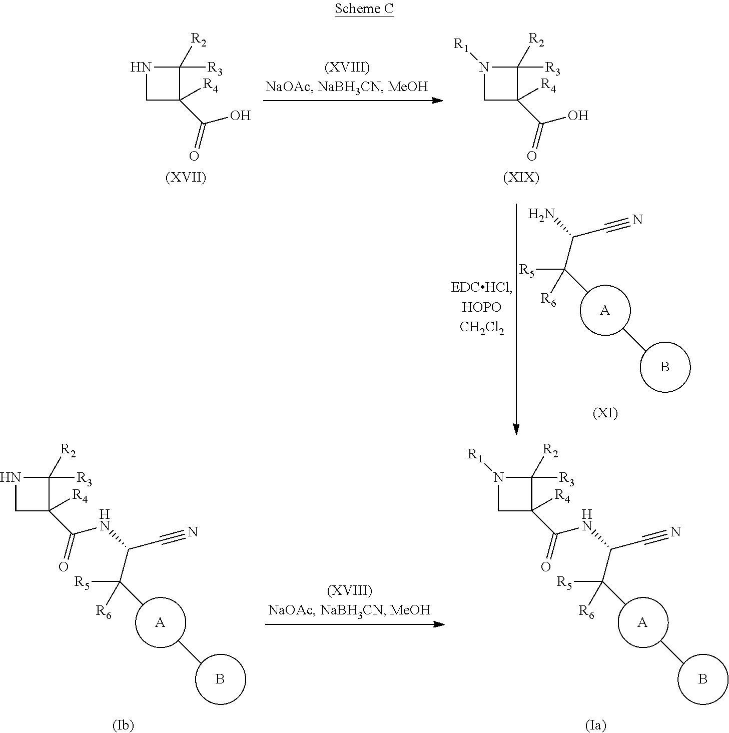

In another embodiment, wherein R₁ is not H, Examples of formula (Ia) can be prepared from azido-acid intermediates (XVII) by reductive amination with suitable ketons or aldehydes (XVIII) obtaining alkylazido acids intermediates (XIX) which were then condensed with aminic intermediates (XI) for obtaining Examples of formula (Ia), according to Scheme C. Alternatively, Examples of formula (Ia) can be prepared by reductive amination with suitable ketons or aldehydes (XVIII) from Examples of formula (Ib), still according to Scheme C.

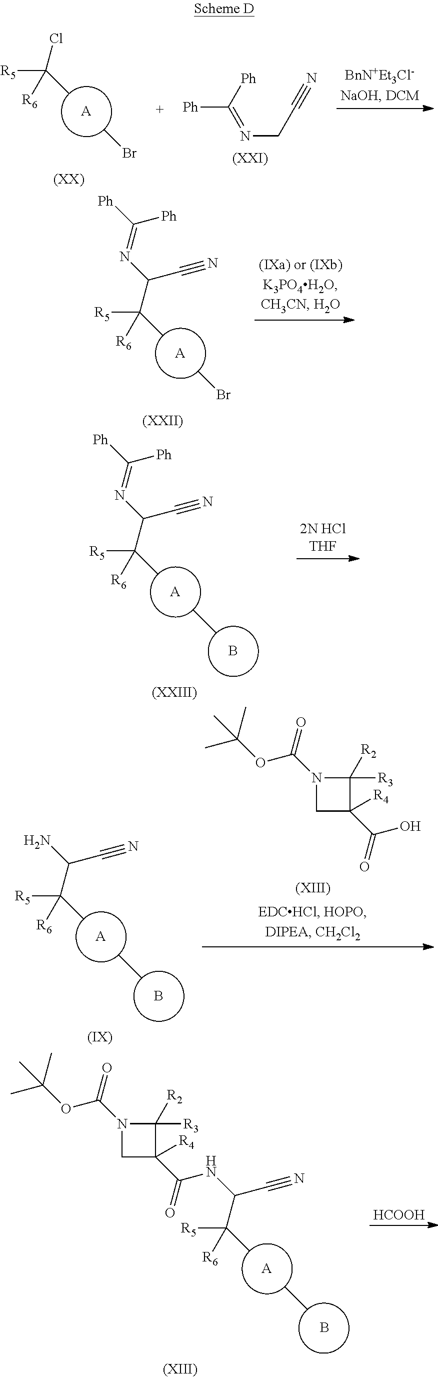

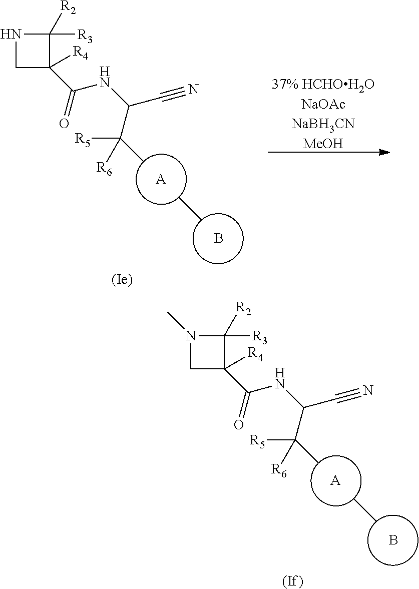

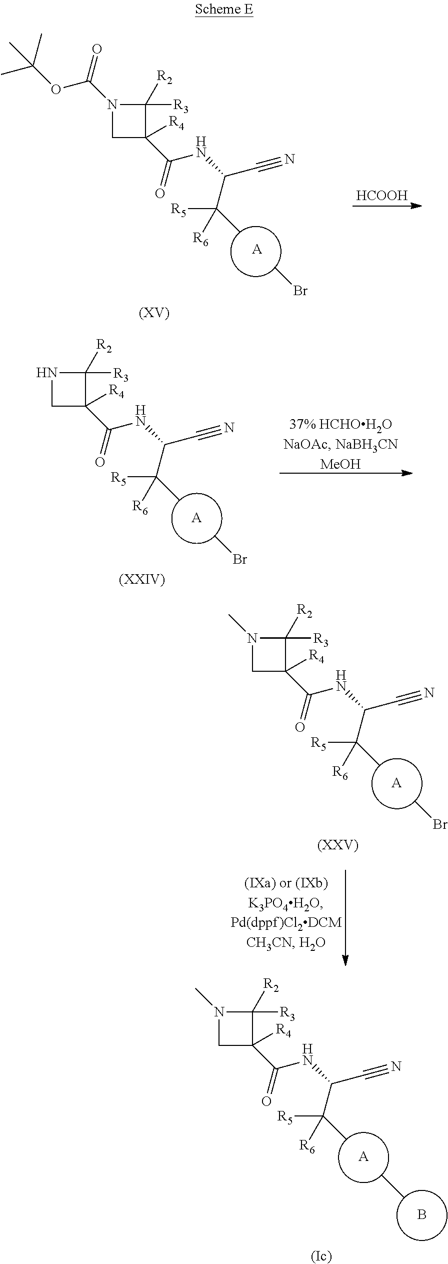

The compounds of the present invention can have one or more asymmetric carbon atoms and can exist in the form of optically pure enantiomers, diastereoisomers or mixtures of diastereoisomers, if not specified otherwise. Both enantiomers of compounds of formula (I) are included in the scope of the present invention. The optically active forms can be obtained for example by resolution of the racemates, by asymmetric synthesis or chiral chromatographic separation or the racemates, by known methods by a person skilled in the art. Separation of racemic mixtures may be achieved by chiral resolution methods, such as chiral chromatographic purification. When the absolute configuration at the stereocenter is not determined, the stereoisomers, or enantiomers, or diastereoisomers, are identified as “1” or “2” and so on, based on the elution order in the chiral chromatographic separation.

### PREPARATIONS OF INTERMEDIATES AND EXAMPLES

Chemical Names of the compounds were generated according to Chem Draw 19.1 Software (Structure to Name).

All reagents, for which the synthesis is not described in the experimental part, are either commercially available, or are known compounds or may be formed from known compounds by known methods by a person skilled in the art.

### Abbreviations

AMC=aminomethyl coumarin; B₂pin₂=bis(pinacolate)diboron; Boc=tert-butoxycarbonyl; CPME=cyclopentyl methyl ether; DBU=1,8-diazabicyclo[5.4.0]undec-7-ene; DCM=dichloromethane; DIPEA=N,N-diisopropylethylamine; DMAP=4-dimethylaminopyridine; DMF=N,N′-dimethylformamide; DMSO=dimethyl sulfoxide; EDC=N-(3-Dimethylaminopropyl)-N′-ethylcarbodiimide; EtOAc=ethyl acetate; EtOH=ethyl alcohol; Ex.=Example; HATU=1-[Bis(dimethylamino)methylene]-1H-1,2,3-triazolo[4,5-b]pyridinium 3-oxide hexafluorophosphate; HOPO=2-hydroxypyridine-N-oxide; HPLC=high pressure liquid chromatography; Int.=Intermediate; LCMS=liquid chromatography/mass spectrometry; MeCN=acetonitrile; MeOH=methyl alcohol; NMR=nuclear magnetic resonance; Pd(dppf)Cl₂=[1,1′-Bis(diphenylphosphino)ferrocene]dichloropalladium(II); Pd(dtbpf)Cl₂=[1,1′-Bis(di-tert-butylphosphino)ferrocene]dichloropalladium(II); PTSA=p-toluenesulfonic acid; RT=room temperature; SFC=supercritical fluid chromatography; TFA=trifluoroacetic acid; TFAA=trifluoroacetic anhydride; TFE=Trifluoroethanol; THE=tetrahydrofuran; UPLC=ultra performance liquid chromatography.

### General Experimental Details and Methods

Instruments, materials and methods employed for analyses

Method AcHSSC18: UPLC-MS was performed on a Waters Acquity I-Class with Waters Diode Array Detector coupled to a Waters SQD2 single quadrapole mass spectrometer using a Waters HSS C18 column (1.8 μm, 100×2.1 mm) being initially held at 5% acetonitrile/water (with 0.1% formic acid in each mobile phase) for 0.4 minutes, followed by a linear gradient of 5-95% within 5.6 minutes and then held at 95% for 0.8 minutes (Flow rate=0.4 mL/min).

Method BicarbBEHC18: UPLC-MS was performed on a Waters Acquity I-Class with Waters Diode Array Detector coupled to a Waters SQD2 single quadrapole mass spectrometer using a Waters BEH Shield RP18 column (1.7 μm, 100×2.1 mm) being initially held at 5% acetonitrile/water (with 10 mM ammonium bicarbonate in each mobile phase) for 0.4 minutes, followed by a linear gradient of 5-95% within 5.6 minutes and then held at 95% for 0.8 minutes (Flow rate=0.4 mL/min).

Method FormicNXC18: HPLC-MS was performed on a Waters Alliance e2695 HPLC with Photodiode Detector 2998 coupled with Column Oven and Mass Spectrometer ZQ in positive and/or negative electron spray ES ionization mode, Phenomenex Gemini NX—C18 150×2 mm, 3 um; gradient: mobile phase solvent A was water for LCMS+0.1% HCOOH, mobile phase solvent B MeCN+0.1% HCOOH; the flow rate was 0.2 mL/min; the column temperature was 30° C. The gradient table was t=0 min 90% A, 10% B, t=12 min 10% A, 90% B, t=30 min 10% A, 90% B, t=31 min 10% A, 90%, UV detection X=215 nm and ES+/ES− range was 50 to 1000 Dalton.

### NMR

¹H Nuclear magnetic resonance (NMR) spectroscopy was carried out using a Bruker instrument operating at 400 MHz using the stated solvent at around room temperature unless otherwise stated. In all cases, NMR data were consistent with the proposed structures. Characteristic chemical shifts (6) are given in parts-per-million using conventional abbreviations for designation of major peaks: e.g. s, singlet; d, doublet; t, triplet; q, quartet; dd, doublet of doublets; dt, doublet of triplets; m, multiplet; br, broad.

### Preparative HPLC Conditions

Preparative HPLC purification was performed by reverse phase HPLC using a Waters Fraction lynx preparative HPLC system (2525 pump, 2996/2998 UV/VIS detector, 2767 liquid handler) or an equivalent HPLC system such as a Gilson Trilution UV directed system. The Waters 2767 liquid handler acted as both auto-sampler and fraction collector. The columns used for the preparative purification of the compounds were a Waters Sunfire OBD Phenomenex Luna Phenyl Hexyl or Waters Xbridge Phenyl at 10 μm 19×150 mm or Waters CSH Phenyl Hexyl, 19×150, 5 μm column. Appropriate focused gradients were selected based on acetonitrile and methanol solvent systems under either acidic or basic conditions. The modifiers used under acidic/basic conditions were formic acid or trifluoroacetic acid (0.1% V/V) and ammonium bicarbonate (10 mM) respectively. The purification was controlled by Waters Fractionlynx software through monitoring at 210-400 nm and triggered a threshold collection value at 260 nm and, when using the Fractionlynx, the presence of target molecular ion as observed under API conditions. Collected fractions were analysed by LCMS (Waters Acquity systems with Waters SQD).

### Chiral Supercritical Fluid Chromatography (SFC) Separation Protocol

The separation of isomers was achieved by Supercritical Fluid Chromatography (SFC) using a Waters Thar Prep150 preparative SFC system (P200X CO₂ pump, 2545 modifier pump, 2998 UV/VIS detector, 2767 liquid handler with Stacked Injection Module, QDa Mass Spec). The Waters 2767 liquid handler acted as both auto-sampler and fraction collector. Appropriate isocratic methods were selected based on methanol, ethanol or isopropanol solvent systems under un-modified or basic conditions. The standard SFC method used was modifier, CO₂, 100 mL/min, 120 Bar backpressure, 40° C. column temperature. The modifier used under basic conditions was diethylamine (0.1% V/V). The modifier used under acidic conditions was either formic acid (0.1% V/V) or trifluoroacetic acid (0.1% V/V). The SFC purification was controlled by Waters Fractionlynx software through monitoring at 210-400 nm and triggered at a threshold collection value, typically 260 nm. Collected fractions were analysed by SFC (Waters/Thar SFC systems with Waters SQD or Waters UPCC with Waters QDa). The fractions that contained the desired product were concentrated by vacuum centrifugation.

### Supercritical Fluid Chromatography—Mass Spectrometry Analytical Conditions

Method 1: SFC-MS was performed on a Waters/Thar SFC systems with Waters SQD using a Lux Amylose-1 column with a 30% methyl alcohol/C₀₂ (with 0.1% ammonium hydroxide) isocratic run at 5 mL/min, 120 Bar backpressure, 40° C. column temperature.

Method 2: SFC-MS was performed on a Waters/Thar SFC systems with Waters SQD using a Lux Amylose-1 column with a 30% ethyl alcohol/C₀₂ (with 0.1% ammonium hydroxide) isocratic run at 5 mL/min, 120 Bar backpressure, 40° C. column temperature.

In the procedures that follow, after each starting material, reference to a compound number is sometimes provided. This is provided merely for assistance to the skilled chemist. The starting material may not necessarily have been prepared from the batch referred to.

When reference is made to the use of a “similar” or “analogous” procedure, as will be appreciated by those skilled in the art, such a procedure may involve minor variations, for example reaction temperature, reagent/solvent amount, reaction time, work-up conditions, or chromatographic purification conditions.

### Intermediate A1

### (S)-3-(4-bromo-2-fluorophenyl)-2-((tert-butoxycarbonyl)amino)propanoic acid

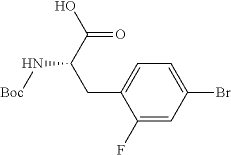 [SMILES: CC(C)(C)OC(=O)N[C@@H](Cc1ccc(Br)cc1F)C(=O)O]

A mixture of (2S)-2-amino-3-(4-bromo-2-fluoro-phenyl)propanoic acid (2.5 g, 9.54 mmol), di-tert-butyl dicarbonate (2.4 mL, 10.5 mmol) and triethylamine (2.7 mL, 19.1 mmol) in dichloromethane (40 mL) was stirred at r.t. for 2 days. An initial beige suspension formed, which later dissolved. The mixture was diluted with saturated aqueous NaHCO₃ (40 mL) and extracted with DCM (2×40 mL). The combined organic phases were washed with brine (30 mL), dried (MgSO₄), filtered and concentrated to provide crude material (4.29 g), which was used without further purification.

¹H NMR (400 MHz, CDCl₃) δ 7.18-7.11 (m, 3H), 5.43 (d, J=7.4 Hz, 1H), 4.36-4.25 (m, 1H), 3.31-3.19 (m, 1H), 1.39 (s, 9H). Two exchangeable protons not visible.

### Intermediate B1

### tert-butyl (S)-(1-amino-3-(4-bromo-2-fluorophenyl)-1-oxopropan-2-yl)carbamate

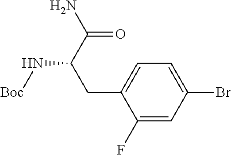 [SMILES: CC(C)(C)OC(=O)N[C@@H](Cc1ccc(Br)cc1F)C(N)=O]

A mixture of (S)-3-(4-bromo-2-fluorophenyl)-2-((tert-butoxycarbonyl)amino)propanoic acid (Intermediate A1, 3.45 g, 9.53 mmol), di-tert-butyl dicarbonate (3.3 mL, 14.3 mmol), ammonium acetate (1.10 g, 14.3 mmol) and pyridine (3.9 mL, 47.6 mmol) in 1,4-dioxane (45 mL) was stirred at r.t. for 16 hours. The reaction was concentrated, triturated with water and the resulting solid collected by filtration. The solid was dried in a vacuum oven at 40° C. for 2 hours to give crude material (4.86 g), which was used without further purification.

¹H NMR (400 MHz, CDCl₃) δ, 7.24 (d, J=8.3 Hz, 2H), 7.12 (dd, J=7.9, 7.9 Hz, 1H), 6.06 (s, 1H), 5.53-5.44 (m, 1H), 5.10-5.01 (m, 1H), 4.39 (d, J=6.4 Hz, 1H), 3.16 (dd, J=5.7, 14.0 Hz, 1H), 3.04-2.94 (m, 1H), 1.38 (s, 9H).

### Intermediate C1

### tert-butyl (S)-(1-amino-3-(4-iodophenyl)-1-oxopropan-2-yl)carbamate

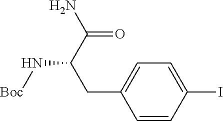 [SMILES: CC(C)(C)OC(=O)N[C@@H](Cc1ccc(I)cc1)C(N)=O]

A solution of (S)-2-((tert-butoxycarbonyl)amino)-3-(4-iodophenyl)propanoic acid (20.0 g, 51.1 mmol) in DMF (200 mL) at 0° C. was treated portion-wise with HATU (23.3 g, 61.3 mmol) and stirred cold for 15 minutes. Ammonium hydroxide solution (100 mL, 0.770 mol, 30%) was added dropwise over 15 minutes. After 3 hours at 0° C. the mixture was allowed to warm to ambient temperature and left to stand overnight. The mixture was diluted with water (100 mL) and extracted into EtOAc (2×50 mL). The combined extracts were washed with sat. aq. NaHCO₃ (50 mL), 1 M citric acid (50 mL), water (50 mL) and brine (50 mL), dried over MgSO₄, filtered and concentrated in vacuo to afford the title compound (20.12 g, quantitative yield) as a white solid.

¹H NMR (400 MHz, DMSO): δ 7.64 (d, J=8.2 Hz, 2H), 7.38 (s, 1H), 7.09 (d, J=8.3 Hz, 2H), 7.02 (s, 1H), 6.83 (d, J=8.8 Hz, 1H), 4.11-4.03 (m, 1H), 2.91-2.88 (m, 1H), 2.73-2.66 (m, 1H), 1.29 (s, 9H).

### Intermediate D1

### tert-butyl (S)-(1-cyano-2-(4-iodophenyl)ethyl)carbamate

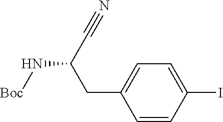 [SMILES: CC(C)(C)OC(=O)N[C@H](C#N)Cc1ccc(I)cc1]

A suspension of tert-butyl (S)-(1-amino-3-(4-iodophenyl)-1-oxopropan-2-yl)carbamate (Intermediate C1, 19.95 g, 51.1 mmol) and Et₃N (14 mL, 0.102 mol) in DCM (500 mL) at 0° C. was treated dropwise with trifluoroacetic anhydride (11 mL, 76.7 mmol). Once the solid had dissolved (˜10 minutes) the cooling bath was removed, and the pale-yellow solution stirred at ambient temperature for 1.5 hours. The mixture was diluted with DCM, washed with sat. NaHCO₃, water and brine, dried over MgSO₄ and concentrated in vacuo. The residue was purified by silica gel chromatography eluting with 0-15% EtOAc in DCM to afford tert-butyl (S)-(1-cyano-2-(4-iodophenyl)ethyl)carbamate (13.86 g, 73%) as an off-white solid.

¹H NMR (400 MHz, CDCl₃): δ 7.70 (d, J=8.4 Hz, 2H), 7.03 (d, J=8.3 Hz, 2H), 4.82-4.78 (m, 2H), 3.09-2.98 (m, 2H), 1.44 (s, 9H).

The Intermediates in the following Table 2 were prepared from reagents reported below following similar procedures as for Intermediate D1.

TABLE 2

| Int. | Structure & Name | Reagent | Analytical data |
|---|---|---|---|
| D2 | 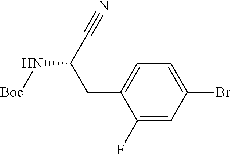 [SMILES: CC(C)(C)OC(=O)N[C@H](C#N)Cc1ccc(Br)cc1F] | B1 | ¹H NMR (400 MHz, CDCl3) δ 7.32- 7.27 (m, 2H), 7.17 (dd, J = 8.1, 8.1 Hz, 1H), 4.92-4.76 (m, 2H), 3.18- 3.06 (m, 2H), 1.43 (s, 9H). |
| | tert-butyl (S)-(2-(4-bromo-2- | | |
| | fluorophenyl)-1- | | |
| | cyanoethyl)carbamate | | |

### Intermediate E1

### tert-butyl (S)-(1-cyano-2-(4′-cyano-[1,1′-biphenyl]-4-yl)ethyl)carbamate

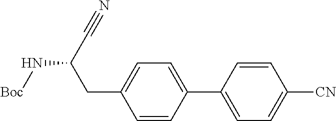 [SMILES: CC(C)(C)OC(=O)N[C@H](C#N)Cc1ccc(-c2ccc(C#N)cc2)cc1]

A mixture of tert-butyl (S)-(1-cyano-2-(4-iodophenyl)ethyl)carbamate (Intermediate D1, 8.14 g, 21.9 mmol), 4-cyanophenylboronic acid (3.61 g, 24.6 mmol), potassium carbonate (6.80 g, 49.2 mmol) and Pd(dppf)Cl₂ complex with dichloromethane (1.81 g, 2.19 mmol) in 1,4-dioxane (125 mL) and water (20 mL) was degassed, purged with nitrogen and heated at 80° C. for 2 hours. The cooled mixture was diluted with EtOAc, washed with water and brine, dried over MgSO₄ and concentrated in vacuo to leave an orange/brown solid. The residue was purified by silica gel chromatography eluting with 0-50% EtOAc in CycloHexane to the title compound (6.41 g, 18.5 mmol, 84%) as a pale orange solid.

¹H NMR (400 MHz, DMSO): δ 7.96-7.88 (m, 4H), 7.74 (d, J=8.4 Hz, 2H), 7.46 (d, J=8.3 Hz, 2H), 4.72 (q, J=7.9 Hz, 1H), 3.15-3.09 (m, 2H), 1.37 (s, 9H). Exchangeable amide proton not observed.

The Intermediates in the following Table 3 were prepared from reagents reported below following similar procedures as for Intermediate E1 using the suitable commercially available boronic acids.

TABLE 3

| Int. | Structure & Name | Reagent | Analytical data |
|---|---|---|---|
| E2 | 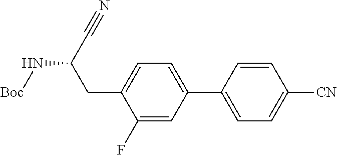 [SMILES: CC(C)(C)OC(=O)N[C@H](C#N)Cc1ccc(-c2ccc(C#N)cc2)cc1F] | D2 | ¹H NMR (400 MHz, CDCl₃) δ 7.75 (d, J = 8.4 Hz, 2 H), 7.66 (d, J = 8.5 Hz, 2 H), 7.42-7.32 (m, 4 H), 4.89 (s, 1 H), 3.21 (dd, J = 3.3, 6.3 Hz, 2 H), 1.54 (s, 9H). |
| | tert-butyl (S)-(1-cyano-2-(4′-cyano- | | |
| | 3-fluoro-[1,1′-biphenyl]-4- | | |
| | yl)ethyl)carbamate | | |
| E3 | 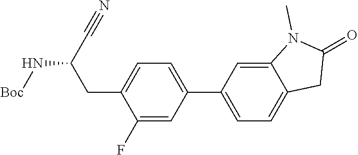 [SMILES: CN1C(=O)Cc2ccc(-c3ccc(C[C@@H](C#N)NC(=O)OC(C)(C)C)c(F)c3)cc21] | D2 | ¹H NMR (400 MHz, CDCl₃): δ 7.38-7.30 (m, 4 H), 7.23 (dd, J = 1.6, 7.7 Hz, 1 H) ,6.97 (d, J = 1.0 Hz, 1 H), 4.91 (d, J = 19.3 Hz, 2 H), 3.57 (s, 2 H), 3.27 (s, 3 H), 3.20 (dd, J = 3.6, 6.8 Hz, 2 H), 1.44 (s, 9 H). |
| | tert-butyl (S)-(1-cyano-2-(2-fluoro- | | |
| | 4-(1-methyl-2-oxoindolin-6- | | |
| | yl)phenyl)ethyl)carbamate | | |

### Intermediate F1

### (S)-4′-(2-amino-2-cyanoethyl)-[1,1′-biphenyl]-4-carbonitrile

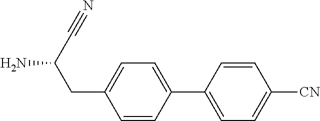 [SMILES: N#Cc1ccc(-c2ccc(C[C@H](N)C#N)cc2)cc1]

Tert-butyl (S)-(1-cyano-2-(4′-cyano-[1,1′-biphenyl]-4-yl)ethyl)carbamate (Intermediate E1, 5.34 g, 15.4 mmol) was suspended in formic acid (30 mL) at r.t. The mixture was stirred in a pre-heated block at 50° C. for 45 min. The formic acid was removed by evaporation and the residue diluted with 100 mL EtOAc. The solution was washed with saturated aqueous Na₂CO₃ (80 mL) and the layers were separated. The aqueous layer was extracted with EtOAc (50 mL); the combined organics were dried (MgSO₄), filtered and concentrated in vacuo. Purification by silica gel chromatography, eluting with 20-100% EtOAc in cyclohexane, gave the title compound (2.77 g, 11.2 mmol, 73%).

¹H NMR (400 MHz, CDCl₃) δ 7.75-7.72 (m, 2H), 7.69-7.66 (m, 2H), 7.59 (d, J=8.3 Hz, 2H), 7.43 (d, J=8.4 Hz, 2H), 3.99 (s, 1H), 3.12-3.08 (m, 2H), 1.65 (br s, 2H).

The Intermediates in the following Table 4 were prepared from reagents reported below following similar procedures as for Intermediate F1.

TABLE 4

| Int. | Structure & Name | Reagent | Analytical data |
|---|---|---|---|
| F2 | 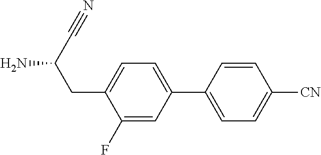 [SMILES: N#Cc1ccc(-c2ccc(C[C@H](N)C#N)c(F)c2)cc1] | E2 | ¹H NMR (400 MHz, CDCl₃) δ 7.75 (d, J = 8.5 Hz, 2 H), 7.66 (d, J = 8.5 Hz, 2 H), 7.45-7.36 (m, 2 H), 7.32 (dd, J = 1.7, 10.7 Hz, 1 H), 4.15-3.99 (m, 1 H), 3.15 (dd, J = 7.6, 7.6 Hz, 2 H). NH₂ protons not observed. |
| | (S)-4′-(2-amino-2- | | |
| | cyanoethyl)-3′-fluoro-[1,1′- | | |
| | biphenyl]-4-carbonitrile | | |
| F3 | 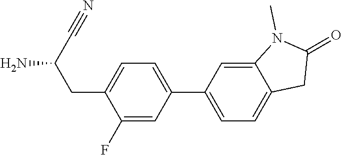 [SMILES: CN1C(=O)Cc2ccc(-c3ccc(C[C@H](N)C#N)c(F)c3)cc21] | E3 | ¹H NMR (400 MHz, CDCl₃): δ 7.42-7.29 (m, 4 H), 7.23 (dd, J = 1.6, 7.6 Hz, 1 H), 6.97 (d, J = 1.2 Hz, 1 H), 4.04 (dd, J = 7.1, 7.1 Hz, 1 H), 3.57 (s, 2 H), 3.27 (s, 3 H), 3.18-3.11 (m, 2 H). NH₂ protons not observed. |
| | (S)-2-amino-3-(2-fluoro-4- | | |
| | (1-methyl-2-oxoindolin-6- | | |
| | yl)phenyl)propanenitrile | | |
| F4 | 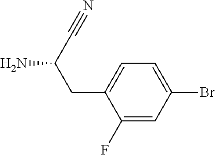 [SMILES: N#C[C@@H](N)Cc1ccc(Br)cc1F] | D2 | ¹H NMR (400 MHz, CDCl₃): δ 7.35-7.25 (m, 2 H), 7.22-7.16 (m, 1 H), 3.95 (t, J = 7.1 Hz, 1 H), 3.10-2.99 (m, 2 H). NH₂ protons not observed. |
| | (S)-2-amino-3-(4-bromo-2- | | |
| | fluorophenyl)propanenitrile | | |
| F5 | 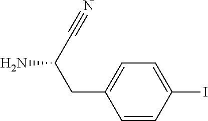 [SMILES: N#C[C@@H](N)Cc1ccc(I)cc1] | D1 | ¹H NMR (400 MHz, CDCl₃): δ 7.69 (d, J = 8.3 Hz, 2 H), 7.05 (d, J = 8.3 Hz, 2 H), 3.91 (dd, J = 6.4, 6.4 Hz, 1 H), 2.97 (dd, J = 3.4, 6.4 Hz, 2 H), 1.84 (s, 2 H). |
| | (S)-2-amino-3-(4- | | |
| | iodophenyl)propanenitrile | | |

### Intermediate G1

### 2-(Benzhydrylideneamino)-3-(5-bromo-3-fluoro-2-pyridyl)propanenitrile

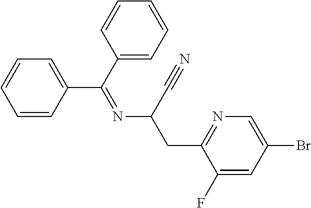 [SMILES: N#CC(Cc1ncc(Br)cc1F)N=C(c1ccccc1)c1ccccc1]

To a solution of 5-bromo-2-(chloromethyl)-3-fluoro-pyri dine (500 mg, 2.23 mmol) in dichloromethane (10 mL) was successively added N-(diphenylmethylene)aminoacetonitrile (500 mg, 2.27 mmol, 1.02 eq), benzyltriethylammonium chloride (56 mg, 0.246 mmol, 0.110 eq), sodium hydroxide (190 mg, 4.75 mmol, 2.13 eq) and water (1 mL). The mixture was stirred vigorously at room temperature for 16 hours. Reaction was diluted with H₂O (10 mL) and the organic phase was collected. The aqueous phase was extracted with dichloromethane (2×10 mL). The combined organic phases were filtered through a phase separator frit and the solvent was removed in vacuo. The residue was purified by column chromatography on silica gel, eluting with 0-40% ethyl acetate in cyclohexane to give the title compound (660 mg, 1.62 mmol, 73%).

LCMS (method BicarbBEHC18): [MH+]=408 at 5.80 min.

### Intermediate H1

### 4-[6-[2-(benzhydrylideneamino)-2-cyano-ethyl]-5-fluoro-3-pyridyl]benzonitrile

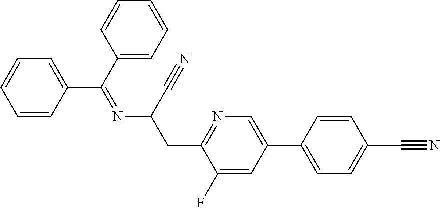 [SMILES: N#Cc1ccc(-c2cnc(CC(C#N)N=C(c3ccccc3)c3ccccc3)c(F)c2)cc1]

To a solution of 2-(benzhydrylideneamino)-3-(5-bromo-3-fluoro-2-pyridyl)propanenitrile (Intermediate G1, 660 mg, 1.62 mmol) in acetonitrile (8 mL) and water (2 mL) in a screw-cap reaction tube, was successively added 4-cyanophenylboronic acid (261 mg, 1.78 mmol, 1.10 eq) and potassium phosphate tribasic monohydrate (745 mg, 3.23 mmol). The mixture was degassed with nitrogen for 5 min then [1,1′-bis(diphenylphosphino)ferrocene]dichloropalladium(II) complex with dichloromethane (134 mg, 0.162 mmol) was added. The resulting mixture was stirred at 80° C. for 3 hours. After return to room temperature, reaction was diluted with ethyl acetate (50 mL) and washed with saturated aqueous solution of NaHCO₃ (2×10 mL). The organic phase was collected, filtered through a phase separator frit. The solvent was removed in vacuo and the residue was purified by column chromatography on silica gel, eluting with 0-50% ethyl acetate in cyclohexane to give the title compound (640 mg, 1.49 mmol, 92%).

LCMS (method BicarbBEHC18): [MH+]=431 at 5.66 min.

### Intermediate I1

### 4-[6-(2-amino-2-cyano-ethyl)-5-fluoro-3-pyridyl]benzonitrile

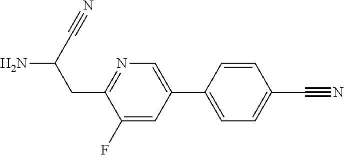 [SMILES: N#Cc1ccc(-c2cnc(CC(N)C#N)c(F)c2)cc1]

To a solution of 4-[6-[2-(benzhydrylideneamino)-2-cyano-ethyl]-5-fluoro-3-pyridyl]benzonitrile (Intermediate H1, 660 mg, 1.53 mmol) in tetrahydrofuran (6 mL) was added 2N aqueous solution of hydrogen chloride (1.5 mL). The resulting mixture was stirred for three hours at room temperature. Reaction was diluted with diethyl ether (50 mL) and water (10 mL). The phases were separated, and the aqueous phase was basified with 1N NaOH. The aqueous layer was extracted with dichloromethane (3×10 mL). Combined DCM phases were filtered through a phase separator frit and the solvent was removed in vacuo to give the title compound (400 mg, 1.41 mmol, 92%).

¹H NMR (400 MHz, DMSO): δ 8.86 (s, 1H), 8.20 (dd, J=1.9, 10.9 Hz, 1H), 8.01 (q, J=8.7 Hz, 4H), 4.24 (dd, J=7.5, 7.5 Hz, 1H), 3.30-3.16 (m, 2H), 2.70 (brs, 2H). LCMS (method AcHSSC18): [MH+]=267.2 at 2.79 min.

### Example 1

### (S)—N-(1-cyano-2-(4′-cyano-[1,1′-biphenyl]-4-yl)ethyl)-3-hydroxyazetidine-3-carboxamide

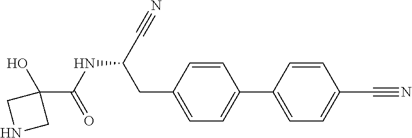 [SMILES: N#Cc1ccc(-c2ccc(C[C@@H](C#N)NC(=O)C3(O)CNC3)cc2)cc1]

A mixture of 1-tert-butoxycarbonyl-3-hydroxy-azetidine-3-carboxylic acid (44 mg, 0.20 mmol), N-(3-dimethylaminopropyl)-N-ethylcarbodiimide hydrochloride (46.5 mg, 0.24 mmol) and 2-pyridinol 1-oxide (27 mg, 0.24 mmol) in dichloromethane (2 mL) was stirred at room temperature for 30 min, then (S)-4′-(2-amino-2-cyanoethyl)-[1,1′-biphenyl]-4-carbonitrile (Intermediate F1, 50 mg, 0.20 mmol) was added followed by N,N-diisopropylethylamine (0.052 mL, 0.3 mmol). The mixture was stirred at room temperature for 16 hours. The mixture was diluted with dichloromethane (15 mL) and 2 N HCl (3 mL). The aqueous layer was extracted with dichloromethane (2×15 mL). The organic layers were combined, washed with saturated aqueous NaHCO₃, dried over MgSO₄, filtered, and concentrated in vacuo. The isolated material was stirred in formic acid (0.8 mL) at 50° C. for 10 min then allowed to cool to room temperature. The formic acid was removed in vacuo. The mixture was diluted with dichloromethane (15 mL) and shaken with saturated aqueous NaHCO₃ (5 mL). The organic layer was separated, and the aqueous layer was extracted with dichloromethane (2×15 mL). The combined organic phases were dried over MgSO₄, filtered and concentrated in vacuo. The crude material was purified by reverse phase HPLC to give the title compound (4.7 mg, 13 μmol, 6%) as an off-white solid.

¹H NMR (400 MHz, DMSO): δ 8.61 (br s, 1H), 7.91 (d, J=8.8 Hz, 2H), 7.87 (d, J=8.4 Hz, 2H), 7.71 (d, J=8.2 Hz, 2H), 7.42 (d, J=8.2 Hz, 2H), 6.49 (br s, 1H), 5.04 (s, 1H), 3.74 (d, J=8.7 Hz, 1H), 3.49 (d, J=7.0 Hz, 1H), 3.23 (d, J=6.9 Hz, 2H). 2 protons obscured by water peak. One exchangeable proton not observed. LCMS (Method AcHSSC18): [MH+]=347.2 at 3.06 min.

The following compounds were prepared following similar procedures as for Example 1 from commercially available Boc-azetidine-carboxylic acids, and were reported below in Table 5.

TABLE 5

| Ex. | Structure & Name | Reagent | Analytical data |
|---|---|---|---|
| 2 | 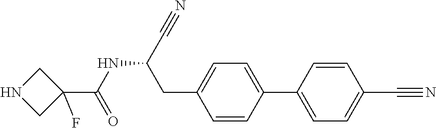 [SMILES: N#Cc1ccc(-c2ccc(C[C@@H](C#N)NC(=O)C3(F)CNC3)cc2)cc1] | F1 | ¹H NMR (400 MHz, DMSO): δ 9.11 (d, J = 8.3 Hz, 1 H), 7.92 (d, J = 8.7 Hz, 2 H), 7.88 (d, J = 8.4 Hz, 2 H), 7.72 (d, J = 8.3 Hz, 2 H), 7.42 (d, J = 8.3 Hz, 2 H), 5.09 (q, J = 7.8 Hz, 1 H), 3.91-3.81 (m, 1 H). exchangeable amine proton not observed. 5 protons obscured by water signal. LCMS |
| | (S)-N-(1-cyano-2-(4′-cyano- | | (Method BicarbBEHC18): [MH+] = |
| | [1,1′-biphenyl]-4-yl)ethyl)-3- | | 349.2 at 3.63 min. |
| | fluoroazetidine-3-carboxamide | | |
| 3 | 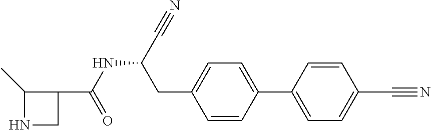 [SMILES: CC1NCC1C(=O)N[C@H](C#N)Cc1ccc(-c2ccc(C#N)cc2)cc1] | F1 | ¹H NMR (400 MHz, DMSO): δ 8.62 (d, J = 7.8 Hz, 1 H), 7.94-7.86 (m, 4 H), 7.72 (dd, J = 2.5, 8.3 Hz, 2 H), 7.42 (d, J = 8.1 Hz, 2 H), 5.06-4.94 (m, 1 H), 3.88-3.61 (m, 1 H), 3.51- 3.41 (m, 1 H), 3.40-3.34 (m, 1 H), 3.20-3.09 (m, 3 H), 2.87-2.78 (m, 1 H), 1.07 (dd, J = 6.2, 20.3 Hz, 3 H). |
| | N-((S)-1-cyano-2-(4′-cyano- | | LCMS (method AcHSSC18): [MH+] = |
| | [1,1′-biphenyl]-4-yl)ethyl)-2- | | 345 at 3.15 min. |
| | methylazetidine-3-carboxamide | | |
| 4 | 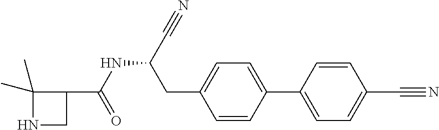 [SMILES: CC1(C)NCC1C(=O)N[C@H](C#N)Cc1ccc(-c2ccc(C#N)cc2)cc1] | F1 | ¹H NMR (400 MHz, DMSO): δ 8.76 (d, J = 7.1 Hz, 1 H), 8.32 (s, 1 H), 7.94-7.83 (m, 4 H), 7.72 (dd, J = 8.3, 11.1 Hz, 2 H), 7.46 (dd, J = 4.9, 8.2 Hz, 2 H), 5.11-5.02 (m, 1 H), 3.74- 3.67 (m, 2 H), 3.45-3.33 (m, 1 H), 3.28-3.04 (m, 6 H), 1.42-0.70 (m, 6 H). |
| | N-((S)-1-cyano-2-(4′-cyano- | | LCMS (method AcHSSC18): [MH+] = |
| | [1,1′-biphenyl]-4-yl)ethyl)-2,2- | | 359 at 3.19 min. |
| | dimethylazetidine-3- | | |
| | carboxamide | | |
| 5 | 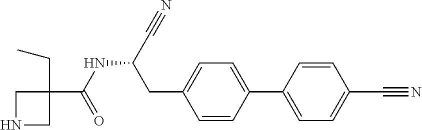 [SMILES: CCC1(C(=O)N[C@H](C#N)Cc2ccc(-c3ccc(C#N)cc3)cc2)CNC1] | F1 | ¹H NMR (400 MHz, DMSO): δ 8.60 (d, J = 8.2 Hz, 1 H), 8.31 (s, 1 H), 7.94-7.84 (m, 4 H), 7.72 (d, J = 8.2 Hz, 2 H), 7.45 (d, J = 8.2 Hz, 2 H), 5.10-5.03 (m, 1 H), 3.71 (d, J = 8.8 Hz, 2 H), 3.30-3.10 (m, 4 H), 1.86- 1.71 (m, 2 H), 0.50 (dd, J = 7.3, 7.3 Hz, 3 H). |
| | (S)-N-(1-cyano-2-(4′-cyano- | | LCMS (method BicarbBEHC18): |
| | [1,1′-biphenyl]-4-yl)ethyl)-3- | | [MH+] = 359 at 3.68 min. |
| | ethylazetidine-3-carboxamide | | |
| 6 | 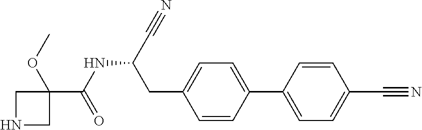 [SMILES: COC1(C(=O)N[C@H](C#N)Cc2ccc(-c3ccc(C#N)cc3)cc2)CNC1] | F1 | ¹H NMR (400 MHz, DMSO): δ 8.82 (d, J = 8.2 Hz, 1 H), 7.93-7.85 (m, 4 H), 7.71 (d, J = 8.3 Hz, 2 H), 7.44 (d, J = 8.3 Hz, 2 H), 5.12-5.05 (m, 1 H), 3.67 (d, J = 9.3 Hz, 1 H), 3.50-3.40 (m, 3 H), 3.27-3.17 (m, 3 H), 3.01 (s, 3 H). LCMS (method BicarbBEHC18): |
| | (S)-N-(1-cyano-2-(4′-cyano- | | [MH+] = 381 at 3.71 min |
| | [1,1′-biphenyl]-4-yl)ethyl)-3- | | |
| | methoxyazetidine-3- | | |
| | carboxamide | | |
| 7 | 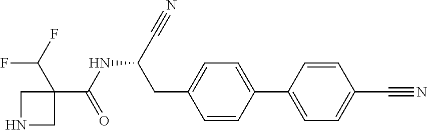 [SMILES: N#Cc1ccc(-c2ccc(C[C@@H](C#N)NC(=O)C3(C(F)F)CNC3)cc2)cc1] | F1 | ¹H NMR (400 MHz, DMSO): δ 8.92 (d, J = 7.3 Hz, 1 H), 7.94-7.87 (m, 4 H), 7.73 (d, J = 8.3 Hz, 2 H), 7.45 (d, J = 8.3 Hz, 2 H), 6.25 (t, J = 56.8 Hz, 1 H), 5.08 (q, J = 7.6 Hz, 1 H), 3.62- 3.54 (m, 4 H), 3.25-3.15 (m, 3 H). LCMS (method BicarbBEHC18): [MH+] = 381 at 3.71 min. |
| | (S)-N-(1-cyano-2-(4′-cyano- | | |
| | [1,1′-biphenyl]-4-yl)ethyl)-3- | | |
| | (difluoromethyl)azetidine-3- | | |
| | carboxamide | | |
| 8 | 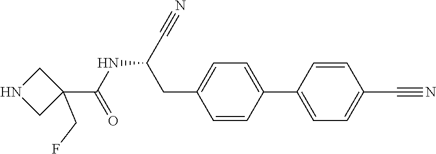 [SMILES: N#Cc1ccc(-c2ccc(C[C@@H](C#N)NC(=O)C3(CF)CNC3)cc2)cc1] | F1 | ¹H NMR (400 MHz, DMSO): δ 8.81 (d, J = 7.8 Hz, 1 H), 7.92 (d, J = 8.8 Hz, 2 H), 7.88 (d, J = 8.5 Hz, 2 H), 7.73 (d, J = 8.2 Hz, 2 H), 7.45 (d, J = 8.2 Hz, 2 H), 5.06 (q, J = 7.3 Hz, 1 H), 4.69 (dq, J = 9.4, 20.8 Hz, 2 H), 3.67 (ddd, J = 2.6, 8.8, 15.3 Hz, 2 H), 3.35 (dd, J = 6.3, 8.7 Hz, 2 H), 3.24- 3.13 (m, 2 H). 1 exchangeable proton not observed. |
| | (S)-N-(1-cyano-2-(4′-cyano- | | LCMS (Method AcHSSC18): [MH−] = |
| | [1,1′-biphenyl]-4-yl)ethyl)-3- | | 361.2 at 3.2 min. |
| | (fluoromethyl)azetidine-3- | | |
| | carboxamide | | |
| 9 | 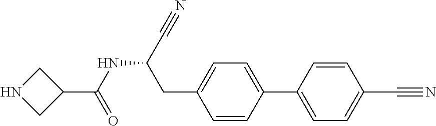 [SMILES: N#Cc1ccc(-c2ccc(C[C@@H](C#N)NC(=O)C3CNC3)cc2)cc1] | F1 | ¹H NMR (400 MHz, DMSO): δ 8.67 (dd, J = 7.5, 7.5 Hz, 1 H), 7.94-7.87 (m, 4 H), 7.73 (d, J = 8.3 Hz, 2 H), 7.43 (d, J = 8.3 Hz, 2 H), 5.03-4.97 (m, 1 H), 3.64-3.59 (m, 1 H), 3.52- 3.39 (m, 4 H), 3.20-3.07 (m, 3 H). LCMS (method BicarbBEHC18): [MH+] = 331 at 3.44 min. |
| | (S)-N-(1-cyano-2-(4′-cyano- | | |
| | [1,1′-biphenyl]-4- | | |
| | yl)ethyl)azetidine-3- | | |
| | carboxamide | | |
| 10 | 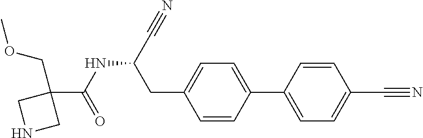 [SMILES: COCC1(C(=O)N[C@H](C#N)Cc2ccc(-c3ccc(C#N)cc3)cc2)CNC1] | F1 | ¹NMR (400 MHz, DMSO): δ 8.49- 8.44 (m, 1 H), 7.94-7.86 (m, 4 H), 7.73 (d, J = 8.3 Hz, 2 H), 7.44 (d, J = 8.3 Hz, 2 H), 5.06-4.98 (m, 1 H), 3.65-3.51 (m, 4 H), 3.25-3.17 (m, 8 H). LCMS (method BicarbBEHC18): [MH+] = 375 at 3.60 min. |
| | (S)-N-(1-cyano-2-(4′-cyano- | | |
| | [1,1′-biphenyl]-4-yl)ethyl)-3- | | |
| | (methoxymethyl)azetidine-3- | | |
| | carboxamide | | |
| 11 | 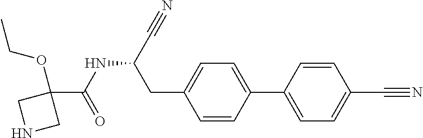 [SMILES: CCOC1(C(=O)N[C@H](C#N)Cc2ccc(-c3ccc(C#N)cc3)cc2)CNC1] | F1 | ¹H NMR (400 MHz, DMSO): δ 8.65 (d, J = 8.3 Hz, 1 H), 7.93-7.85 (m, 4 H), 7.71 (d, J = 8.3 Hz, 2 H), 7.43 (d, J = 8.3 Hz, 2 H), 5.14-5.06 (m, 1 H), 3.58 (d, J = 8.8 Hz, 1 H), 3.48 (d, J = 8.8 Hz, 1 H), 3.42-3.38 (m, 1 H), 3.37-3.19 (m, 4 H), 3.16-3.08 (m, 1 H), 3.00-2.92 (m, 1 H), 1.05 (dd, J = 6.9, 6.9 Hz, 3 H). |
| | (S)-N-(1-cyano-2-(4′-cyano- | | LCMS (method BicarbBEHC18): |
| | [1,1′-biphenyl]-4-yl)ethyl)-3- | | [MH+] = 375 at 4.0 min. |
| | ethoxyazetidine-3-carboxamide | | |
| 12 | 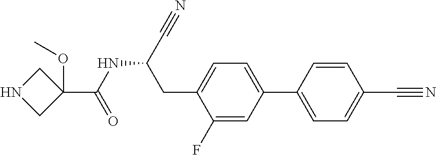 [SMILES: COC1(C(=O)N[C@H](C#N)Cc2ccc(-c3ccc(C#N)cc3)cc2F)CNC1] | F2 | ¹H NMR (400 MHz, DMSO) d 8.90 (d, J = 8.3 Hz, 1 H), 7.98 (s, 4 H), 7.72 (dd, J = 1.6, 11.2 Hz, 1 H), 7.66 (dd, J = 1.8, 8.1 Hz, 1H), 7.56 (dd, J = 7.8, 7.8 Hz, 1 H), 5.20-5.12 (m, 1 H), 3.68 (d, J = 8.8 Hz, 1 H), 3.55- 3.43 (m, 4 H), 3.34-3.27 (m, 2 H), 3.12 (s, 3 H). LCMS (Method AcHSSC18): [MH+] = 379.5 at 3.24 min. |
| | (S)-N-(1-cyano-2-(4′-cyano-3- | | LCMS (Method BicarbBEHC18): |
| | fluoro-[1,1′-biphenyl]-4- | | [MH+] = 379.5 at 3.87 min. |
| | yl)ethyl)-3-methoxyazetidine-3- | | |
| | carboxamide | | |
| 13 | 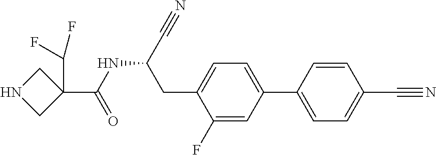 [SMILES: N#Cc1ccc(-c2ccc(C[C@@H](C#N)NC(=O)C3(C(F)F)CNC3)c(F)c2)cc1] | F2 | ¹H NMR (400 MHz, DMSO) d 9.05- 9.04 (m, 1 H), 7.99 (s, 4 H), 7.74 (d, J = 11.5 Hz, 1 H), 7.67 (dd, J = 1.8, 8.1 Hz, 1 H), 7.58 (dd, J = 8.0, 8.0 Hz, 1 H), 6.32 (t, J = 55.0 Hz, 1 H), 5.18 (d, J = 5.1 Hz, 1 H), 3.66 (d, J = 8.1 Hz, 1 H), 3.61 (s, 3 H), 3.34-3.22 (m, 2 H), 2.13 (s, 1 H). LCMS (Method AcHSSC18): [MH+] = 399.5 at 3.32 min. |
| | (S)-N-(1-cyano-2-(4′-cyano-3- | | LCMS (Method BicarbBEHC18): |
| | fluoro-[1,1′-biphenyl]-4- | | [MH+] = 399.5 at 3.97 min. |
| | yl)ethyl)-3- | | |
| | (difluoromethyl)azetidine-3- | | |
| | carboxamide | | |
| 16 | 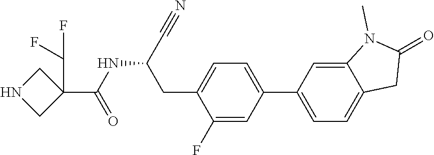 [SMILES: CN1C(=O)Cc2ccc(-c3ccc(C[C@@H](C#N)NC(=O)C4(C(F)F)CNC4)c(F)c3)cc21] | F3 | ¹H NMR (400 MHz, DMSO): δ 9.04 (d, J = 7.8 Hz, 1 H), 7.68-7.61 (m, 2 H), 7.54 (dd, J = 8.0, 8.0 Hz, 1 H), 7.41-7.37 (m, 3 H), 6.33 (t, J = 54.2 Hz, 1 H), 5.20-5.13 (m, 1 H), 3.69 (d, J = 8.6 Hz, 1 H), 3.65-3.60 (m, 5 H), 3.31-3.22 (m, 6 H). LCMS (Method AcHSSC18): [MH+] = 443.5 at 3.01 min. |
| | (S)-N-(1-cyano-2-(2-fluoro-4-(1- | | |
| | methyl-2-oxoindolin-6- | | |
| | yl)phenyl)ethyl)-3- | | |
| | (difluoromethyl)azetidine-3- | | |
| | carboxamide | | |
| 48 | 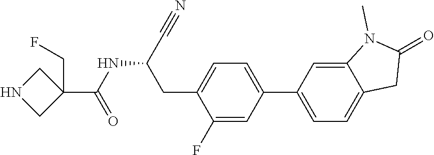 [SMILES: CN1C(=O)Cc2ccc(-c3ccc(C[C@@H](C#N)NC(=O)C4(CF)CNC4)c(F)c3)cc21] | F3 | ¹H NMR (400 MHz, DMSO): δ 8.80 (d, J = 8.1 Hz, 1H), 7.63-7.55 (m, 2H), 7.48 (dd, J = 8.3, 8.3 Hz, 1H), 7.37-7.32 (m, 3H), 5.09 (d, J = 6.6 Hz, 1H), 4.71 (dd, J = 4.7, 48.4 Hz, 2H), 3.63 (dd, J = 2.6, 8.4 Hz, 1H), 3.58 (s, 3H), 3.28-3.17 (m, 8H). 19F NMR (376 MHz, DMSO) δ −117.25 (s). LCMS (Method BicarbBEHC18): [MH+] = 425.2 at |
| | (S)-N-(1-cyano-2-(2-fluoro-4-(1- | | 3.46 min. |
| | methyl-2-oxoindolin-6- | | |
| | yl)phenyl)ethyl)-3- | | |
| | (fluoromethyl)azetidine-3- | | |
| | carboxamide | | |
| 49 | 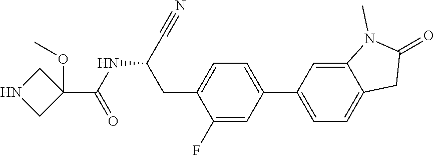 [SMILES: COC1(C(=O)N[C@H](C#N)Cc2ccc(-c3ccc4c(c3)N(C)C(=O)C4)cc2F)CNC1] | F3 | ¹H NMR (400 MHz, DMSO): δ 8.84 (d, J = 8.3 Hz, 1 H), 7.58-7.53 (m, 2 H), 7.46 (dd, J = 8.1, 8.1 Hz, 1 H), 7.32 (d, J = 9.3 Hz, 3 H), 5.12-5.05 (m, 1 H), 3.64 (d, J = 8.8 Hz, 1 H), 3.58 (s, 2 H), 3.52-3.36 (m, 3 H), 3.28-3.22 (m, 2 H), 3.19 (s, 3 H), 3.07 (s, 3 H). NH not observed. LCMS (Method AcHSSC18): [MH+] = 423.5 at 2.94 min. |
| | (S)-N-(1-cyano-2-(2-fluoro-4-(1- | | |
| | methyl-2-oxoindolin-6- | | |
| | yl)phenyl)ethyl)-3- | | |
| | methoxyazetidine-3- | | |
| | carboxamide | | |
| 31 | 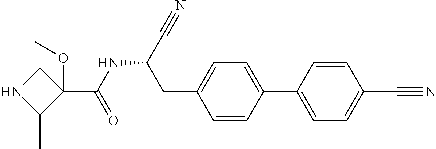 [SMILES: COC1(C(=O)N[C@H](C#N)Cc2ccc(-c3ccc(C#N)cc3)cc2)CNC1C] | F1 | ¹H NMR (400 MHz, DMSO): δ 8.79- 8.62 (m, 1 H), 7.93-7.82 (m, 4 H), 7.74-7.67 (m, 2 H), 7.49-7.39 (m, 2 H), 5.18-5.02 (m, 1 H), 3.78- 3.58 (m, 2 H), 3.37-3.19 (m, 3 H), 3.06 & 3.03 & 2.93 & 2.78 (4 × s, 3 H total), 1.09-0.52 (m, 3 H). One NH not observed. LCMS (Method AcHSSC18): [MH+] = 375.2 at 3.2 min. |
| | N-((S)-1-cyano-2-(4′-cyano- | | |
| | [1,1′-biphenyl]-4-yl)ethyl)-3- | | |
| | methoxy-2-methylazetidine-3- | | |
| | carboxamide | | |

### Intermediate J1

### tert-butyl 3-((1-cyano-2-(5-(4-cyanophenyl)-3-fluoropyridin-2-yl)ethyl)carbamoyl)-3-methoxyazetidine-1-carboxylate

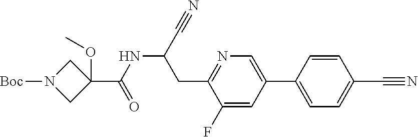 [SMILES: COC1(C(=O)NC(C#N)Cc2ncc(-c3ccc(C#N)cc3)cc2F)CN(C(=O)OC(C)(C)C)C1]

A mixture of 1-(tert-butoxycarbonyl)-3-methoxyazetidine-3-carboxylic acid (300 mg, 1.29 mmol), N-(3-dimethylaminopropyl)-N′-ethylcarbodiimide hydrochloride (320 mg, 1.67 mmol) and 2-pyridinol 1-oxide (175 mg, 1.57 mmol) in dichloromethane (10 mL) was stirred at room temperature for 30 min, then 4-[6-(2-amino-2-cyano-ethyl)-5-fluoro-3-pyridyl]benzonitrile (Intermediate I1, 350 mg, 1.31 mmol) was added followed by N,N-diisopropylethylamine (0.4 mL, 2.30 mmol). The mixture was stirred at room temperature for 16 hours. The mixture was diluted with dichloromethane (15 mL) and 2 N HCl (3 mL). The aqueous layer was extracted with dichloromethane (2×15 mL). The organic layers were combined, washed with saturated aqueous NaHCO₃, dried over MgSO₄, filtered, and concentrated in vacuo to give the title compound (490 mg, 78%) as an off-white solid.

LCMS (Method AcHSSC18): [MH+]=480.2 at 4.87 min.

### Example 42

### N-(1-cyano-2-(5-(4-cyanophenyl)-3-fluoropyridin-2-yl)ethyl)-3-methoxyazetidine-3-carboxamide

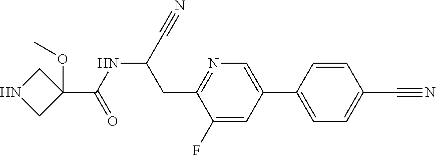 [SMILES: COC1(C(=O)NC(C#N)Cc2ncc(-c3ccc(C#N)cc3)cc2F)CNC1]

To a solution of tert-butyl 3-((1-cyano-2-(5-(4-cyanophenyl)-3-fluoropyridin-2-yl)ethyl)carbamoyl)-3-methoxyazetidine-1-carboxylate (Intermediate J1, 90 mg, 0.188 mmol, 1.00 eq) in dichloromethane (5 mL) at 0° C. was successively added trimethylsilyl trifluoromethanesulfonate (51 μL, 0.282 mmol) and 2,6-Lutidine (44 μL, 0.375 mmol). The mixture was stirred at 0° C. for 30 min then at room temperature for 2 hours. Reaction was quenched with saturated aqueous solution of ammonium chloride (10 mL). The organic phase was collected. The aqueous phase was extracted with dichloromethane (2×10 mL). Combined organic phases were filtered through a phase separator frit and the solvent was removed in vacuo. The crude material was purified by reverse phase HPLC to give the title compound (6.5 mg, 17 μmol, 9%) as an off-white solid.

¹H NMR (400 MHz, DMSO): δ 8.91-8.87 (m, 2H), 8.22 (dd, J=1.9, 11.0 Hz, 1H), 8.05-7.97 (m, 4H), 5.41-5.34 (m, 1H), 3.66 (d, J=9.0 Hz, 1H), 3.61-3.52 (m, 2H), 3.47-3.37 (m, 3H), 3.11 (s, 3H). LCMS (method AcHSSC18): [MH+]=380.4 at 2.89 min.

### Example 17

### (S)—N-(1-cyano-2-(4′-cyano-[1,1′-biphenyl]-4-yl)ethyl)-3-(trifluoromethyl)azetidine-3-carboxamide

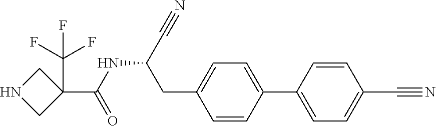 [SMILES: N#Cc1ccc(-c2ccc(C[C@@H](C#N)NC(=O)C3(C(F)(F)F)CNC3)cc2)cc1]

To a solution of 3-(trifluoromethyl)azetidine-3-carboxylic acid hydrochloride (105 mg, 0.511 mmol) in methanol (2 mL) was added triethylamine (214 μL, 1.53 mmol) and di-tert-butyl dicarbonate (0.15 mL, 0.641 mmol). The resulting mixture was stirred at room temperature for 16 hours. The solvent was removed in vacuo. The residue was taken up with dichloromethane (2 mL) and N-(3-dimethylaminopropyl)-N′-ethylcarbodiimide hydrochloride (115 mg, 0.60 mmol) and 2-pyridinol 1-oxide (65 mg, 0.58 mmol) were added. The resulting mixture was stirred at room temperature for 30 min, then (S)-4′-(2-amino-2-cyanoethyl)-[1,1′-biphenyl]-4-carbonitrile (Intermediate F1, 120 mg, 0.48 mmol) was added followed by N,N-diisopropylethylamine (0.15 mL, 0.86 mmol). The mixture was stirred at room temperature for 16 hours. The mixture was diluted with dichloromethane (15 mL) and 2 N HCl (3 mL). The aqueous layer was extracted with dichloromethane (2×15 mL). The organic layers were combined, washed with saturated aqueous NaHCO₃, dried over MgSO₄, filtered, and concentrated in vacuo. The isolated material was stirred in formic acid (1 mL) at 50° C. for 10 min then allowed to cool to room temperature. The formic acid was removed in vacuo. The mixture was diluted with dichloromethane (15 mL) and shaken with saturated aqueous NaHCO₃ (5 mL). The organic layer was separated, and the aqueous layer was extracted with dichloromethane (2×15 mL). The combined organic phases were dried over MgSO₄, filtered and concentrated in vacuo. The crude material was purified by reverse phase HPLC to give the title compound (12.0 mg, 29 μmol, 6%) as an off-white solid.

¹H NMR (400 MHz, DMSO): δ 9.13 (d, J=7.6 Hz, 1H), 7.94-7.86 (m, 4H), 7.73 (d, J=8.3 Hz, 2H), 7.45 (d, J=8.3 Hz, 2H), 5.14-5.07 (m, 1H), 3.78-3.60 (m, 4H), 3.24-3.18 (m, 2H), 2.69-2.66 (m, 1H). LCMS (Method AcHSSC18): [MH+]=399.2 at 3.39 min.

The following compounds were prepared from commercially available azetidine-carboxylic acids and reagents reported below in Table 6 following similar procedures as for Example 17.

TABLE 6

| Exam- | | | |
|---|---|---|---|
| ples | Structure & Name | Reagent | Analytical data |
| 18 | 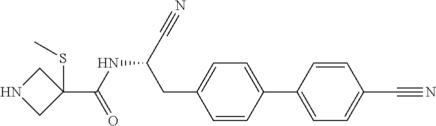 [SMILES: CSC1(C(=O)N[C@H](C#N)Cc2ccc(-c3ccc(C#N)cc3)cc2)CNC1] | F1 | ¹H NMR (400 MHz, DMSO): δ 8.72 (d, J = 8.1 Hz, 1 H), 7.94-7.86 (m, 4 H), 7.72 (d, J = 8.1 Hz, 2 H), 7.47 (d, J = 8.1 Hz, 2 H), 5.08-5.00 (m, 1 H), 3.78 (dd, J = 3.7, 8.7 Hz, 2 H), 3.26-3.17 (m, 2 H), 1.80 (s, 3 H). 1 exchangeable proton not observed. LCMS (Method |
| | (S)-N-(1-cyano-2-(4′-cyano-[1,1′- | | BicarbBEHC18): [MH+] = 377 at |
| | biphenyl]-4-yl)ethyl)-3- | | 3.86 min. |
| | (methylthio)azetidine-3- | | |
| | carboxamide | | |
| 26 | 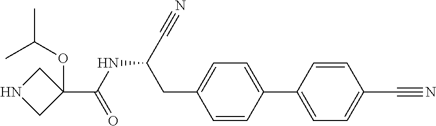 [SMILES: CC(C)OC1(C(=O)N[C@H](C#N)Cc2ccc(-c3ccc(C#N)cc3)cc2)CNC1] | F1 | ¹H NMR (400 MHz, DMSO): δ 8.67 (d, J = 8.1 Hz, 1 H), 7.99-7.95 (m, 4 H), 7.77 (d, J = 8.3 Hz, 2 H), 7.51 (d, J = 8.1 Hz, 2 H), 5.19-5.12 (m, 1 H), 3.68-3.55 (m, 2 H), 3.52- 3.44 (m, 3 H), 3.33-3.27 (m, 3 H), 1.02 (dd, J = 6.1, 14.4 Hz, 6 H). LCMS (method BicarbBEHC18): |
| | (S)-N-(1-cyano-2-(4′-cyano-[1,1′- | | [MH+] = 389 at 4.09 min. |
| | biphenyl]-4-yl)ethyl)-3- | | |
| | isopropoxyazetidine-3- | | |
| | carboxamide | | |

### Intermediate K1

### tert-Butyl (S)-3-((1-cyano-2-(4′-cyano-[1,1′-biphenyl]-4-yl)ethyl)carbamoyl)-3-(methylthio)azetidine-1-carboxylate

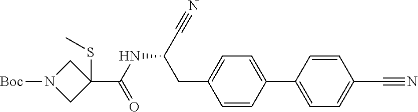 [SMILES: CSC1(C(=O)N[C@H](C#N)Cc2ccc(-c3ccc(C#N)cc3)cc2)CN(C(=O)OC(C)(C)C)C1]

To a solution of 3-(methylthio)azetidine-3-carboxylic acid hydrochloride (99 mg, 0.539 mmol) in methanol (2 mL) was added triethylamine (230 μL, 1.65 mmol) and di-tert-butyl dicarbonate (134 mg, 0.614 mmol). The resulting mixture was stirred at room temperature for 16 hours. The solvent was removed in vacuo. The residue was taken up with dichloromethane (6 mL) and N-(3-dimethylaminopropyl)-N′-ethylcarbodiimide hydrochloride (124 mg, 0.645 mmol) and 2-pyridinol 1-oxide (72 mg, 0.58 mmol) were added. The resulting mixture was stirred at room temperature for 30 min, then (S)-4′-(2-amino-2-cyanoethyl)-[1,1′-biphenyl]-4-carbonitrile (Intermediate F1, 133 mg, 0.54 mmol) was added followed by N,N-diisopropylethylamine (0.14 mL, 0.80 mmol). The mixture was stirred at room temperature for 16 hours. The mixture was diluted with dichloromethane (15 mL) and 2 N HCl (3 mL). The aqueous layer was extracted with dichloromethane (2×15 mL). The organic layers were combined, washed with saturated aqueous NaHCO₃, dried over MgSO₄, filtered, and concentrated in vacuo. The residue was purified by preparative HPLC to give the title compound (246 mg, 0.516 mmol, 95%).

¹H NMR (400 MHz, CDCl₃): δ 7.74 (d, J=8.8 Hz, 2H), 7.68 (d, J=8.5 Hz, 2H), 7.61 (d, J=8.2 Hz, 2H), 7.39 (d, J=8.2 Hz, 2H), 7.08 (d, J=8.5 Hz, 1H), 5.16 (q, J=7.4 Hz, 1H), 4.44 (br s, 1H), 4.26 (br s, 1H), 3.82 (dd, J=8.7, 17.1 Hz, 2H), 3.20 (d, J=7.0 Hz, 2H), 1.94 (s, 3H), 1.43 (s, 9H); LCMS (Method AcHSSC18): [MH+]=477 at 5.3 min.

### Example 19

### (S)—N-(1-cyano-2-(4′-cyano-[1,1′-biphenyl]-4-yl)ethyl)-3-(methylsulfonyl)azetidine-3-carboxamide

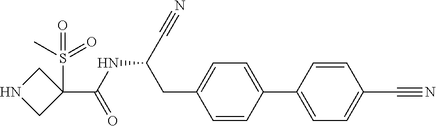 [SMILES: CS(=O)(=O)C1(C(=O)N[C@H](C#N)Cc2ccc(-c3ccc(C#N)cc3)cc2)CNC1]

To a solution of tert-butyl (S)-3-((1-cyano-2-(4′-cyano-[1,1′-biphenyl]-4-yl)ethyl)carbamoyl)-3-(methylthio)azetidine-1-carboxylate (Intermediate K1, 196 mg, 0.411 mmol, 1.00 eq) in dichloromethane (4 mL) was added 3-chloroperbenzoic acid (230 mg, 1.03 mmol, 2.50 eq, 77%) and the reaction mixture was stirred at room temperature for 16 hours. The reaction mixture was diluted with dichloromethane and washed with saturated sodium bicarbonate solution. The aqueous phase was extracted with dichloromethane. The combined organic phases were dried on MgSO₄, filtered and the solvent was concentrated in vacuo. The isolated material was stirred in formic acid (0.5 mL) at 50° C. for 10 min then allowed to cool to room temperature. The formic acid was removed in vacuo. The mixture was diluted with dichloromethane (15 mL) and shaken with saturated aqueous NaHCO₃ (5 mL). The organic layer was separated, and the aqueous layer was extracted with dichloromethane (2×15 mL). The combined organic phases were dried over MgSO₄, filtered, and concentrated in vacuo. The crude material was purified by reverse phase HPLC to give the title compound (24.0 mg, 60 μmol, 21%) as an off-white solid.

¹H NMR (400 MHz, DMSO): δ 9.26 (d, J=7.8 Hz, 1H), 7.92 (d, J=8.9 Hz, 4H), 7.89 (d, J=8.7 Hz, 4H), 7.73 (d, J=8.2 Hz, 2H), 7.49 (d, J=8.3 Hz, 2H), 5.12 (q, J=7.7 Hz, 1H), 3.94 (t, J=10.0 Hz, 2H), 3.79 (d, J=9.6 Hz, 1H), 3.70 (d, J=9.6 Hz, 1H), 3.28-3.16 (m, 2H), 2.86 (s, 3H), 2.78 (br s, 1H). LCMS (Method BicarbBEHC18): [MH+]=409.2 at 3.68 min.

### Intermediate L1

### 1-(tert-butyl) 3-methyl 3-bromoazetidine-1,3-dicarboxylate

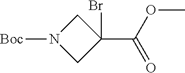 [SMILES: COC(=O)C1(Br)CN(C(=O)OC(C)(C)C)C1]

To a solution of 1 M lithium bis(trimethylsilyl)amide (5.6 mL, 5.57 mmol) in dry THE (12 mL) at −78° C. was added dropwise a solution of 1-(tert-butyl) 3-methylazetidine-1,3-dicarboxylate (0.9 mL, 4.65 mmol) in dry THE (12 mL) and the mixture was stirred at −78° C. for 15 min. A solution of tetrabromomethane (2311 mg, 6.97 mmol) in dry THE (12 mL) was added dropwise and the mixture was warmed to room temperature and stirred for 16 h. The reaction mixture was diluted with diethylether (100 mL), washed with saturated aqueous NaHCO₃ (2×80 mL) and brine (80 mL), filtered through a hydrophobic frit and the solvent was removed in vacuo. The residue was purified by column chromatography on silica gel, eluting with 10-30% diethyl ether in cyclohexane, to give the title compound (737 mg, 2.51 mmol, 54%) as a yellow oil.

¹H NMR (400 MHz, CDCl₃): δ 4.65 (dd, J=1.2, 10.4 Hz, 2H), 4.30 (dd, J=1.3, 10.4 Hz, 2H), 3.85 (s, 3H), 1.45 (s, 9H).

### Intermediate M1

### 1-(tert-butyl) 3-methyl 3-phenoxyazetidine-1,3-dicarboxylate

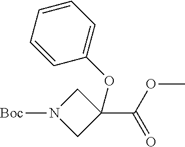 [SMILES: COC(=O)C1(Oc2ccccc2)CN(C(=O)OC(C)(C)C)C1]

To a mixture of 1-(tert-butyl) 3-methyl 3-bromoazetidine-1,3-dicarboxylate (Intermediate L1, 200 mg, 0.680 mmol) and potassium carbonate (517 mg, 3.74 mmol) in dry DMSO (5 mL) was added phenol (176 mg, 1.87 mmol) and the reaction mixture was stirred at 60° C. for 16 h. The reaction mixture was cooled to room temperature, diluted with water (50 mL) and extracted with EtOAc (3×75 mL). The combined organic phases were washed with brine (50 mL), filtered through a hydrophobic frit and concentrated in vacuo. The residue was purified by column chromatography on silica gel, eluting with 10-30% diethyl ether in cyclohexane to give the title compound (97 mg, 0.316 mmol, 46%) as an off white solid.

¹H NMR (400 MHz, CDCl₃): δ 7.30-7.25 (m, 2H), 7.00 (t, J=7.4 Hz, 1H), 6.61 (d, J=7.8 Hz, 2H), 4.47 (d, J=9.2 Hz, 2H), 4.16 (d, J=9.5 Hz, 2H), 3.79 (s, 3H), 1.45 (s, 9H); LCMS (Method AcHSSC18): [MH+]=308 at 5.35 min.

### Intermediate N1

### tert-butyl (S)-3-((1-cyano-2-(4′-cyano-[1,1′-biphenyl]-4-yl)ethyl)carbamoyl)-3-phenoxyazetidine-1-carboxylate

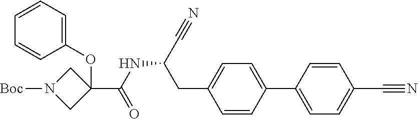 [SMILES: CC(C)(C)OC(=O)N1CC(Oc2ccccc2)(C(=O)N[C@H](C#N)Cc2ccc(-c3ccc(C#N)cc3)cc2)C1]

To 1-(tert-butyl) 3-methyl 3-phenoxyazetidine-1,3-dicarboxylate (Intermediate M1, 96 mg, 0.312 mmol) in 1:1 THE/water solution (3 mL) was added lithium hydroxide monohydrate (13 mg, 0.312 mmol, 1.00 eq) and the reaction mixture was stirred at room temperature for 16 h. The reaction mixture was diluted with water, acidified to pH 2 with 2 M HCl and extracted with DCM (2×10 mL). The combined organic phases were filtered through a hydrophobic frit and the solvent was removed in vacuo. The residue was dissolved in dichloromethane (2 mL), along with N-(3-dimethylaminopropyl)-N-ethylcarbodiimide hydrochloride (49 mg, 0.254 mmol) and 2-pyridinol 1-oxide (28 mg, 0.254 mmol) and stirred at room temperature for 30 min. 4-[4-[(2S)-2-Amino-2-cyano-ethyl]phenyl]benzonitrile (Intermediate F1, 52 mg, 0.211 mmol) was added followed by N,N-diisopropylethylamine (0.05 mL, 0.317 mmol) and the mixture was stirred for 16 h. The mixture was diluted with 1 M HCl and extracted with DCM (2×10 mL); the combined organic phases were washed with sat. aq. NaHCO₃, dried over MgSO₄, filtered and concentrated. The residue was purified by silica gel chromatography on silica gel eluting with 0-100% EtOAc in cyclohexane to give the tile compound (90 mg, 0.17 mmol, 55% over two steps) as a colourless oil.

¹H NMR (400 MHz, CDCl₃): δ 7.74 (d, J=8.4 Hz, 2H), 7.63 (d, J=8.4 Hz, 2H), 7.42 (d, J=8.2 Hz, 2H), 7.31 (dd, J=8.0, 8.0 Hz, 2H), 7.10-7.01 (m, 3H), 6.60 (d, J=8.0 Hz, 2H), 6.50 (d, J=8.9 Hz, 1H), 5.28-5.21 (m, 1H), 4.57-4.57 (m, 1H), 4.31-4.31 (m, 1H), 4.02-3.92 (m, 1H), 3.16-3.04 (m, 2H), 2.92 (dd, J=6.5, 13.9 Hz, 1H), 1.43 (s, 9H), 1.38 (d, J=4.9 Hz, 2H); LCMS (Method AcHSSC18): [MH-]=521 at 5.77 min.

### Example 21

### (S)—N-(1-cyano-2-(4′-cyano-[1,1′-biphenyl]-4-yl)ethyl)-3-phenoxyazetidine-3-carboxamide

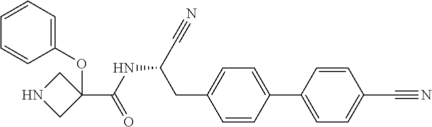 [SMILES: N#Cc1ccc(-c2ccc(C[C@@H](C#N)NC(=O)C3(Oc4ccccc4)CNC3)cc2)cc1]

A mixture of tert-butyl (S)-3-((1-cyano-2-(4′-cyano-[1,1′-biphenyl]-4-yl)ethyl)carbamoyl)-3-phenoxyazetidine-1-carboxylate (Intermediate N1, 90 mg, 0.172 mmol) in formic acid (0.3 mL) was stirred at 50° C. (pre-heated stirrer) for 10 min. The formic acid was removed in vacuo. The mixture was diluted with dichloromethane (15 mL) and shaken with saturated aqueous NaHCO₃ (5 mL). The organic layer was separated, and the aqueous layer was extracted with dichloromethane (2×15 mL). The combined organic phases were dried over MgSO₄, filtered, and concentrated in vacuo. The crude material was purified by reverse phase HPLC to give the title compound (5.3 mg, 12 μmol, 7%) as an off-white solid.

¹H NMR (400 MHz, DMSO): δ 8.88 (d, J=8.3 Hz, 1H), 7.92 (d, J=8.6 Hz, 2H), 7.84 (d, J=8.3 Hz, 2H), 7.58 (d, J=8.3 Hz, 2H), 7.28 (d, J=8.3 Hz, 2H), 7.23-7.18 (m, 2H), 6.90 (dd, J=7.5, 7.5 Hz, 1H), 6.52 (d, J=7.8 Hz, 2H), 5.08-5.01 (m, 1H), 3.86 (d, J=9.3 Hz, 1H), 3.68 (d, J=9.3 Hz, 1H), 3.50 (dd, J=9.1, 13.6 Hz, 2H), 3.17-3.11 (m, 2H); LCMS (Method BicarbBEHC18): [MH+]=423.6 at 4.38 min.

### Intermediate 01

### 3-Benzyl 1-(tert-butyl) azetidine-1,3-dicarboxylate

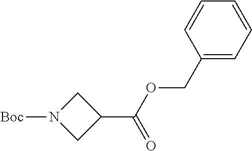 [SMILES: CC(C)(C)OC(=O)N1CC(C(=O)OCc2ccccc2)C1]

To a suspension of 1-(tert-butoxycarbonyl)azetidine-3-carboxylic acid (1.01 g, 5.02 mmol) in toluene (15 mL) at room temperature were successively added 1,8-diazabicyclo[5.4.0]undec-7-ene (1.1 mL, 7.50 mmol) and benzyl bromide (0.66 mL, 5.55 mmol). The solution was stirred at room temperature for 17 h. The reaction mixture was diluted with 20 mL of water and extracted with EtOAc (2×20 mL). The combined organics were washed with water (2×20 mL), dried on MgSO₄, filtered and concentrated. The residue was purified by column chromatography on silica gel, eluting with 0-100% EtOAc in cyclohexane to give the title compound (1.14 g, 3.91 mmol, 78%) as a colourless oil.

LCMS (method BicarbBEHC18): [MH+]=292 (ES+) at 5.04 min.

### Intermediate P1

### 3-Benzyl 1-(tert-butyl) 3-benzylazetidine-1,3-dicarboxylate

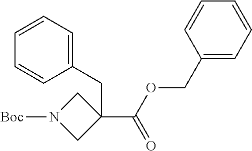 [SMILES: CC(C)(C)OC(=O)N1CC(Cc2ccccc2)(C(=O)OCc2ccccc2)C1]

A solution of 3-benzyl 1-(tert-butyl) azetidine-1,3-dicarboxylate (Intermediate 01, 273 mg, 0.937 mmol) was dissolved in tetrahydrofuran (3 mL) and cooled to −78° C. under nitrogen. A 1 M solution of lithium bis(trimethylsilyl)amide (1.4 mL, 1.40 mmol) was added dropwise over 60 s and stirring continued for 30 min. A solution of benzyl bromide (0.11 mL, 0.933 mmol) in tetrahydrofuran (1 mL) was added over 60 s and stirring was continued at −78° C. for a further 1 h. After this time the cooling bath was removed and the reaction was stirred at room temperature for 17 h. The reaction mixture was quenched by addition of saturated aqueous NH₄Cl (5 mL) and then extracted with EtOAc (50 mL). The aqueous layer was extracted with additional EtOAc (3×10 mL). The combined organic layers were washed with brine (2×5 mL), dried over MgSO₄, filtered and concentrated in vacuo. The residue was purified by silica gel chromatography eluting with 0-50% EtOAc in cyclohexane to give the title compound (107 mg, 0.28 mmol, 30%) as a colourless oil.

LCMS (method BicarbBEHC18): [MH+]=382 (ES+) at 5.86 min.

### Example 20

### (S)-3-Benzyl-N-(1-cyano-2-(4′-cyano-[1,1′-biphenyl]-4-yl)ethyl)azetidine-3-carboxamide

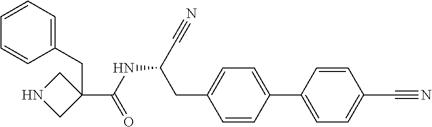 [SMILES: N#Cc1ccc(-c2ccc(C[C@@H](C#N)NC(=O)C3(Cc4ccccc4)CNC3)cc2)cc1]

A solution of 3-benzyl 1-(tert-butyl) 3-benzylazetidine-1,3-dicarboxylate (Intermediate P1, 107 mg, 0.281 mmol) in tetrahydrofuran (1 mL) and water (0.2 mL) was treated with lithium hydroxide monohydrate (31 mg, 0.748 mmol) and stirred for 17 h at room temperature. The mixture was concentrated to remove THF, then diluted in 5 mL water, acidified to pH 0 with 2 M HCl, and extracted with EtOAc (2×10 mL). The combined organics were passed through a hydrophobic frit and concentrated in vacuo. The residue was dissolved in DMF (1 mL) and HATU (118 mg, 0.310 mmol) was added, followed by 4-[4-[(2S)-2-amino-2-cyano-ethyl]phenyl]benzonitrile (Intermediate F1, 51 mg, 0.204 mmol) and N,N-diisopropylethylamine (0.11 mL, 0.614 mmol). The solution was stirred at room temperature for 6 days. The mixture was diluted with 10 mL EtOAc, washed with 10% citric acid (10 mL), saturated aqueous solution of NaHCO₃ (10 mL), water (10 mL) and brine (10 mL). The organic phase was dried on MgSO₄, filtered and the solvent was removed in vacuo. The isolated material was stirred in formic acid (1.5 mL) at 50° C. for 10 min then allowed to cool to room temperature. The formic acid was removed in vacuo. The mixture was diluted with dichloromethane (15 mL) and shaken with saturated aqueous NaHCO₃ (5 mL). The organic layer was separated, and the aqueous layer was extracted with dichloromethane (2×15 mL). The combined organic phases were dried over MgSO₄, filtered and concentrated in vacuo. The crude material was purified by reverse phase HPLC to give the title compound (10.8 mg, 25 μmol, 9% over three steps) as an off-white solid.

¹H NMR (400 MHz, DMSO): δ 8.55 (d, J=8.1 Hz, 1H), 7.91 (d, J=8.6 Hz, 2H), 7.86 (d, J=8.6 Hz, 2H), 7.73 (d, J=8.1 Hz, 2H), 7.47 (d, J=8.1 Hz, 2H), 7.12-7.09 (m, 3H), 6.91-6.87 (m, 2H), 5.04-4.97 (m, 1H), 3.51 (d, J=7.8 Hz, 2H), 3.28-3.04 (m, 6H). LCMS (Method AcHSSC18): [MH+]=421.7 at 3.61 min;

### Intermediate Q1

### 1′-(oxetan-3-yl)-6-(4,4,5,5-tetramethyl-1,3,2-dioxaborolan-2-yl)-3H-spiro[isobenzofuran-1,4′-piperidine]

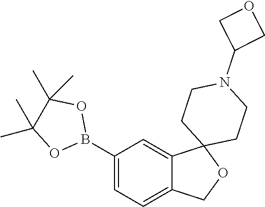 [SMILES: CC1(C)OB(c2ccc3c(c2)C2(CCN(C4COC4)CC2)OC3)OC1(C)C]

A reaction vessel was charged with 5-bromo-1′-(oxetan-3-yl)spiro[1H-isobenzofuran-3,4′-piperidine](prepared as described for in the patent WO2016038007A1, compound R₅, pag 63) (239 mg, 0.737 mmol), bis(pinacolato)diboron (225 mg, 0.885 mmol), potassium acetate (217 mg, 2.21 mmol) and [1,1′-bis(diphenylphosphino)ferrocene]dichloropalladium(II) complex with dichloromethane (55 mg, 0.0737 mmol) and the mixture was dissolved in cyclopentyl methyl ether (20 mL). The solution was degassed sparging with nitrogen for 20 minutes, then the vial was sealed and heated to 85° C. for 18 h. The solution was filtered through celite washing with abundant EtOAc, concentrated in vacuo and the crude was purified by chromatography on silica gel eluting with 0-100% EtOAc in cyclohexane to yield the title compound (185 mg, 0.498 mmol, 68%) as an off-white solid.

LCMS (Method AcHSSC18): [MH+]=372.3 at 3.39 min.

The following compound was prepared from commercially available 6-bromo-1-methyl-indolin-2-one following similar procedure as for Intermediate Q1 as reported in Table 7.

TABLE 7

| Intermediate | Structure & Name | Reagents | Analytical data |
|---|---|---|---|
| Q2 | 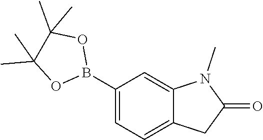 [SMILES: CN1C(=O)Cc2ccc(B3OC(C)(C)C(C)(C)O3)cc21] | 6-bromo-1- methylindolin- 2-one | ¹H NMR (400 MHz, CDCl₃): δ 7.53 (d, J = 7.05 Hz, 1 H), 7.28- 7.20 (m, 2 H), 3.53 (s, 2 H), 3.24 (s, 3 H), 1.36 (s, 12 H). |
| | 1-methyl-6-(4,4,5,5- | | |
| | tetramethyl-1,3,2- | | |
| | dioxaborolan-2-yl)indolin-2- | | |
| | one | | |
| Q3 | 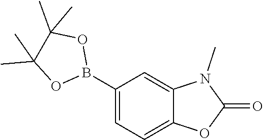 [SMILES: Cn1c(=O)oc2ccc(B3OC(C)(C)C(C)(C)O3)cc21] | 5-chloro-2- methylbenzo[d] oxazol-2(3H)- one | ¹H NMR (400 MHz, CDCl₃): δ 7.62 (d, J = 8.0 Hz, 1 H), 7.39 (s, 1 H), 7.20 (d, J = 7.9 Hz, 1 H), 3.42 (s, 3 H), 1.36 (s, 12 H). |
| | 3-methyl-5-(4,4,5,5- | | |
| | tetramethyl-1,3,2- | | |
| | dioxaborolan-2- | | |
| | yl)benzo[d]oxazol-2(3H)-one | | |

### Intermediate R1

### tert-butyl (S)-3-((2-(4-bromo-2-fluorophenyl)-1-cyanoethyl)carbamoyl)-3-methoxyazetidine-1-carboxylate

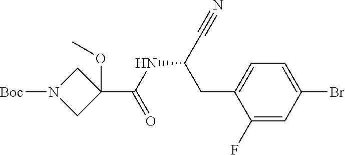 [SMILES: COC1(C(=O)N[C@H](C#N)Cc2ccc(Br)cc2F)CN(C(=O)OC(C)(C)C)C1]

A mixture of 1-tert-butoxycarbonyl-3-methoxy-azetidine-3-carboxylic acid (450 mg, 1.95 mmol), 2-pyridinol 1-oxide (540 mg, 4.86 mmol) and N-(3-dimethylaminopropyl)-N′-ethylcarbodiimide hydrochloride (933 mg, 4.86 mmol) in dichloromethane (3 mL) was stirred at room temperature for one hour, then (S)-2-amino-3-(4-bromo-2-fluorophenyl)propanenitrile (Intermediate F4, 473 mg, 1.95 mmol) and N,N-diisopropylethylamine (1017 μL, 5.84 mmol) were added. The resulting mixture was stirred at room temperature for 18 h. The reaction was diluted with DCM (50 mL) and washed with 1N HCl (50 mL), then saturated aqueous solution of NaHCO₃ (3×50 mL). The organic phase was dried over magnesium sulfate, filtered, and concentrated in vacuo to give a dense, brown oil. The crude was purified by chromatography on silica gel eluting with 0-30% EtOAc in cyclohexane to give the title compound (474 mg, 1.04 mmol, 53%) as an off-white foam.

¹H NMR (400 MHz, CDCl₃): δ 7.30 (dt, J=1.8, 7.8 Hz, 2H), 7.17 (t, J=8.0 Hz, 1H), 6.85 (d, J=8.5 Hz, 1H), 5.13 (dt, J=7.0, 8.8 Hz, 1H), 4.22 (d, J=9.5 Hz, 1H), 4.06 (d, J=10.0 Hz, 1H), 4.00 (d, J=10.0 Hz, 1H), 3.96 (d, J=9.5 Hz, 1H), 3.39 (s, 3H), 3.20 (dd, J=6.5, 14.0 Hz, 1H), 3.12 (dd, J=6.9, 14.0 Hz, 1H), 1.45 (s, 9H).

The following compound was prepared from commercially available Boc-azetidine-carboxylic acid and reagents reported below in Table 8 following similar procedures as for Intermediate R1.

TABLE 8

| Intermediate | Structure & Name | Reagents | Analytical data |
|---|---|---|---|
| R2 | 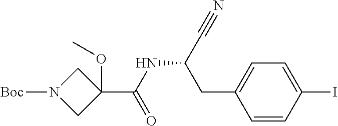 | F5 | LCMS (Method BicarbBEHC18): [MH−] = 484.2 at 5.2 min. |
| | tert-butyl (S)-3-((2-(4- | | |
| | iodophenyl)-1- | | |
| | cyanoethyl)carbamoyl)-3- | | |
| | methoxyazetidine-1-carboxylate | | |
| R3 | 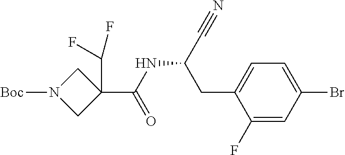 | F4 | LCMS (BicarbBEHC18): [MH−] = 474.1/476.1 at 5.18 min. |
| | tert-butyl (S)-3-((2-(4-bromo-2- | | |
| | fluorophenyl)-1- | | |
| | cyanoethyl)carbamoyl)-3- | | |
| | (difluoromethyl)azetidine-1- | | |
| | carboxylate | | |
| R4 | 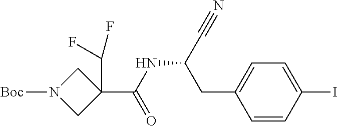 | F5 | LCMS (Method BicarbBEHC18): [MH−] = 504.2 at 5.37 min. |
| | tert-butyl (S)-3-((1-cyano-2-(4- | | |
| | iodophenyl)ethyl)carbamoyl)-3- | | |
| | (difluoromethyl)azetidine-1- | | |
| | carboxylate | | |

### Intermediate S1

### tert-butyl (S)-3-((1-cyano-2-(2-fluoro-4-(1′-(oxetan-3-yl)-311-spiro[isobenzofuran-1,4′-piperidin]-6-yl)phenyl)ethyl)carbamoyl)-3-(difluoromethyl)azetidine-1-carboxylate

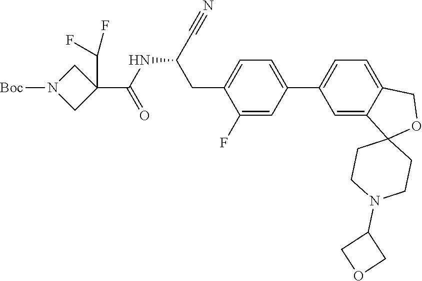 [SMILES: CC(C)(C)OC(=O)N1CC(C(=O)N[C@H](C#N)Cc2ccc(-c3ccc4c(c3)C3(CCN(C5COC5)CC3)OC4)cc2F)(C(F)F)C1]

A mixture of tert-butyl 3-[[(1S)-2-(4-bromo-2-fluoro-phenyl)-1-cyano-ethyl]carbamoyl]-3-(difluoromethyl)azetidine-1-carboxylate (Intermediate R₃, 70 mg, 0.147 mmol), 1′-(oxetan-3-yl)-6-(4,4,5,5-tetramethyl-1,3,2-dioxaborolan-2-yl)-3H-spiro[isobenzofuran-1,4′-piperidine](Intermediate Q1, 55 mg, 0.147 mmol), potassium carbonate (41 mg, 0.294 mmol) in 1,4-dioxane (2 mL)/water (0.1 mL) was degassed with N₂ then [1,1′-bis(diphenylphosphino)ferrocene]dichloropalladium(II) complex with dichloromethane (12 mg, 0.0147 mmol) was added and the mixture stirred at 80° C. for 2 hours. The mixture was cooled to room temperature, diluted with water, extracted with EtOAc (2×10 mL). The combined organic phases were dried with MgSO₄, filtered and concentrated in vacuo. The residue was purified by silica gel column chromatography, eluting with 0-100%˜EtOAc in cyclohexane, to afford the title compound (92 mg, 98% o) as an amber oil.

LCMS (Method BicarbBEHC18): [MH+]=641.6 at 5.11 min.

The following compound was prepared from reagents reported below in Table 9 following similar procedures as for Intermediate S1.

TABLE 9

| Intermediate | Structure & Name | Reagents | Analytical data |
|---|---|---|---|
| S2 | 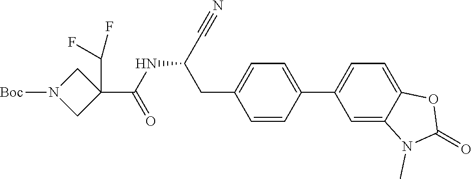 [SMILES: Cn1c(=O)oc2ccc(-c3ccc(C[C@@H](C#N)NC(=O)C4(C(F)F)CN(C(=O)OC(C)(C)C)C4)cc3)cc21] | R4 and. Q3 | LCMS (AcHSSC18): [MH−] = 525 at 5.01 min. |
| | tert-butyl (S)-3-((1-cyano-2-(4-(3- | | |
| | methyl-2-oxo-2,3- | | |
| | dihydrobenzo[d]oxazol-5- | | |
| | yl)phenyl)ethyl)carbamoyl)-3- | | |
| | (difluoromethyl)azetidine-1- | | |
| | carboxylate | | |
| S3 | 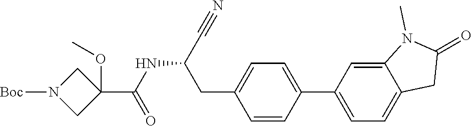 [SMILES: COC1(C(=O)N[C@H](C#N)Cc2ccc(-c3ccc4c(c3)N(C)C(=O)C4)cc2)CN(C(=O)OC(C)(C)C)C1] | R2 and Q2 | LCMS (Method BicarbBEHC18): [MH+] = 505.2 at 4.59 min. |
| | tert-butyl (S)-3-((1-cyano-2-(4-(1- | | |
| | methyl-2-oxoindolin-6- | | |
| | yl)phenyl)ethyl)carbamoyl)-3- | | |
| | methoxyazetidine-1-carboxylate | | |
| S4 | 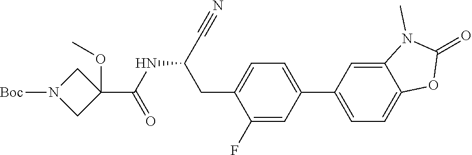 [SMILES: COC1(C(=O)N[C@H](C#N)Cc2ccc(-c3ccc4oc(=O)n(C)c4c3)cc2F)CN(C(=O)OC(C)(C)C)C1] | R1 and Q3 | LCMS (method BicarbBEHC18): [MH−] = 523.4 at 4.89 min. |
| | tert-butyl (S)-3-((1-cyano-2-(2- | | |
| | fluoro-4-(3-methyl-2-oxo-2,3- | | |
| | dihydrobenzo[d]oxazol-5- | | |
| | yl)phenyl)ethyl)carbamoyl)-3- | | |
| | methoxyazetidine-1-carboxylate | | |

### Example 14

### (S)—N-(1-cyano-2-(4-(3-methyl-2-oxo-2,3-dihydrobenzo[d]oxazol-5-yl)phenyl)ethyl)-3-(difluoromethyl)azetidine-3-carboxamide

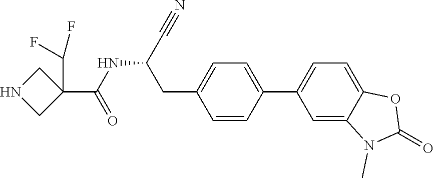 [SMILES: Cn1c(=O)oc2ccc(-c3ccc(C[C@@H](C#N)NC(=O)C4(C(F)F)CNC4)cc3)cc21]

A mixture of tert-butyl (S)-3-((1-cyano-2-(4-(3-methyl-2-oxo-2,3-dihydrobenzo[d]oxazol-5-yl)phenyl)ethyl)carbamoyl)-3-(difluoromethyl)azetidine-1-carboxylate (Intermediate S2, 30 mg, 0.057 mmol) in formic acid (0.5 mL) was stirred at 50° C. (pre-heated stirrer) for 10 min. The formic acid was removed in vacuo. The mixture was diluted with dichloromethane (15 mL) and shaken with saturated aqueous NaHCO₃ (5 mL). The organic layer was separated, and the aqueous layer was extracted with dichloromethane (2×15 mL). The combined organic phases were dried over MgSO₄, filtered, and concentrated in vacuo. The crude material was purified by reverse phase HPLC to give the title compound (10.0 mg, 23 μmol, 24%) as an off-white solid.

¹H NMR (400 MHz, DMSO): δ 8.95 (d, J=8.4 Hz, 1H), 7.68 (d, J=8.1 Hz, 2H), 7.58 (d, J=1.5 Hz, 1H), 7.44-7.37 (m, 4H), 6.28 (t, J=56.0 Hz, 1H), 5.06 (q, J=7.4 Hz, 1H), 3.69-3.60 (m, 4H), 3.40 (s, 3H), 3.18 (dd, J=3.4, 7.7 Hz, 2H). LCMS (Method AcHSSC18): [MH+]=427 at 3.14 min.

The following compounds were prepared from reagent reported below in Table 10 following similar procedures as for Example 14.

TABLE 10

| Example | Structure & Name | Reagents | Analytical data |
|---|---|---|---|
| 22 | 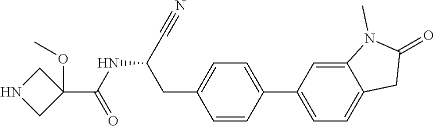 [SMILES: COC1(C(=O)N[C@H](C#N)Cc2ccc(-c3ccc4c(c3)N(C)C(=O)C4)cc2)CNC1] | S3 | ¹H NMR (400 MHz, DMSO): δ 8.76 (d, J = 8.4 Hz, 1 H), 7.65 (d, J = 8.2 Hz, 2 H), 7.39 (d, J = 8.2 Hz, 2 H), 7.33-7.27 (m, 2 H), 7.24 (s, 1 H), 5.06 (q, J = 8.2 Hz, 1 H), 3.62 (d, J = 8.8 Hz, 1 H), 3.57 (s, 2 H), 3.48- 3.34 (m, 3 H), 3.26-3.15 (m, 5 |
| | (S)-N-(1-cyano-2-(4-(1- | | H), 3.02 (s, 3 H). 1 |
| | methyl-2-oxoindolin-6- | | exchangeable proton not |
| | yl)phenyl)ethyl)-3- | | observed. LCMS (Method |
| | methoxyazetidine-3- | | BicarbBEHC18): [MH+] = |
| | carboxamide | | 405.2 at 3.31 min. |
| 36 | 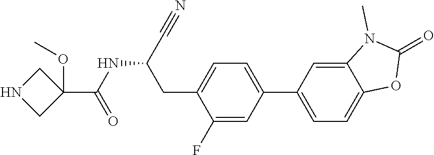 [SMILES: COC1(C(=O)N[C@H](C#N)Cc2ccc(-c3ccc4oc(=O)n(C)c4c3)cc2F)CNC1] | S4 | ¹H NMR (400 MHz, DMSO): δ 8.84 (d, J = 8.2 Hz, 1 H), 7.65 (d, J = 1.7 Hz, 1 H), 7.59 (dd, J = 1.8, 11.6 Hz, 1 H), 7.55 (dd, J = 1.8, 8.2 Hz, 1 H), 7.50-7.43 (m, 2 H), 7.40 (d, J = 8.4 Hz, 1 H), 5.09 (q, J = 7.8 Hz, 1 H), 3.63 (d, J = 8.8 Hz, 1 H), 3.49 (d, J = 8.8 Hz, 1 H), 3.45-3.17 (m, 4 H), 3.40 (s, |
| | (S)-N-(1-cyano-2-(2-fluoro-4- | | 3 H), 3.07 (s, 3 H). LCMS |
| | (3-methyl-2-oxo-2,3- | | (method AcHSSC18): [MH+] = |
| | dihydrobenzo[d]oxazol-5- | | 425.2 at 3.06 min. |
| | yl)phenyl)ethyl)-3- | | |
| | methoxyazetidine-3- | | |
| | carboxamide | | |

### Example 15

### (S)—N-(1-cyano-2-(2-fluoro-4-(1′-(oxetan-3-yl)-3H-spiro[isobenzofuran-1,4′-piperidin]-6-yl)phenyl)ethyl)-3-(difluoromethyl)azetidine-3-carboxamide

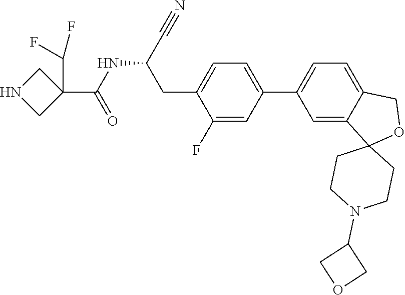 [SMILES: N#C[C@H](Cc1ccc(-c2ccc3c(c2)C2(CCN(C4COC4)CC2)OC3)cc1F)NC(=O)C1(C(F)F)CNC1]

A mixture of tert-butyl (S)-3-((1-cyano-2-(2-fluoro-4-(1′-(oxetan-3-yl)-3H-spiro[isobenzofuran-1,4′-piperidin]-6-yl)phenyl)ethyl)carbamoyl)-3-(difluoromethyl)azetidine-1-carboxylate (Intermediate S1, 69 mg, 0.108 mmol) and p-toluenesulfonic acid monohydrate (102 mg, 0.538 mmol) in acetonitrile (2 mL) was stirred at room temperature for 4 hours. The mixture was diluted with saturated aqueous solution of NaHCO₃, extracted with EtOAc (2×10 mL). Combined organic phases were dried on MgSO₄, filtered and concentrated in vacuo. The residue was purified by preparative HPLC to give the title compound (23 mg, 42 μmol, 39%) as an off-white solid.

¹H NMR (400 MHz, DMSO): δ 9.05 (d, J=8.1 Hz, 1H), 7.73 (s, 1H), 7.71-7.61 (m, 3H), 7.52 (dd, J=8.0, 8.0 Hz, 1H), 7.41 (d, J=7.8 Hz, 1H), 6.34 (t, J=56.5 Hz, 1H), 5.17 (q, J=7.5 Hz, 1H), 5.06 (s, 2H), 4.62 (dd, J=6.6, 6.6 Hz, 2H), 4.51 (dd, J=6.1, 6.1 Hz, 2H), 3.73-3.61 (m, 4H), 3.55-3.47 (m, 1H), 3.35-3.22 (m, 3H), 2.73-2.66 (m, 2H), 2.24-2.07 (m, 4H), 1.71 (d, J=11.9 Hz, 2H). LCMS (Method AcHSSC18): [MH+]=541.4 at 2.50 min.

### Intermediate T1

### tert-butyl (S)-3-((1-cyano-2-(2-fluoro-4-(1′-(oxetan-3-yl)-3H-spiro[isobenzofuran-1,4′-piperidin]-6-yl)phenyl)ethyl)carbamoyl)-3-methoxyazetidine-1-carboxylate

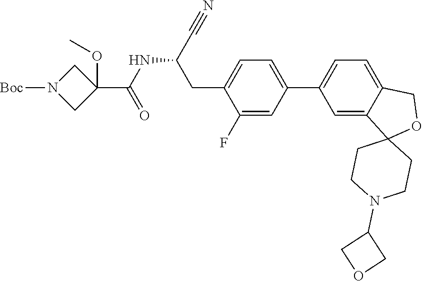 [SMILES: COC1(C(=O)N[C@H](C#N)Cc2ccc(-c3ccc4c(c3)C3(CCN(C5COC5)CC3)OC4)cc2F)CN(C(=O)OC(C)(C)C)C1]

A mixture of tert-butyl (S)-3-((2-(4-bromo-2-fluorophenyl)-1-cyanoethyl)carbamoyl)-3-methoxyazetidine-1-carboxylate (Intermediate R1, 0.130 g, 0.285 mmol), 1′-(oxetan-3-yl)-6-(4,4,5,5-tetramethyl-1,3,2-dioxaborolan-2-yl)-3H-spiro[isobenzofuran-1,4′-piperidine](Intermediate Q1, 0.120 g, 0.322 mmol) and aqueous 2M Na₂CO₃ (0.28 mL, 0.570 mmol) in acetonitrile (2 mL) was degassed under a nitrogen stream for 15 min. [1,1′-Bis(di-tert-butylphosphino)ferrocene]dichloropalladium(II) (0.020 g, 0.031 mmol) was added and the resulting mixture was degassed and heated at 85° C. for 4 h, then stirred at r.t. overnight. The mixture was partitioned between EtOAc and sat. NaHCO₃. The organic phase was dried over sodium sulfate, filtered and concentrated. The crude was purified by flash chromatography on silica gel cartridge (DCM to DCM:acetone=70:30) to obtain the title compound (0.115 g, 0.185 mmol, 65%) as a beige foam.

LCMS (method FormicNXC18): [MH+]=621.4 at 10.7 min.

### Example 53

### (S)—N-(1-cyano-2-(2-fluoro-4-(1′-(oxetan-3-yl)-3H-spiro[isobenzofuran-1,4′-piperidin]-6-yl)phenyl)ethyl)-3-methoxyazetidine-3-carboxamide

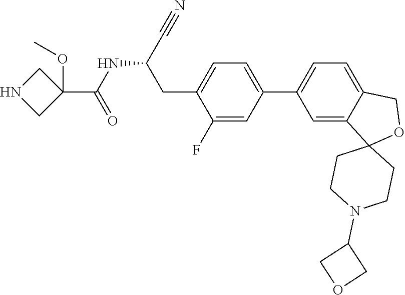 [SMILES: COC1(C(=O)N[C@H](C#N)Cc2ccc(-c3ccc4c(c3)C3(CCN(C5COC5)CC3)OC4)cc2F)CNC1]

To a solution of tert-butyl (S)-3-((1-cyano-2-(2-fluoro-4-(1′-(oxetan-3-yl)-3H-spiro[isobenzofuran-1,4′-piperidin]-6-yl)phenyl)ethyl)carbamoyl)-3-methoxyazetidine-1-carboxylate (Intermediate T1, 0.115 g, 0.185 mmol) in acetonitrile (3 mL), p-toluensulfonic acid monohydrate (0.176 g, 0.926 mmol) was added and the resulting mixture was stirred at r.t. overnight. The mixture was partitioned between EtOAc and sat. NaHCO₃. The aqueous phase was extracted with EtOAc and DCM and the combined organic layers were dried over sodium sulfate, filtered and concentrated. The crude was purified by flash chromatography on a 11 g Biotage NH cartridge (DCM to DCM:MeOH=97:3) to afford the title compound (0.050 g, 0.096 mmol, 52%) as a whitish foam.

¹H NMR (400 MHz, DMSO): δ 8.86 (d, J=8 Hz, 1H), 7.67 (s, 1H), 7.64-7.53 (m, 3H), 7.48-7.41 (m, 1H), 7.35 (d, J=8 Hz, 1H), 5.09 (q, J=7.5 Hz, 1H), 5.00 (s, 2H), 4.57 (t, J=6 Hz, 2H), 4.46 (t, J=6 Hz, 2H), 3.67 (d, J=7.5 Hz, 1H), 3.54-3.39 (m, 4H), 3.32-3.18 (m, 3H), 3.08 (s, 3H), 2.68-2.60 (m, 2H), 2.20-2.00 (m, 4H), 1.66 (d, J=12 Hz, 2H). LCMS (methodFormicNXC18): [MH+]=521.4 at 7.8 min.

### Example 37

### (S)—N-(1-cyano-2-(2-fluoro-4-(3-methyl-2-oxo-2,3-dihydrobenzo[d]oxazol-5-yl)phenyl)ethyl)-3-methoxy-1-methylazetidine-3-carboxamide 0

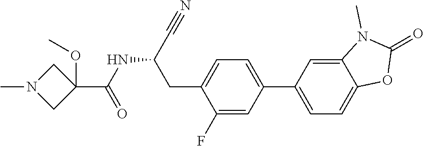 [SMILES: COC1(C(=O)N[C@H](C#N)Cc2ccc(-c3ccc4oc(=O)n(C)c4c3)cc2F)CN(C)C1]

A mixture of tert-butyl 3-[[(1S)-2-(4-bromo-2-fluoro-phenyl)-1-cyano-ethyl]carbamoyl]-3-methoxy-azetidine-1-carboxylate (Intermediate R₁, 200 mg, 0.438 mmol) and formic acid (1.7 mL, 43.8 mmol) was heated at 50° C. for 10 minutes. Formic acid was removed in vacuo and the residue was diluted in dichloromethane (15 mL), washed with saturated aqueous solution of NaHCO₃ (2×15 mL). The aqueous phase was extracted with DCM (2×10 mL). Combined organic phases were filtered through a phase separator frit and the solvent was removed in vacuo. The residue was dissolved in methanol (5 mL) and formaldehyde 37 wt % in H₂O (0.51 mL, 6.65 mmol) was added. The reaction mixture was stirred for 5 min. sodium acetate (116 mg, 1.42 mmol) and sodium cyanoborohydride (89 mg, 1.42 mmol) were added. The mixture was stirred for 72 hours at room temperature. The mixture was diluted with DCM (10 mL) and washed with saturated aqueous NaHCO₃ solution (3×10 mL) then brine. The organic phase was filtered through a phase separator frit. The solvent was removed in vacuo. The residue was dissolved in acetonitrile (2 mL) and water (0.5 mL). 3-methyl-5-(4,4,5,5-tetramethyl-1,3,2-dioxaborolan-2-yl)benzo[d]oxazol-2(3H)-one (Intermediate Q3, 137 mg, 0.499 mmol) and potassium phosphate tribasic monohydrate (204 mg, 0.886 mmol) were added and the mixture was degassed with nitrogen for 15 min. [1,1′-bis(diphenylphosphino)ferrocene]dichloropalladium(II) complex with dichloromethane (37 mg, 0.0443 mmol, 0.10 eq) was added and the reaction mixture was stirred at 85° C. for 18 h. The reaction was filtered through a pad of celite washing with abundant EtOAc and the solvents removed in vacuo. The residue was purified by preparative HPLC to give the title compound (42.4 mg, 96 μmol, 2200 over 3 steps).

¹H NMR (400 MHz, DMSO): δ 8.89 (d, J=8.4 Hz, 1H), 7.65 (d, J=1.7 Hz, 1H), 7.59 (dd, J=1.7, 11.7 Hz, 1H), 7.55 (dd, J=1.7, 8.0 Hz, 1H), 7.49-7.43 (m, 2H), 7.40 (d, J=8.4 Hz, 1H), 5.12-5.04 (N, 1H), 3.40 (s, 3H), 3.42-3.18 (m, 4H), 3.13 (d, J=8.1 Hz, 1H), 3.09 (d, J=8.0 Hz, 1H), 3.06 (s, 3H), 2.20 (s, 3H). LCMS (method AcHSSC18): [MH+]=439.2 at 3.10 m.

The following compounds were prepared from reagents reported below in Table 11 following similar procedures as for Example 37.

TABLE 11

| Ex. | Structure & Name | Reagent | Analytical data |
|---|---|---|---|
| 50 | 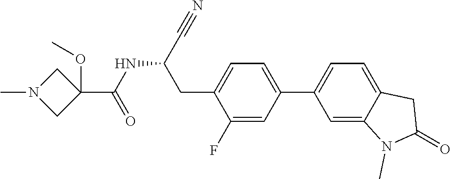 [SMILES: COC1(C(=O)N[C@H](C#N)Cc2ccc(-c3ccc4c(c3)N(C)C(=O)C4)cc2F)CN(C)C1] | R1 and Q2 | ¹H NMR (400 MHz, DMSO): δ 8.89 (d, J = 8.4 Hz, 1 H), 7.59 (dd, J = 1.7, 11.6 Hz, 1 H), 7.55 (dd, J = 1.7, 7.9 Hz, 1 H), 7.46 (t, J = 8.0 Hz, 1 H), 7.35-7.29 (m, 3 H), 5.12- 5.02 (m, 1 H), 3.58 (s, 2 H), 3.38 (d, J = 8.0 Hz, 1 H), 3.36-3.20 (m, 3 H), 3.19 (s, 3 H), 3.13 (d, J = 8.0 Hz, 1H ), 3.10 (d, J = 8.1 Hz, 1 H), 3.06 (s, 3 H), 2.20 (s, 3 H). LCMS (method AcHSSC18): [MH+] = |
| | (S)-N-(1-cyano-2-(2-fluoro-4- | | 437.2 at 2.92 min. |
| | (1-methyl-2-oxoindolin-6- | | |
| | yl)phenyl)ethyl)-3-methoxy-1- | | |
| | methylazetidine-3- | | |
| | carboxamide | | |
| 47 | 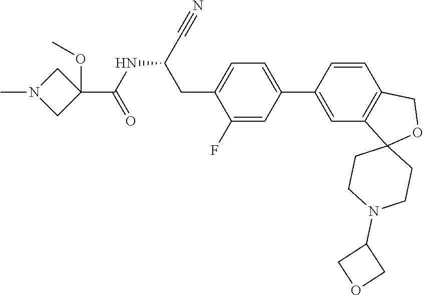 [SMILES: COC1(C(=O)N[C@H](C#N)Cc2ccc(-c3ccc4c(c3)C3(CCN(C5COC5)CC3)OC4)cc2F)CN(C)C1] | R1 and Q1 | ¹H NMR (400 MHz, CDCl₃): δ 7.47 (dd, J = 1.6, 7.8 Hz, 1 H), 7.41 (d, J = 8.7 Hz, 1 H), 7.36 (d, J = 3.1 Hz, 1 H), 7.35 (d, J = 0.9 Hz, 1 H), 7.31 (dd, J = 1.6, 6.1 Hz, 2 H), 7.28-7.23 (m, 1 H), 5.26-5.18 (m, 1 H), 5.11 (s, 2 H), 4.71 (t, J = 6.4 Hz, 2 H), 4.67 (t, J = 6.3 Hz, 2 H), 3.58 (p, J = 6.5 Hz, 1 H), 3.51-3.47 (m, 1 H), 3.46 (d, J = 8.1 Hz, 1 H), 3.37 (s, 3 H), 3.30 (dd, J = 3.1, 8.0 Hz, 2 H), 3.25 (dd, J = 6.8, 13.7 Hz, 2 H), 2.76-2.67 (m, 2 H), 2.38 (s, 3 H), 2.35-2.25 (m, 2 H), 2.06 (dt, J = 3.8, 13.5 Hz, 2 H), 1.90-1.80 (m, 2 H). LCMS (method AcHSSC18): [MH+] = 535.4 at 2.39 min. |
| | (S)-N-(1-cyano-2-(2-fluoro-4- | | |
| | (1′-(oxetan-3-yl)-3H- | | |
| | spiro[isobenzofuran-1,4′- | | |
| | piperidin]-6-yl)phenyl)ethyl)- | | |
| | 3-methoxy-1-methylazetidine- | | |
| | 3-carboxamide | | |

### Example 24

### (S)—N-(1-cyano-2-(4′-cyano-[1,1′-biphenyl]-4-yl)ethyl)-3-methoxy-1-methylazetidine-3-carboxamide

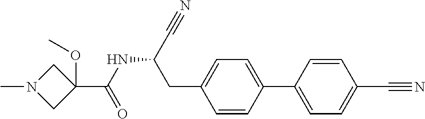 [SMILES: COC1(C(=O)N[C@H](C#N)Cc2ccc(-c3ccc(C#N)cc3)cc2)CN(C)C1]

To a solution of Example 6 (37 mg, 0.103 mmol) in methyl alcohol (2 mL) was added formaldehyde 37 wt % in H₂O (130 mg, 1.55 mmol) and the mixture was stirred for 5 minutes. Sodium acetate (27 mg, 0.329 mmol) and sodium cyanoborohydride 21 mg, 0.334 mmol) were added. The mixture was stirred for 16 hours at room temperature. The mixture was diluted with dichloromethane (10 mL) and washed with saturated aqueous solution of NaHCO₃ (3×10 mL) then brine. The organic phase was filtered through a phase separator frit. The solvent was removed in vacuo, the residue was taken up in DMSO (1.5 mL) and purified by preparative HPLC to give the title compound as an off-white solid (15 mg, 0.039 mmol, 38% yield).

¹H NMR (400 MHz, DMSO): δ 8.80 (d, J=8.6 Hz, 1H), 7.91 (d, J=8.3 Hz, 2H), 7.86 (d, J=8.6 Hz, 2H), 7.71 (d, J=8.1 Hz, 2H), 7.43 (d, J=8.3 Hz, 2H), 5.10-5.03 (m, 1H), 3.36 (d, J=7.8 Hz, 1H), 3.22 (dd, J=7.7, 7.7 Hz, 3H), 3.07 (dd, J=8.0, 20.8 Hz, 2H), 2.99 (s, 3H), 2.18 (s, 3H). LCMS (method AcHSSC18): [MH+]=375 at 3.26 min.

The following compounds were prepared from reagents reported below in Table 12 following similar procedures as for Example 24.

TABLE 12

| Ex. | Structure & Name | Reagent | Analytical data |
|---|---|---|---|
| 41 | 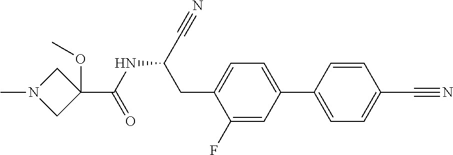 [SMILES: COC1(C(=O)N[C@H](C#N)Cc2ccc(-c3ccc(C#N)cc3)cc2F)CN(C)C1] | Example 12 | ¹H NMR (400 MHz, DMSO): δ 8.89 (d, J = 8.3 Hz, 1 H), 7.93 (s, 4 H), 7.66 (dd, J = 1.8, 11.4 Hz, 1 H), 7.61 (dd, J = 1.9, 8.0 Hz, 1 H), 7.51 (dd, J = 8.0, 8.0 Hz, 1 H), 5.12-5.05 (m, 1 H), 3.42- 3.35 (m, 1 H), 3.29-3.19 (m, 3 H), 3.10 (dd, J = 8.1, 14.7 Hz, 2 H), 3.05 (s, 3 H), 2.20 (s, 3 H). LCMS (Method |
| | (S)-N-(1-cyano-2-(4′-cyano-3- | | BicarbBEHC18): [MH+] = |
| | fluoro-[1,1′-biphenyl]-4- | | 393.2 at 4.18 min. |
| | yl)ethyl)-3-methoxy-1- | | |
| | methylazetidine-3-carboxamide | | |
| 35 | 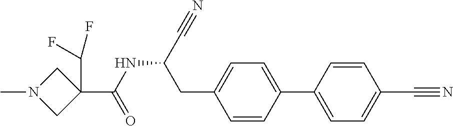 [SMILES: CN1CC(C(=O)N[C@H](C#N)Cc2ccc(-c3ccc(C#N)cc3)cc2)(C(F)F)C1] | Example 7 | ¹H NMR (400 MHz, DMSO): δ 8.90 (d, J = 7.8 Hz, 1 H), 7.94- 7.86 (m, 4 H), 7.73 (d, J = 8.3 Hz, 2 H), 7.45 (d, J = 8.3 Hz, 2 H), 6.23 (t, J = 55.4 Hz, 1 H), 5.12-5.06 (m, 1 H), 3.27-3.20 (m, 6 H), 2.14 (s, 3 H). LCMS (method AcHSSC18): [MH+] = 395.2 at 3.42 min. |
| | (S)-N-(1-cyano-2-(4′-cyano- | | |
| | [1,1′-biphenyl]-4-yl)ethyl)-3- | | |
| | (difluoromethyl)-1- | | |
| | methylazetidine-3-carboxamide | | |
| 40 | 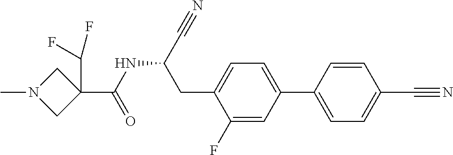 [SMILES: CN1CC(C(=O)N[C@H](C#N)Cc2ccc(-c3ccc(C#N)cc3)cc2F)(C(F)F)C1] | Example 13 | ¹H NMR (400 MHz, DMSO): δ 8.99 (d, J = 7.8 Hz, 1 H), 7.94 (s, 4 H), 7.68 (dd, J = 1.8, 11.4 Hz, 1 H), 7.62 (dd, J = 1.9, 8.0 Hz, 1 H), 7.53 (dd, J = 8.0, 8.0 Hz, 1 H), 6.24 (t, J = 56.0 Hz, 1 H), 5.17-5.10 (m, 1 H), 3.28-3.22 (m, 6 H), 2.16 (s, 3 H). LCMS (Method BicarbBEHC18): [MH+] = 413.2 at 4.42 min. |
| | (S)-N-(1-cyano-2-(4′-cyano-3- | | |
| | fluoro-[1,1′-biphenyl]-4- | | |
| | yl)ethyl)-3-(difluoromethyl)-1- | | |
| | methylazetidine-3-carboxamide | | |
| 38 | 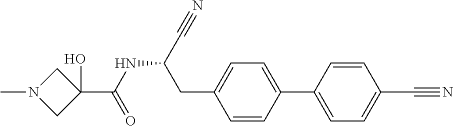 [SMILES: CN1CC(O)(C(=O)N[C@H](C#N)Cc2ccc(-c3ccc(C#N)cc3)cc2)C1] | Example 1 | 1H NMR (400 MHz, DMSO): δ 8.67 (d, J = 8.4 Hz, 1 H), 7.97 (d, J = 8.6 Hz, 2 H), 7.93 (d, J = 8.6 Hz, 2 H), 7.68 (d, J = 8.3 Hz, 2 H), 7.47 (d, J = 8.3 Hz, 2 H), 6.44 (s, 1 H), 5.08 (dd, J = 8.1, 16.0 Hz, 1 H), 3.47 (d, J = 7.6 Hz, 1 H), 3.27 (dd, J = 6.2, |
| | (S)-N-(1-cyano-2-(4′-cyano- | | 6.7 Hz, 2 H), 3.12 (dd, J = 7.6, |
| | [1,1′-biphenyl]-4-yl)ethyl)-3- | | 13.2 Hz, 2 H), 2.25 (s, 3H). |
| | hydroxy-1-methylazetidine-3- | | LCMS (method AcHSSC18): |
| | carboxamide | | [MH+] = 361.1 at 3.08 min |
| 43 | 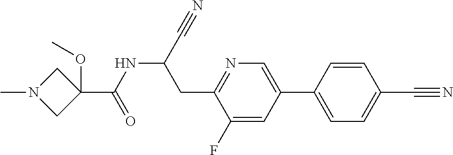 [SMILES: COC1(C(=O)NC(C#N)Cc2ncc(-c3ccc(C#N)cc3)cc2F)CN(C)C1] | Example 42 | ¹H NMR (400 MHz, DMSO): δ 8.94 (d, J = 8.3 Hz, 1 H), 8.87- 8.86 (m, 1 H), 8.22 (dd, J = 1.9, 11.0 Hz, 1 H), 8.05-7.97 (m, 4 H), 5.40-5.36 (m, 1 H), 3.59- 3.51 (m, 1 H), 3.44-3.33 (m, 3 H), 3.17-3.12 (m, 2 H), 3.10 (s, 3 H), 2.22 (s, 3 H). LCMS (method AcHSSC18): [MH+] = 394.2 at 2.94 min. |
| | N-(1-cyano-2-(5-(4- | | |
| | cyanophenyl)-3-fluoropyridin-2- | | |
| | yl)ethyl)-3-methoxy-1- | | |
| | methylazetidine-3-carboxamide | | |
| 39 | 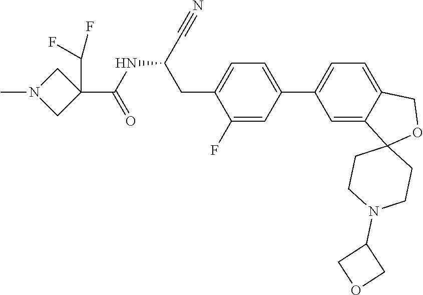 [SMILES: CN1CC(C(=O)N[C@H](C#N)Cc2ccc(-c3ccc4c(c3)C3(CCN(C5COC5)CC3)OC4)cc2F)(C(F)F)C1] | Example 15 | ¹H NMR (400 MHz, DMSO): δ 9.05 (d, J = 7.8 Hz, 1H), 7.72 (s, 1H), 7.70-7.61 (m, 3H), 7.52 (dd, J = 8.1, 8.1 Hz, 1H), 7.41 (d, J = 7.8 Hz, 1H), 6.31 (t, J = 56.1 Hz, 1H), 5.17 (q, J = 7.8 Hz, 1H), 5.06 (s, 2H), 4.62 (dd, J = 6.4, 6.4 Hz, 2H), 4.51 (dd, J = 6.1, 6.1 Hz, 2H), 3.55-3.45 (m, 1H), 3.34-3.25 (m, 6H), 2.73-2.67 (m, 2H), 2.22-2.21 (m, 7H), 1.71 (d, J = 12.1 Hz, 2H). LCMS (Method AcHSSC18): [MH+] = 555.2 at 2.48 min. |
| | (S)-N-(1-cyano-2-(2-fluoro-4- | | |
| | (1′-(oxetan-3-yl)-3H- | | |
| | spiro[isobenzofuran-1,4′- | | |
| | piperidin]-6-yl)phenyl)ethyl)-3- | | |
| | (difluoromethyl)-1- | | |
| | methylazetidine-3-carboxamide | | |

### Example 46

### (S)—N-(1-cyano-2-(4′-cyano-[1,1′-biphenyl]-4-yl)ethyl)-3-methoxy-1-propylazetidine-3-carboxamide

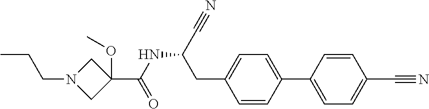 [SMILES: CCCN1CC(OC)(C(=O)N[C@H](C#N)Cc2ccc(-c3ccc(C#N)cc3)cc2)C1]

To a solution of Example 6 (50 mg, 0.139 mmol) and propionaldehyde (150 μL, 2.08 mmol) were dissolved in methyl alcohol (10 mL) and stirred for 5 minutes. Sodium acetate (36 mg, 0.444 mmol) and sodium cyanoborohydride (28 mg, 0.444 mmol) were added and the reaction mixture stirred at room temperature for 72 h. The reaction was diluted with DCM (20 mL) and sat NaHCO₃ (15 mL) and phases separated. The organic layer was further washed with NaHCO₃ (2×10 mL) and the combined aqueous backwashed with DCM (10 mL). Combined organics were dried through a phase separator and solvents removed in vacuo. The crude was dissolved in DMSO and purified by preparative HPLC to give the title compound (16 mg, 0.0392 mmol, 28%) as an off-white solid.

¹H NMR (400 MHz, DMSO): δ 8.80 (d, J=8.4 Hz, 1H), 7.91 (d, J=8.6 Hz, 2H), 7.86 (d, J=8.6 Hz, 2H), 7.71 (d, J=8.3 Hz, 2H), 7.44 (d, J=8.3 Hz, 2H), 5.12-5.04 (m, 1H), 3.35-3.13 (m, 4H, partly covered by water residual peak), 3.07 (d, J=8.2 Hz, 1H), 3.03-2.98 (m, 1H), 3.00 (s, 3H), 2.27 (t, J=7.2 Hz, 2H), 1.20 (hex, J=7.3 Hz, 2H), 0.77 (t, J=7.5 Hz, 3H). LCMS (method BicardBEHC18): [MH+]=403.2 at 4.63 min.

The following compounds were prepared from appropriate commercially available aldehydes or ketones and reagents reported below in Table 13 following similar procedures, as for Example 46.

TABLE 13

| Ex. | Structure & Name | Reagent | Analytical data |
|---|---|---|---|
| 30 | 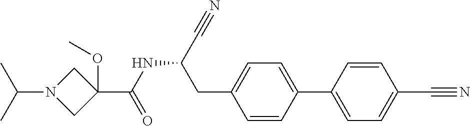 [SMILES: COC1(C(=O)N[C@H](C#N)Cc2ccc(-c3ccc(C#N)cc3)cc2)CN(C(C)C)C1] | Example 6 | ¹H NMR (400 MHz, DMSO): δ 8.82 (d, J = 8.4 Hz, 1 H), 7.91 (d, J = 8.7 Hz, 2 H), 7.86 (d, J = 8.7 Hz, 2 H), 7.71 (d, J = 8.3 Hz, 2 H), 7.44 (d, J = 8.3 Hz, 2 H), 5.13-5.04 (m, 1 H), 3.36-3.10 (m, 4 H), 3.05-2.93 (m, 2 H), 2.99 (s, 3 H), 2.20 (hept, J = 6.2 Hz, 1 H), 0.78 (d, J = 6.2 Hz, 6 |
| | (S)-N-(1-cyano-2-(4′-cyano- | | H). LCMS (method AcHSSC18): |
| | [1,1′-biphenyl]-4-yl)ethyl)-1- | | [MH+] = 403.2 at 3.33 min. |
| | isopropyl-3-methoxyazetidine- | | |
| | 3-carboxamide | | |
| 51 | 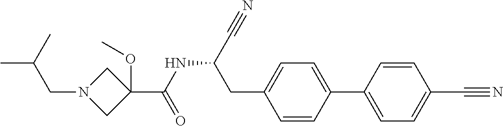 [SMILES: COC1(C(=O)N[C@H](C#N)Cc2ccc(-c3ccc(C#N)cc3)cc2)CN(CC(C)C)C1] | Example 6 | ¹H NMR (400 MHz, DMSO): δ 8.79 (d, J = 8.4 Hz, 1 H), 7.91 (d, J = 8.6 Hz, 2 H), 7.86 (d, J = 8.6 Hz, 2 H), 7.71 (d, J = 8.3 Hz, 2 H), 7.43 (d, J = 8.3 Hz, 2 H), 5.13-5.04 (m, 1 H), 3.53-2.99 (m, 6 H), 3.01 (s, 3 H), 2.13 (d, J = 6.9 Hz, 2 H), 1.42 (p, J = 6.9 Hz, 1 H), 0.77 (d, J = 6.9 Hz, 3 |
| | (S)-N-(1-cyano-2-(4′-cyano- | | H), 0.74 (d, J = 6.9 Hz, 3 H). LCMS |
| | [1,1′-biphenyl]-4-yl)ethyl)-1- | | (method BicarbBEHC18): [MH+] = |
| | isobutyl-3-methoxyazetidine-3- | | 417.2 at 4.99 min. |
| | carboxamide | | |
| 52 | 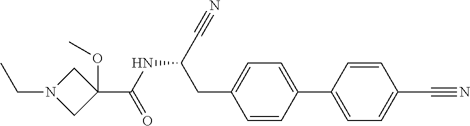 [SMILES: CCN1CC(OC)(C(=O)N[C@H](C#N)Cc2ccc(-c3ccc(C#N)cc3)cc2)C1] | Example 6 | ¹H NMR (400 MHz, DMSO): δ 8.81 (d, J = 8.3 Hz, 1 H), 7.91 (d, J = 8.7 Hz, 2 H), 7.86 (d, J = 8.7 Hz, 2 H), 7.71 (d, J = 8.3 Hz, 2 H), 7.44 (d, J = 8.3 Hz, 2 H), 5.12-5.03 (m, 1 H), 3.35-3.15 (m, 4 H), 3.04 (d, J = 8.0 Hz, 1 H), 3.01-2.96 (m, 1 H), 2.99 (s, 3 H), 2.33 (q, J = 7.2 Hz, 2 H), 0.80 |
| | (S)-N-(1-cyano-2-(4′-cyano- | | (t, J = 7.2 Hz, 3 H). LCMS (method |
| | [1,1′-biphenyl]-4-yl)ethyl)-1- | | AcHSSC18): [MH+] = 389.2 at 3.25 |
| | ethyl-3-methoxyazetidine-3- | | min. |
| | carboxamide | | |
| 29 | 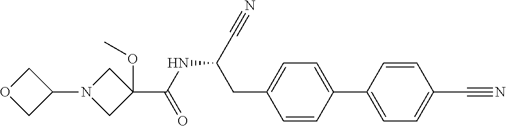 [SMILES: COC1(C(=O)N[C@H](C#N)Cc2ccc(-c3ccc(C#N)cc3)cc2)CN(C2COC2)C1] | Example 6 | ¹H NMR (400 MHz, DMSO): δ 8.87 (d, J = 8.4 Hz, 1 H), 7.91 (d, J = 8.6 Hz, 2 H), 7.86 (d, J = 8.6 Hz, 2 H), 7.71 (d, J = 8.3 Hz, 2 H), 7.44 (d, J = 8.3 Hz, 2 H), 5.14-5.05 (m, 1 H), 4.50 (td, J = 2.4, 6.6 Hz, 2 H), 4.30- 4.24 (m, 2 H), 3.72-3.65 (m, 1 H), 3.49-3.15 (m, 6 H), 3.02 (s, 3 H). |
| | (S)-N-(1-cyano-2-(4′-cyano- | | LCMS (method BicarbBEHC): |
| | [1,1′-biphenyl]-4-yl)ethyl)-3- | | [MH+] = 417.2 at 4.03 min. |
| | methoxy-1-(oxetan-3- | | |
| | yl)azetidine-3-carboxamide | | |
| 27 | 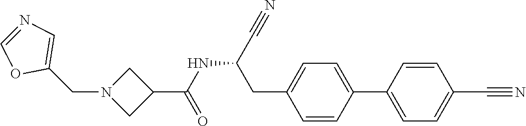 [SMILES: N#Cc1ccc(-c2ccc(C[C@@H](C#N)NC(=O)C3CN(Cc4cnco4)C3)cc2)cc1] | Example 9 | ¹H NMR (400 MHz, DMSO): δ 8.78 (d, J = 7.8 Hz, 1 H), 8.30 (s, 1 H), 7.95 (q, J = 8.8 Hz, 4 H), 7.78 (d, J = 8.3 Hz, 2 H), 7.48 (d, J = 8.3 Hz, 2 H), 7.03 (s, 1 H), 5.08-5.01 (m, 1 H), 3.59 (s, 2 H), 3.44-3.40 (m, 1 H), 3.24-3.08 (m, 6 H). LCMS (method BicarbBEHC18): [MH+] = 412 at |
| | (S)-N-(1-cyano-2-(4′-cyano- | | 3.89 min. |
| | [1,1′-biphenyl]-4-yl)ethyl)-1- | | |
| | (oxazol-5-ylmethyl)azetidine- | | |
| | 3-carboxamide | | |
| 25 | 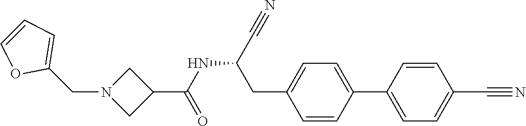 [SMILES: N#Cc1ccc(-c2ccc(C[C@@H](C#N)NC(=O)C3CN(Cc4ccco4)C3)cc2)cc1] | Example 9 | ¹H NMR (400 MHz, DMSO): δ 8.73 (d, J = 7.8 Hz, 1 H), 7.97-7.89 (m, 4 H), 7.75 (d, J = 8.3 Hz, 2 H), 7.56 (d, J = 1.0 Hz, 1 H), 7.46 (d, J = 8.3 Hz, 2 H), 6.37 (dd, J = 1.9, 3.2 Hz, 1 H), 6.21 (d, J = 2.5 Hz, 1 H), 5.06-4.99 (m, 1 H), 3.47 (s, 2 H), 3.23-3.03 (m, 7 H). LCMS (method AcHSSC18): |
| | (S)-N-(1-cyano-2-(4′-cyano- | | [MH+] = 411 at 3.49 min. |
| | [1,1′-biphenyl]-4-yl)ethyl)-1- | | |
| | (furan-2-ylmethyl)azetidine-3- | | |
| | carboxamide | | |

### Example 23

### (S)—N-(1-cyano-2-(4′-cyano-[1,1′-biphenyl]-4-yl)ethyl)-1-(oxetan-3-yl)azetidine-3-carboxamide

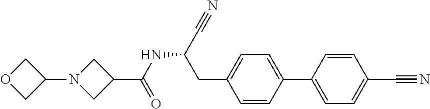 [SMILES: N#Cc1ccc(-c2ccc(C[C@@H](C#N)NC(=O)C3CN(C4COC4)C3)cc2)cc1]

A mixture of 3-azetidinecarboxylic acid (300 mg, 2.97 mmol), 3-oxetanone (209 μL, 3.26 mmol) and acetic acid (170 μL, 2.97 mmol) in dichloromethane (4 mL) was stirred at room temperature for 2 hours then sodium triacetoxyborohydride (1006 mg, 4.75 mmol) was added. The resulting mixture was stirred at room temperature for 16 hours then the mixture was diluted with dichloromethane (10 mL) and washed with saturated aqueous solution of NaHCO₃. Organic phase was collected, filtered through a phase separator frit. Solvent was removed in vacuo. The residue was dissolved in dichloromethane (5 mL) then 2-pyridinol 1-oxide (42 mg, 0.379 mmol) and N-(3-dimethylaminopropyl)-N′-ethylcarbodiimide hydrochloride (75 mg, 0.392 mmol) were added. The resulting mixture was stirred at room temperature for one hour, then (S)-4′-(2-amino-2-cyanoethyl)-[1,1′-biphenyl]-4-carbonitrile (Intermediate F1, 75 mg, 0.303 mmol) and N,N-Diisopropylethylamine (90 μL, 0.518 mmol) were added. The resulting mixture was stirred at room temperature overnight. The reaction was diluted with dichloromethane (20 mL) and washed with 1N HCl (10 mL) then saturated aqueous solution of NaHCO₃ (2×10 mL). The organic phase was dried over magnesium sulfate, filtered and concentrated in vacuo. Residue was purified by preparative HPLC to give the title compound (2.1 mg, 5.40 μmol, 2%) as an off-white solid.

¹H NMR (400 MHz, DMSO): δ 8.74 (d, J=7.6 Hz, 1H), 7.94-7.86 (m, 4H), 7.73 (d, J=8.1 Hz, 2H), 7.43 (d, J=8.1 Hz, 2H), 5.04-4.98 (m, 1H), 4.49 (dd, J=6.2, 6.2 Hz, 2H), 4.29 (dd, J=5.8, 5.8 Hz, 2H), 3.64-3.57 (m, 1H), 3.36-3.35 (m, 1H), 3.21-3.04 (m, 6H). LCMS (method BicarbBEHC18): [MH+]=387 at 3.84 min.

### Example 28

### (S)—N-(1-cyano-2-(4′-cyano-[1,1′-biphenyl]-4-yl)ethyl)-1-(2-(methylamino)-2-oxoethyl)azetidine-3-carboxamide

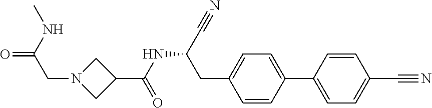 [SMILES: CNC(=O)CN1CC(C(=O)N[C@H](C#N)Cc2ccc(-c3ccc(C#N)cc3)cc2)C1]

To a solution of Example 9 (55 mg, 0.166 mmol) in N,N-dimethylformamide (1 mL) was successively added triethylamine (70 μL, 0.502 mmol) and 2-bromo-N-methyl-acetamide (25 mg, 0.166 mmol). The resulting mixture was heated at 60° C. for three hours, then the solvent was removed in vacuo. The residue was partitioned between dichloromethane (10 mL) and saturated aqueous solution of NaHCO₃ (10 mL). The organic phase was collected, washed with water (2×10 mL), brine (10 mL) and filtered through a phase separator frit. The solvent was removed in vacuo. The residue was purified by preparative HPLC to give the title compound (4.9 mg, 0.0111 mmol, 7%).

¹H NMR (400 MHz, DMSO): δ 8.72 (d, J=7.8 Hz, 1H), 7.94-7.86 (m, 4H), 7.73 (d, J=8.3 Hz, 2H), 7.56 (d, J=3.8 Hz, 1H), 7.43 (d, J=8.1 Hz, 2H), 5.00 (q, J=7.7 Hz, 1H), 3.45-3.39 (m, 2H), 3.19-3.03 (m, 5H), 2.93 (s, 2H), 2.55 (d, J=4.7 Hz, 3H). LCMS (method BicarbBEHC18): [MH+]=402 at 3.78 min.

### Example 44 and Example 45

### Single enantiomers of N-(1-cyano-2-(5-(4-cyanophenyl)-3-fluoropyridin-2-yl)ethyl)-3-methoxy-1-methylazetidine-3-carboxamide (enantiomers 1 and 2)

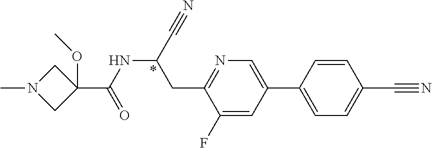 [SMILES: COC1(C(=O)NC(C#N)Cc2ncc(-c3ccc(C#N)cc3)cc2F)CN(C)C1]

Purification of the 1:1 mixture of enantiomers of Example 43 (26 mg) by chiral preparative SFC afforded the single enantiomers.

Title compound (Example 44, single enantiomer 1) was obtained as an off-white solid (5.6 mg, 43%).

¹H NMR (400 MHz, DMSO): δ 8.95 (d, J=8.0 Hz, 1H), 8.88 (s, 1H), 8.23 (dd, J=1.9, 10.9 Hz, 1H), 8.02 (q, J=8.7 Hz, 4H), 5.40-5.33 (m, 1H), 3.56 (ddd, J=1.7, 7.8, 15.2 Hz, 1H), 3.45-3.34 (i, 3H), 3.15 (dd, J=8.1, 11.5 Hz, 2H), 3.11 (s, 3H), 2.24 (s, 3H). LCMS (method AcHSSC18): [MH+]=394.2 at 2.89 min. Chiral analysis (method 1): [MH+]=394.2 at 3.00 min

Title compound (Example 45, single enantiomer 2) was obtained as an off-white solid (6.1 mg, 470%).

¹H NMR (400 MHz, DMSO): δ 8.95 (d, J=8.3 Hz, 1H), 8.88 (s, 1H), 8.23 (dd, J=1.9, 10.9 Hz, 1H), 8.02 (q, J=8.7 Hz, 4H), 5.40-5.34 (m, 1H), 3.56 (ddd, J=1.7, 7.8, 15.2 Hz, 1H), 3.44-3.30 (m, 3H), 3.15 (dd, J=8.2, 11.5 Hz, 2H), 3.10 (s, 3H), 2.23 (s, 3H). LCMS (method AcHSSC18): [MH+]=394.2 at 2.92 m-no Chiral analysis (method 1): [MH+]=394.2 at 4.70 min

Similarly, the following compounds were prepared from Example 31 as reported below in Table 14 by chiral preparative SFC.

TABLE 14

| Ex. | Structure & Name | Reagent | Analytical data |
|---|---|---|---|
| 32 | 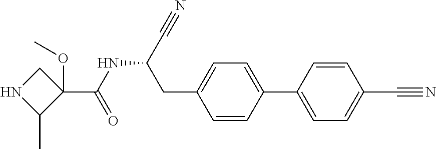 [SMILES: COC1(C(=O)N[C@H](C#N)Cc2ccc(-c3ccc(C#N)cc3)cc2)CNC1C] | Example 31 | LCMS (method AcHSSC18): [MH+] = 375.4 at 3.23 min. Chiral analysis (method 2): [MH+] = 375.4 at 2.00 min. |
| | diastereoisomer 1 of N-((S)-1-cyano-2- | | |
| | (4′-cyano-[1,1′-biphenyl]-4-yl)ethyl)-3- | | |
| | methoxy-2-methylazetidine-3- | | |
| | carboxamide | | |
| 33 | 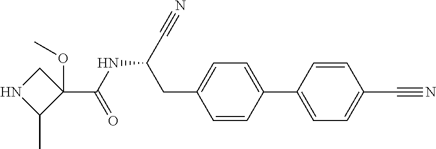 [SMILES: COC1(C(=O)N[C@H](C#N)Cc2ccc(-c3ccc(C#N)cc3)cc2)CNC1C] | Example 31 | LCMS (method AcHSSC18): [MH+] = 375.4 at 3.20 min Chiral analysis (method 2): [MH+] = 375.4 at 2.30 and 2.40 min |
| | diastereoisomers 2 and 3 of N-((S)-1- | | |
| | cyano-2-(4′-cyano-[1,1′-biphenyl]-4- | | |
| | yl)ethyl)-3-methoxy-2-methylazetidine- | | |
| | 3-carboxamide | | |
| 34 | 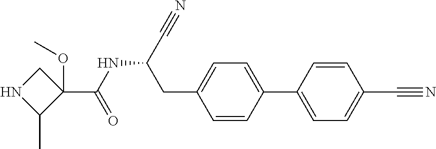 [SMILES: COC1(C(=O)N[C@H](C#N)Cc2ccc(-c3ccc(C#N)cc3)cc2)CNC1C] | Example 31 | LCMS (method AcHSSC18): [MH+] = 375.4 at 3.22 min Chiral analysis (method 2): [MH+] = 375.4 at 3.36 min |
| | diastereoisomer 4 of N-((S)-1-cyano-2- | | |
| | (4′-cyano-[1,1′-biphenyl]-4-yl)ethyl)-3- | | |
| | methoxy-2-methylazetidine-3- | | |
| | carboxamide | | |

### Pharmacological Activity of the Compounds of the Invention

### In Vitro Assays

### Activity on Human Recombinant DPP1 Isolated Enzyme Assay

Human DPP1 enzyme activity was determined by measuring release of the fluorophore aminomethyl coumarin (AMC) following enzymatic cleavage of the dipeptide substrate H-Gly-Arg-AMC. Assays were performed in black 384 well plates in 25 mM piperazine buffer, pH 5.0, containing 50 mM NaCl, 0.01% (v/v) Triton X100, 5 mM DTT with 0.35 nM human DPP1 enzyme, 300 μM H-Gly-Arg-AMC substrate (˜Km concentration) and test compounds at 0.51-10000 nM concentration range. Human DPP1 was pre-incubated with test compounds for 30 minutes at 25° C. prior to substrate incubation for a further 40 minutes at 25° C. Enzyme activity was determined by measuring fluorescence of the AMC product at excitation and emission wavelengths of 380 nm and 460 nm using a BMG LABTECH PHERA star plate reader. Test compound potencies are reported in Table 15 as pIC50 values generated from 4-parameter sigmoidal curve fitting of the measured concentration-inhibition responses.

TABLE 15

| Example N. | h DPP1 pIC₅₀ |
|---|---|
| 3, 4, 9, 10, 20, 21, 23, 26, 30, 45, 46, 51 | + |
| 1, 2, 5, 9, 29, 31, 35, 42, 52 | ++ |
| 6, 7, 8, 11, 12, 13, 14, 17, 18, 22, 38, | +++ |
| 39, 40, 43, 44 | |
| 15, 16, 24, 36, 37, 41, 47, 48, 49, 50 | ++++ |

wherein the compounds are classified in term of potency with respect to their inhibitory activity on h DPP1 enzyme according to the following classification criterion:
+: h DPP1 pIC₅₀ comprised between Sand 6
++: h DPP1 pIC₅₀ comprised between 6 and 7
+++: h DPP1 pIC₅₀ comprised between 7 and 8
++++: h DPP1 pIC₅₀ higher than 8.

As suggested in the state of the art for cyclic inhibitors of DPP1 β-amino acids, it was noted that an improvement in enzymatic and cellular potencies occurred as the ring size increased (see e.g. K Doyle, J Med. Chem. 2016, 59, 9457-9472). As representative of the compound of the invention, Example 9, where a four-member ring derivative of the invention is considered, is now compared with Compounds 14, 15, 16 reported in K Doyle, J. Med. Chemistry, 2016, 59, 9457-9472, Table 3, page 9461.

While in the case of compounds 14, 15, 16 in the human recombinant DPP1 isolated enzyme assay the values pIC50 increase from less than 5 for compounds 14 and 15 to pIC50 5.9 for compound 16, surprisingly for Example 9 of the present invention pIC50 resulted between 5 and 6, therefore higher than what is reported in the state of the art for compounds 14 and 15.

As it can be appreciated, all the compounds of Table 15 show an inhibitory activity on DPP1 enzyme. In fact, it can be recognized that the symbol + indicate a sufficient or good level of activity, which can be even increased up to ++++, thus confirming the high activity on h DPP1 enzyme of the compounds of the invention.

1: A compound of formula (I) 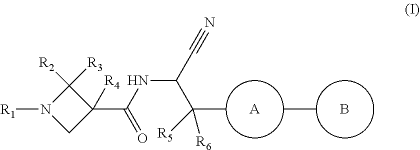
wherein:
R₁ is H or selected from the group consisting of —(C₁-C₆)alkyl, —(C₁-C₆)haloalkyl, —(C₁-C₆)hydroxyalkyl, —(C₁-C₆)alkoxy, —(C₃-C₆)cycloalkyl, aryl, heterocycloalkyl and heteroaryl, wherein said —(C₁-C₆)alkyl is optionally substituted by one or more groups selected from the group consisting of —NR₉SO₂R₁₀, —SO₂NR₉R₁₀, —NR₉—C(O)R₁₀, —C(O)NR₉R₁₀, —NR₉R₁₀, aryl, —(C₃-C₆)cycloalkyl, heterocycloalkyl and heteroaryl;
R₂ and R₃ are independently H or selected from the group consisting of —(C₁-C₆)alkyl, halogen and —(C₁-C₆)haloalkyl;
R₄ is H or selected from the group consisting of halogen, —OR₇, —SR₈, —SO₂R₈, —(C₁-C₆)alkyl, —(C₁-C₆)haloalkyl, —(C₁-C₆)aminoalkyl, —(C₁-C₆)hydroxyalkyl, —(C₁-C₆)alkoxy, —(C₁-C₆)alkyl-S—(C₁-C₄)alkyl, heterocycloalkyl, aryl, heteroaryl, and —(C₃-C₆)cycloalkyl, wherein any of such —(C₁-C₆)alkyl, —(C₁-C₆)haloalkyl, —(C₁-C₆)alkoxy, and —(C₁-C₆)alkyl-S—(C₁-C₄)alkyl are optionally substituted by one or more groups selected from the group consisting of halogen, —NR₉SO₂R₁₀, —SO₂NR₉R₁₀, —C(O)NR₉R₁₀, —NR₉R₁₀, —NR₉—C(O)R₁₀, aryl, —(C₃-C₆)cycloalkyl, heterocycloalkyl and heteroaryl;
R₅ and R₆ are independently H, —(C₁-C₆)alkyl, —(C₁-C₆)haloalkyl or fused together in a —(C₃-C₆)cycloalkyl, wherein said —(C₃-C₆)cycloalkyl is optionally substituted by one or more groups selected from the group consisting of halogen and —(C₁-C₆)alkyl;
R₇ and R₈ are independently H or selected from the group consisting of —(C₁-C₆)alkyl, —(C₁-C₆)haloalkyl, heterocycloalkyl, —(C₃-C₆)cycloalkyl, aryl, and heteroaryl, wherein said —(C₁-C₆)alkyl is optionally substituted by one or more groups selected from the group consisting of aryl, —(C₃-C₆)cycloalkyl, heterocycloalkyl, —NR₉SO₂R₁₀, —SO₂NR₉R₁₀, —C(O)NR₉R₁₀, —NR₉—C(O)R₁₀, and —NR₉R₁₀, and wherein any of such aryl, heteroaryl, —(C₃-C₆)cycloalkyl, and heterocycloalkyl are optionally substituted by one or more halogen, —(C₁-C₆)alkyl, —(C₁-C₆)haloalkyl, and/or —(C₁-C₆)hydroxyalkyl;
R₉ or R₁₀ are independently H or selected from the group consisting of —(C₁-C₆)haloalkyl, aryl, and —(C₁-C₆)alkyl or fused together in a heterocycloalkyl or in —(C₃-C₆)cycloalkyl, wherein any of such heterocycloalkyl and —(C₃-C₆)cycloalkyl are optionally substituted by one or more —(C₁-C₆)alkyl;
A is monocyclic ring selected from the group consisting of aryl, heterocycloalkyl, and —(C₃-C₆)cycloalkyl, wherein any of such aryl, heterocycloalkyl, and —(C₃-C₆)cycloalkyl are optionally substituted by one or more substituents selected from the group consisting of halogen, oxo, —OR₇, —NR₉SO₂R₁₀, —SO₂NR₉R₁₀, —C(O)NR₉R₁₀, —NR₉—C(O)R₁₀—NR₉R₁₀, —SR₈, —(C₁-C₆)alkyl, —(C₁-C₆)haloalkyl, —(C₁-C₆)hydroxyalkyl, and —(C₁-C₆)amminoalkyl;
B is a ring selected from the group consisting of aryl and heteroaryl, wherein each of said aryl or heteroaryl are optionally fused to a second saturated or unsaturated ring optionally containing one or more heteroatoms selected from the group consisting of N, S and O to form a bicyclic, tricyclic or a spiro tricyclic ring system, said B being optionally substituted with one or more substituents selected from the group consisting of halogen, —OR₇, —SR₈, oxo, cyano, —(C₁-C₆)haloalkyl, —(C₁-C₆)alkyl, —(C₁-C₆)amminoalkyl, aryl, heteroaryl, heterocycloalkyl, —(C₃-C₆)cycloalkyl, —NR₉R₁₀, —C(O)NR₉R₁₀, —NR₉—C(O)R₁₀, —SO₂NR₉R₁₀, —NR₉SO₂R₁₀, —(C₁-C₆)alkyl-C(O)NR₉R₁₀, —(C₁-C₆)alkyl-NR₉—C(O)R₁₀, —(C₁-C₆)alkyl-SO₂NR₉R₁₀, —(C₁-C₆)alkyl-NR₉SO₂R₁₀, —NH—C(O)—OR₇ and —O—C(O)—NR₉R₁₀,
or a pharmaceutically acceptable salt or deuterated form thereof.

2: The compound of formula (I), salt, or deuterated form thereof according to claim 1, wherein
R₁ is H or selected from the group consisting of —(C₁-C₆)alkyl, heterocycloalkyl and heteroaryl, wherein said —(C₁-C₆)alkyl is optionally substituted by one or more groups selected from the group consisting of aryl, —(C₃-C₆)cycloalkyl, —C(O)NR₉R₁₀, heterocycloalkyl and heteroaryl; and
R₂ and R₃ are independently H or —(C₁-C₆)alkyl.

3: The compound of formula (I), salt, or deuterated form thereof according to claim 1, wherein
R₄ is H or selected from the group consisting of halogen, —OR₇, —SR₈, —SO₂R₈, —(C₁-C₆)alkyl, —(C₁-C₆)haloalkyl, —(C₁-C₆)hydroxyalkyl, and —(C₁-C₆)alkoxy, wherein any of such —(C₁-C₆)alkyl are optionally substituted by one or more groups selected from the group consisting of aryl, —(C₁-C₆)cycloalkyl and heterocycloalkyl; and
R₅ and R₆ are H.

4: The compound of formula (I), salt, or deuterated form thereof according to claim 1, wherein
A is aryl or pyridine, wherein such aryl or pyridine is optionally substituted by one or more substituents selected from the group consisting of halogen, —(C₁-C₆)alkyl, —(C₁-C₆)haloalkyl, —(C₁-C₆)hydroxyalkyl and —(C₁-C₆)amminoalkyl.

5: The compound of formula (I), salt, or deuterated form thereof according to claim 1, wherein B is an aryl or heteroaryl ring, wherein said heteroaryl is optionally fused to a second saturated or unsaturated ring optionally containing one or more heteroatoms selected from the group consisting of N and O to form a tricyclic or spiro tricyclic ring system, said aryl or heteroaryl being optionally substituted with one or more substituents selected from the group consisting of oxo, cyano, and —(C₁-C₆)alkyl.

6: The compound of formula (I), salt, or deuterated form thereof according to claim 1, wherein
B is an aryl or heteroaryl ring, said aryl being optionally substituted with one or more substituents selected from the group consisting of oxo, cyano, and —(C₁-C₆)alkyl, and said heteroaryl selected from the group consisting of (II) (III) and (IV).

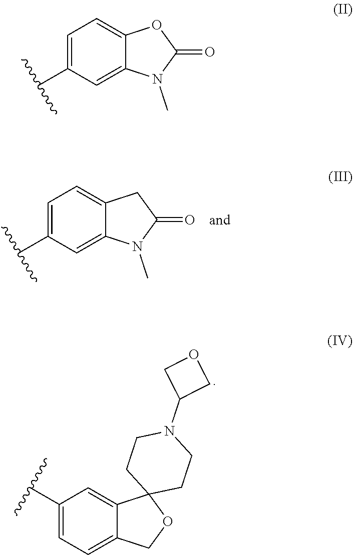 [SMILES: *c1ccc2oc(=O)n(C)c2c1 ; *c1ccc2c(c1)N(C)C(=O)C2 ; *c1ccc2c(c1)C1(CCN(C3COC3)CC1)OC2]

7: The compound of formula (I), salt, or deuterated form thereof according to claim 1, wherein the compound of formula (I) is selected from the group consisting of:
(S)—N-(1-cyano-2-(4′-cyano-[1,1′-biphenyl]-4-yl)ethyl)-3-hydroxyazetidine-3-carboxamide;
(S)—N-(1-cyano-2-(4′-cyano-[1,1′-biphenyl]-4-yl)ethyl)-3-fluoroazetidine-3-carboxamide;
N—((S)-1-cyano-2-(4′-cyano-[1,1′-biphenyl]-4-yl)ethyl)-2-methylazetidine-3-carboxamide;
N—((S)-1-cyano-2-(4′-cyano-[1,1′-biphenyl]-4-yl)ethyl)-2,2-dimethylazetidine-3-carboxamide;
(S)—N-(1-cyano-2-(4′-cyano-[1,1′-biphenyl]-4-yl)ethyl)-3-ethylazetidine-3-carboxamide;
(S)—N-(1-cyano-2-(4′-cyano-[1,1′-biphenyl]-4-yl)ethyl)-3-methoxyazetidine-3-carboxamide;
(S)—N-(1-cyano-2-(4′-cyano-[1,1′-biphenyl]-4-yl)ethyl)-3-(difluoromethyl)azetidine-3-carboxamide;
(S)—N-(1-cyano-2-(4′-cyano-[1,1′-biphenyl]-4-yl)ethyl)-3-(fluoromethyl)azetidine-3-carboxamide;
(S)—N-(1-cyano-2-(4′-cyano-[1,1′-biphenyl]-4-yl)ethyl)azetidine-3-carboxamide;
(S)—N-(1-cyano-2-(4′-cyano-[1,1′-biphenyl]-4-yl)ethyl)-3-(methoxymethyl)azetidine-3-carboxamide;
(S)—N-(1-cyano-2-(4′-cyano-[1,1′-biphenyl]-4-yl)ethyl)-3-ethoxyazetidine-3-carboxamide;
(S)—N-(1-cyano-2-(4′-cyano-3-fluoro-[1,1′-biphenyl]-4-yl)ethyl)-3-methoxyazetidine-3-carboxamide;
(S)—N-(1-cyano-2-(4′-cyano-3-fluoro-[1,1′-biphenyl]-4-yl)ethyl)-3-(difluoromethyl)azetidine-3-carboxamide;
(S)—N-(1-cyano-2-(4-(3-methyl-2-oxo-2,3-dihydrobenzo[d]oxazol-5-yl)phenyl)ethyl)-3-(difluoromethyl)azetidine-3-carboxamide;
(S)—N-(1-cyano-2-(2-fluoro-4-(1′-(oxetan-3-yl)-3H-spiro[isobenzofuran-1,4′-piperidin]-6-yl)phenyl)ethyl)-3-(difluoromethyl)azetidine-3-carboxamide;
(S)—N-(1-cyano-2-(2-fluoro-4-(1-methyl-2-oxoindolin-6-yl)phenyl)ethyl)-3-(difluoromethyl)azetidine-3-carboxamide;
(S)—N-(1-cyano-2-(4′-cyano-[1,1′-biphenyl]-4-yl)ethyl)-3-(trifluoromethyl)azetidine-3-carboxamide;
(S)—N-(1-cyano-2-(4′-cyano-[1,1′-biphenyl]-4-yl)ethyl)-3-(methylthio)azetidine-3-carboxamide;
(S)—N-(1-cyano-2-(4′-cyano-[1,1′-biphenyl]-4-yl)ethyl)-3-(methylsulfonyl)azetidine-3-carboxamide;
(S)-3-benzyl-N-(1-cyano-2-(4′-cyano-[1,1′-biphenyl]-4-yl)ethyl)azetidine-3-carboxamide;
(S)—N-(1-cyano-2-(4′-cyano-[1,1′-biphenyl]-4-yl)ethyl)-3-phenoxyazetidine-3-carboxamide;
(S)—N-(1-cyano-2-(4-(1-methyl-2-oxoindolin-6-yl)phenyl)ethyl)-3-methoxyazetidine-3-carboxamide;
(S)—N-(1-cyano-2-(4′-cyano-[1,1′-biphenyl]-4-yl)ethyl)-1-(oxetan-3-yl)azetidine-3-carboxamide;
(S)—N-(1-cyano-2-(4′-cyano-[1,1′-biphenyl]-4-yl)ethyl)-3-methoxy-1-methylazetidine-3-carboxamide;
(S)—N-(1-cyano-2-(4′-cyano-[1,1′-biphenyl]-4-yl)ethyl)-1-(furan-2-ylmethyl)azetidine-3-carboxamide;
(S)—N-(1-cyano-2-(4′-cyano-[1,1′-biphenyl]-4-yl)ethyl)-3-isopropoxyazetidine-3-carboxamide;
(S)—N-(1-cyano-2-(4′-cyano-[1,1′-biphenyl]-4-yl)ethyl)-1-(oxazol-5-ylmethyl)azetidine-3-carboxamide;
(S)—N-(1-cyano-2-(4′-cyano-[1,1′-biphenyl]-4-yl)ethyl)-1-(2-(methylamino)-2-oxoethyl)azetidine-3-carboxamide;
(S)—N-(1-cyano-2-(4′-cyano-[1,1′-biphenyl]-4-yl)ethyl)-3-methoxy-1-(oxetan-3-yl)azetidine-3-carboxamide;
(S)—N-(1-cyano-2-(4′-cyano-[1,1′-biphenyl]-4-yl)ethyl)-1-isopropyl-3-methoxyazetidine-3-carboxamide;
N—((S)-1-cyano-2-(4′-cyano-[1,1′-biphenyl]-4-yl)ethyl)-3-methoxy-2-methylazetidine-3-carboxamide;
diastereoisomer 1 of N—((S)-1-cyano-2-(4′-cyano-[1,1′-biphenyl]-4-yl)ethyl)-3-methoxy-2-methylazetidine-3-carboxamide;
mixture of diastereoisomer 2 and 3 of N—((S)-1-cyano-2-(4′-cyano-[1,1′-biphenyl]-4-yl)ethyl)-3-methoxy-2-methylazetidine-3-carboxamide;
diastereoisomer 4 of N—((S)-1-cyano-2-(4′-cyano-[1,1′-biphenyl]-4-yl)ethyl)-3-methoxy-2-methylazetidine-3-carboxamide;
(S)—N-(1-cyano-2-(4′-cyano-[1,1′-biphenyl]-4-yl)ethyl)-3-(difluoromethyl)-1-methylazetidine-3-carboxamide;
(S)—N-(1-cyano-2-(2-fluoro-4-(3-methyl-2-oxo-2,3-dihydrobenzo[d]oxazol-5-yl)phenyl)ethyl)-3-methoxyazetidine-3-carboxamide;
(S)—N-(1-cyano-2-(2-fluoro-4-(3-methyl-2-oxo-2,3-dihydrobenzo[d]oxazol-5-yl)phenyl)ethyl)-3-methoxy-1-methylazetidine-3-carboxamide;
(S)—N-(1-cyano-2-(4′-cyano-[1,1′-biphenyl]-4-yl)ethyl)-3-hydroxy-1-methylazetidine-3-carboxamide;
(S)—N-(1-cyano-2-(2-fluoro-4-(1′-(oxetan-3-yl)-3H-spiro[isobenzofuran-1,4′-piperidin]-6-yl)phenyl)ethyl)-3-(difluoromethyl)-1-methylazetidine-3-carboxamide;
(S)—N-(1-cyano-2-(4′-cyano-3-fluoro-[1,1′-biphenyl]-4-yl)ethyl)-3-(difluoromethyl)-1-methylazetidine-3-carboxamide;
(S)—N-(1-cyano-2-(4′-cyano-3-fluoro-[1,1′-biphenyl]-4-yl)ethyl)-3-methoxy-1-methylazetidine-3-carboxamide;
N-(1-cyano-2-(5-(4-cyanophenyl)-3-fluoropyridin-2-yl)ethyl)-3-methoxyazetidine-3-carboxamide;
N-(1-cyano-2-(5-(4-cyanophenyl)-3-fluoropyridin-2-yl)ethyl)-3-methoxy-1-methylazetidine-3-carboxamide;
Enantiomer 1 of N-(1-cyano-2-(5-(4-cyanophenyl)-3-fluoropyridin-2-yl)ethyl)-3-methoxy-1-methylazetidine-3-carboxamide;
Enantiomer 2 of N-(1-cyano-2-(5-(4-cyanophenyl)-3-fluoropyridin-2-yl)ethyl)-3-methoxy-1-methylazetidine-3-carboxamide;
(S)—N-(1-cyano-2-(4′-cyano-[1,1′-biphenyl]-4-yl)ethyl)-3-methoxy-1-propylazetidine-3-carboxamide;
(S)—N-(1-cyano-2-(2-fluoro-4-(1′-(oxetan-3-yl)-3H-spiro[isobenzofuran-1,4′-piperidin]-6-yl)phenyl)ethyl)-3-methoxy-1-methylazetidine-3-carboxamide;
(S)—N-(1-cyano-2-(2-fluoro-4-(1-methyl-2-oxoindolin-6-yl)phenyl)ethyl)-3-(fluoromethyl)azetidine-3-carboxamide;
(S)—N-(1-cyano-2-(2-fluoro-4-(1-methyl-2-oxoindolin-6-yl)phenyl)ethyl)-3-methoxyazetidine-3-carboxamide;
(S)—N-(1-cyano-2-(2-fluoro-4-(1-methyl-2-oxoindolin-6-yl)phenyl)ethyl)-3-methoxy-1-methylazetidine-3-carboxamide;
(S)—N-(1-cyano-2-(4′-cyano-[1,1′-biphenyl]-4-yl)ethyl)-1-isobutyl-3-methoxyazetidine-3-carboxamide;
(S)—N-(1-cyano-2-(4′-cyano-[1,1′-biphenyl]-4-yl)ethyl)-1-ethyl-3-methoxyazetidine-3-carboxamide; and
(S)—N-(1-cyano-2-(2-fluoro-4-(1′-(oxetan-3-yl)-3H-spiro[isobenzofuran-1,4′-piperidin]-6-yl)phenyl)ethyl)-3-methoxyazetidine-3-carboxamide.

8: A pharmaceutical composition comprising the compound of formula (I), salt, or deuterated form thereof according to claim 1, in admixture with one or more pharmaceutically acceptable carriers or excipients.

9. (canceled)

10: A method for treating a disease, disorder, or condition associated with dysregulation of DDP1, comprising administering the compound of formula (I), salt, or deuterated form thereof according to claim 1 to a subject in need thereof.

11: A method of treating an inflammatory or obstructive respiratory disease, comprising administering the compound of formula (I), salt, or deuterated form thereof according to claim 1 to a subject in need thereof.

12: The method of claim 11, wherein the inflammatory or obstructive respiratory disease is selected from the group consisting of: asthma, chronic obstructive pulmonary disease (COPD), Non-cystic fibrosis bronchiectasis (NCFBE), chronic bronchitis, pneumonia, acute respiratory distress syndrome (ARDS), Acute lung injury (ALI), lung fibrosis, idiopathic pulmonary fibrosis, pulmonary emphysema, smoking-induced emphysema and cystic fibrosis.

13: The method of claim 11, wherein the inflammatory or obstructive respiratory disease comprises non-cystic fibrosis bronchiectasis (NCFBE).

14: The for method of claim 10, wherein the administering comprises oral administration.

15: The method of claim 11, wherein the administering comprises oral administration.
